(19) United States
(12) Patent Application Publication
(10) Pub. No.: US 2025/0197393 A1
(43) Pub. Date: Jun. 19, 2025

(54) INTEGRIN INHIBITOR AND USES THEREOF

(71) Applicant: Pliant Therapeutics, Inc., South San Francisco, CA (US)

(72) Inventors: Meiqi LI, Shanghai (CN); Xiaoyu MA, Wuhan (CN)

(21) Appl. No.: 18/981,111

(22) Filed: Dec. 13, 2024

(30) Foreign Application Priority Data
Dec. 14, 2023 (WO) PCT/CN2023/138817

(51) Int. Cl. C07D 471/04; A61K 31/517

(52) U.S. Cl. CPC C07D 471/04; A61K 31/517

(57) ABSTRACT

Provided herein are crystalline forms of integrin inhibitors, compositions thereof, and methods of their uses. Crystalline forms of tartrate cocrystals of the inhibitors are also described, along with methods of preparing the crystalline forms. X-ray powder diffraction data, thermogravimetric analysis, and differential scanning calorimetry data are provided for the crystalline forms. The integrin inhibitors are useful for treatment of, inter alia, fibrotic diseases.

### CROSS-REFERENCE TO RELATED APPLICATIONS

This patent application claims priority to and the benefit of International Patent Application No. PCT/CN2023/138817 filed Dec. 14, 2023, the entire contents of which are hereby incorporated by reference.

### FIELD

Provided herein are integrin inhibitors, compositions thereof, and methods of their uses.

### BACKGROUND

Fibrosis, a pathologic feature of many diseases, is caused by a dysfunction in the body's natural ability to repair damaged tissues. If left untreated, fibrosis can result in scarring of vital organs causing irreparable damage and eventual organ failure.

Patients with nonalcoholic fatty liver disease (NAFLD) may progress from simple steatosis to nonalcoholic steatohepatitis (NASH) and then fibrosis. While liver fibrosis is reversible in its initial stages, progressive liver fibrosis can lead to cirrhosis.

Fibrosis in the kidney, characterized by glomerulosclerosis and tubulointerstitial fibrosis, is the final common manifestation of a wide variety of chronic kidney diseases (CKD). Irrespective of the initial causes, progressive CKD often results in widespread tissue scarring that leads to destruction of kidney parenchyma and end-stage renal failure, a devastating condition that requires dialysis or kidney replacement.

Scleroderma encompasses a spectrum of complex and variable conditions primarily characterized by fibrosis, vascular alterations, and autoimmunity. The scleroderma spectrum of disorders share the common feature of fibrosis, resulting in hardening or thickening of the skin. For some patients, this hardening occurs only in limited areas, but for others, it can spread to other major organs.

Following myocardial infarction, cardiac structural remodeling is associated with an inflammatory reaction, resulting in scar formation at the site of the infarction. This scar formation is a result of fibrotic tissue deposition which may lead to reduced cardiac function and disruption of electrical activity within the heart.

Crohn's Disease is a chronic disease of unknown etiology tending to progress even in the setting of medical or surgical treatment. Intestinal fibrosis is among the most common complications of Crohn's disease, resulting in stricture formation in the small intestine and colon.

Idiopathic pulmonary fibrosis (IPF) is a chronic, progressive, fibrosing disease of unknown etiology, occurring in adults and limited to the lungs. In IPF, the lung tissue becomes thickened, stiff, and scarred. As lung fibrosis progresses, it becomes more difficult for the lungs to transfer oxygen into the bloodstream and the organs do not receive the oxygen needed to function properly. IPF currently affects approximately 200,000 people in the U.S., resulting in 40,000 deaths per year. Patients diagnosed with IPF experience progressive breathlessness and eventually, complete respiratory failure.

Primary biliary cholangitis (PBC), also known as primary biliary cirrhosis, is a chronic disease of the liver that causes damage and fibrosis in the liver. It results from a slow, progressive destruction of the small bile ducts of the liver, causing bile and other toxins to build up in the liver, a condition called cholestasis. Over time, this leads to scarring and fibrosis in both the liver and biliary tract. Primary sclerosing cholangitis (PSC) involves bile duct inflammation, and fibrosis that obliterates the bile ducts. The resulting impediment to the flow of bile to the intestines can lead to cirrhosis of the liver and subsequent complications such as liver failure and liver cancer.

Nonspecific interstitial pneumonia (NSIP) is a rare disorder that affects the tissue that surrounds and separates the tiny air sacs of the lungs. These air sacs, called the alveoli, are where the exchange of oxygen and carbon dioxide takes place between the lungs and the bloodstream. Interstitial pneumonia is a disease in which the mesh-like walls of the alveoli become inflamed. The pleura (a thin covering that protects and cushions the lungs and the individual lobes of the lungs) might become inflamed as well. There are two primary forms of NSIP-cellular and fibrotic. The cellular form is defined mainly by inflammation of the cells of the interstitium. The fibrotic form is defined by thickening and scarring of lung tissue. This scarring is known as fibrosis. When the lung tissue thickens or becomes scarred, it does not function as effectively. Breathing becomes less efficient, and there are lower levels of oxygen in the blood. (Kim et al., Proc. Am. Thorac. Soc. (2006) 3:285-292; Lynch, D., Radiology (2001) 221:583-584; Kinder et al., Am. J. Respir. Crit. Care Med. (2007) 176:691-697).

The αᵥβ₆ integrin is expressed in epithelial cells, and binds to the latency-associated peptide of transforming growth factor-β1 (TGFβ1) and mediates TGFβ1 activation. Its expression level is significantly increased after injury to lung and cholangiocytes, and plays a critical in vivo role in tissue fibrosis. Increased levels are also associated with increased mortality in IPF and NSIP patients. Expression of αᵥβ₆ is also elevated in liver and bile duct of PSC patients.

There remains a great need for agents that exploit new mechanisms of action and may have better outcomes in terms of relief of symptoms, safety, and patient mortality, both short-term and long-term. US Pat. Pub. No. 2019/0276449, now U.S. Pat. No. 10,793,564, the contents of each of which are incorporated herein by reference in their entireties, discloses (S)-4-((2-methoxyethyl)(4-(5,6,7,8-tetrahydro-1,8-naphthyridin-2-yl)butyl)amino)-2-(quinazolin-4-ylamino)butanoic acid, which is an αᵥβ₆ integrin inhibitor and provides a potential treatment of fibrosis.

To move a drug candidate such as (S)-4-((2-methoxyethyl)(4-(5,6,7,8-tetrahydro-1,8-naphthyridin-2-yl)butyl)amino)-2-(quinazolin-4-ylamino)butanoic acid to a viable pharmaceutical product, it can be important to understand whether the drug candidate has crystalline forms, as well as the relative stability and interconversions of these forms under conditions likely to be encountered upon large-scale production, transportation, storage and pre-usage preparation. The ability to control and produce a stable crystalline form with a robust manufacturing process can be key for regulatory approval and marketing, batch-to-batch reproducibility, and quality control for the amount of active pharmaceutical ingredient in the drug product, which can be important for patient outcomes. Large scale production processes for high purity (S)-4-((2-methoxyethyl)(4-(5,6,7,8-tetrahydro-1,8-naphthyridin-2-yl)butyl)amino)-2-(quinazolin-4-ylamino)butanoic acid can be improved by use of particular crystalline forms. Accordingly, it is desirable to identify new crystalline forms of (S)-4-((2-methoxyethyl)(4-(5,6,7,8-tetrahydro-1,8-naphthyridin-2-yl)butyl)amino)-2-(quinazolin-4-ylamino)butanoic acid with different chemical and physical stabilities, and compositions and uses of the same.

### BRIEF SUMMARY

In one aspect, provided herein is a crystalline form of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, as detailed herein.

In one aspect, provided herein is a crystalline form of (S)-4-((2-methoxyethyl)(4-(5,6,7,8-tetrahydro-1,8-naphthyridin-2-yl)butyl)amino)-2-(quinazolin-4-ylamino)butanoic acid with tartaric acid, or a solvate thereof, as detailed herein. In one aspect, the tartaric acid is L-tartaric acid.

In one aspect, provided herein is a cocrystal form of (S)-4-((2-methoxyethyl)(4-(5,6,7,8-tetrahydro-1,8-naphthyridin-2-yl)butyl)amino)-2-(quinazolin-4-ylamino)butanoic acid with tartaric acid, or a solvate thereof, as detailed herein. In one aspect, the molar ratio of (S)-4-((2-methoxyethyl)(4-(5,6,7,8-tetrahydro-1,8-naphthyridin-2-yl)butyl)amino)-2-(quinazolin-4-ylamino)butanoic acid to tartaric acid is 1:2 or about 1:2. In one aspect, the approximate molar ratio of (S)-4-((2-methoxyethyl)(4-(5,6,7,8-tetrahydro-1,8-naphthyridin-2-yl)butyl)amino)-2-(quinazolin-4-ylamino)butanoic acid to tartaric acid is 1:1.8 to 1:2.2, 1:1.9 to 1:2.1, 1:1.95 to 1:2.05, or 1:1.98 to 1:2.02. In one aspect, the tartaric acid is L-tartaric acid.

In another aspect, provided herein is a method of preparing a crystalline form of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, as detailed herein.

In one aspect, provided herein is a method of preparing a crystalline form of (S)-4-((2-methoxyethyl)(4-(5,6,7,8-tetrahydro-1,8-naphthyridin-2-yl)butyl)amino)-2-(quinazolin-4-ylamino)butanoic acid, or a hydrate thereof, comprising preparing a mixture of (S)-4-((2-methoxyethyl)(4-(5,6,7,8-tetrahydro-1,8-naphthyridin-2-yl)butyl)amino)-2-(quinazolin-4-ylamino)butanoic acid and tartaric acid in a solvent, as detailed herein. In one aspect, the tartaric acid is L-tartaric acid.

In one aspect, provided herein is a method of preparing a cocrystal form of (S)-4-((2-methoxyethyl)(4-(5,6,7,8-tetrahydro-1,8-naphthyridin-2-yl)butyl)amino)-2-(quinazolin-4-ylamino)butanoic acid with tartaric acid, or a solvate thereof, as detailed herein. In one aspect, the molar ratio of (S)-4-((2-methoxyethyl)(4-(5,6,7,8-tetrahydro-1,8-naphthyridin-2-yl)butyl)amino)-2-(quinazolin-4-ylamino)butanoic acid to tartaric acid is 1:2 or about 1:2. In one aspect, the approximate molar ratio of (S)-4-((2-methoxyethyl)(4-(5,6,7,8-tetrahydro-1,8-naphthyridin-2-yl)butyl)amino)-2-(quinazolin-4-ylamino)butanoic acid to tartaric acid is 1:1.8 to 1:2.2, 1:1.9 to 1:2.1, 1:1.95 to 1:2.05, or 1:1.98 to 1:2.02. In one aspect, the tartaric acid is L-tartaric acid.

In another aspect, provided herein is a composition comprising a crystalline form of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, and a pharmaceutically acceptable carrier or excipient, as detailed herein.

In one aspect, provided herein is a composition comprising a cocrystal form of (S)-4-((2-methoxyethyl)(4-(5,6,7,8-tetrahydro-1,8-naphthyridin-2-yl)butyl)amino)-2-(quinazolin-4-ylamino)butanoic acid with tartaric acid, or a solvate thereof, and a pharmaceutically acceptable carrier or excipient, as detailed herein. In one aspect, the molar ratio of (S)-4-((2-methoxyethyl)(4-(5,6,7,8-tetrahydro-1,8-naphthyridin-2-yl)butyl)amino)-2-(quinazolin-4-ylamino)butanoic acid to tartaric acid is 1:2 or about 1:2. In one aspect, the approximate molar ratio of (S)-4-((2-methoxyethyl)(4-(5,6,7,8-tetrahydro-1,8-naphthyridin-2-yl)butyl)amino)-2-(quinazolin-4-ylamino)butanoic acid to tartaric acid is 1:1.8 to 1:2.2, 1:1.9 to 1:2.1, 1:1.95 to 1:2.05, or 1:1.98 to 1:2.02. In one aspect, the tartaric acid is L-tartaric acid.

In another aspect, provided herein is a kit comprising a crystalline form of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, and instructions for use in, e.g., treating a fibrotic disease, as detailed herein.

In one aspect, provided herein is a kit comprising a cocrystal form of (S)-4-((2-methoxyethyl)(4-(5,6,7,8-tetrahydro-1,8-naphthyridin-2-yl)butyl)amino)-2-(quinazolin-4-ylamino)butanoic acid with tartaric acid, or a solvate thereof, and instructions for use in, e.g., treating a fibrotic disease, as detailed herein. In one aspect, the molar ratio of (S)-4-((2-methoxyethyl)(4-(5,6,7,8-tetrahydro-1,8-naphthyridin-2-yl)butyl)amino)-2-(quinazolin-4-ylamino)butanoic acid to tartaric acid is 1:2 or about 1:2. In one aspect, the approximate molar ratio of (S)-4-((2-methoxyethyl)(4-(5,6,7,8-tetrahydro-1,8-naphthyridin-2-yl)butyl)amino)-2-(quinazolin-4-ylamino)butanoic acid to tartaric acid is 1:1.8 to 1:2.2, 1:1.9 to 1:2.1, 1:1.95 to 1:2.05, or 1:1.98 to 1:2.02. In one aspect, the tartaric acid is L-tartaric acid.

In one aspect, provided herein is a crystalline form of a salt of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, as detailed herein. In one aspect, provided herein is a composition comprising a crystalline form of a salt of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, and a pharmaceutically acceptable carrier or excipient.

In another aspect, provided herein is a method of preparing a crystalline form of a salt of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, as detailed herein. In another aspect, provided herein is a method of preparing a composition comprising a crystalline form of a salt of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, and a pharmaceutically acceptable carrier or excipient.

In another aspect, provided herein is a composition comprising a crystalline form of a salt of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, as detailed herein. In another aspect, provided herein is a composition comprising a crystalline form of a salt of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, and a pharmaceutically acceptable carrier or excipient.

In another aspect, provided herein is a kit comprising a crystalline form of a salt of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, as detailed herein. In another aspect, provided herein is a kit comprising a composition comprising a crystalline form of a salt of (S)-4-((2-methoxyethyl)(4-(5,6,7,8-tetrahydro-1,8-naphthyridin-2-yl)butyl)amino)-2-(quinazolin-4-ylamino)butanoic acid or a solvate thereof, and a pharmaceutically acceptable carrier or excipient.

In another aspect, provided herein is a method of treating a fibrotic disease in an individual (such as a human) in need thereof comprising administering to the individual a crystalline form or a composition disclosed herein. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC).

In another aspect, provided herein is a method of treating a fibrotic disease in an individual (such as a human) in need thereof comprising administering to the individual an effective amount of a crystalline form or a composition disclosed herein. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC).

In another aspect, provided herein is a method of treating a fibrotic disease in an individual (such as a human) in need thereof comprising administering to the individual a cocrystal form or a composition comprising a cocrystal form disclosed herein. In some embodiments, the disease is fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, biliary fibrosis, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, or Crohn's Disease. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC).

In another aspect, provided herein is a method of treating a fibrotic disease in an individual (such as a human) in need thereof comprising administering to the individual an effective amount of a cocrystal form or a composition comprising a cocrystal form disclosed herein. In some embodiments, the disease is fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, biliary fibrosis, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, or Crohn's Disease. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC).

In another aspect, provided is a method of delaying the onset and/or development of a fibrotic disease in an individual (such as a human) who is at risk for developing a fibrotic disease comprising administering to the individual an effective amount of a crystalline form or a composition disclosed herein. In some embodiments, the disease is fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, biliary fibrosis, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, or Crohn's Disease. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or PBC. In some embodiments, the individual at risk of developing a fibrotic disease has or is suspected of having NAFLD, NASH, CKD, scleroderma, Crohn's Disease, NSIP, PSC, PBC, or is an individual who has had or is suspected of having had a myocardial infarction.

In another aspect, provided is a method of delaying the onset and/or development of a fibrotic disease in an individual (such as a human) who is at risk for developing a fibrotic disease comprising administering to the individual an effective amount of a cocrystal form or a composition comprising a cocrystal form disclosed herein. In some embodiments, the disease is fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, biliary fibrosis, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, or Crohn's Disease. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or PBC. In some embodiments, the individual at risk of developing a fibrotic disease has or is suspected of having NAFLD, NASH, CKD, scleroderma, Crohn's Disease, NSIP, PSC, PBC, or is an individual who has had or is suspected of having had a myocardial infarction.

In some embodiments, provided is a method of treating or delaying the onset and/or development of a disease, where the disease is selected from the group consisting of a fibrotic disease, pulmonary fibrosis, idiopathic pulmonary fibrosis (IPF), progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), biliary fibrosis, primary biliary cholangitis (PBC, also known as primary biliary cirrhosis), biliary atresia, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease (CKD), myocardial infarction, and Crohn's Disease.

In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC).

In some embodiments, the fibrotic disease is a pulmonary fibrosis, e.g., idiopathic pulmonary fibrosis (IPF). In some embodiments, the pulmonary fibrosis is, e.g., interstitial lung disease, radiation-induced pulmonary fibrosis, or systemic sclerosis associated interstitial lung disease. In some embodiments, the pulmonary fibrosis is IPF, progressive pulmonary fibrosis, rheumatoid arthritis-associated interstitial lung disease, interstitial lung disease, radiation-induced pulmonary fibrosis, or systemic sclerosis associated interstitial lung disease.

In some embodiments, the fibrotic disease is a primary sclerosing cholangitis, or biliary fibrosis. In some embodiments, the fibrotic disease is primary biliary cholangitis (also known as primary biliary cirrhosis) or biliary atresia.

In some embodiments, the fibrotic disease is fibrotic nonspecific interstitial pneumonia (NSIP).

In some embodiments, the fibrotic disease is a liver fibrosis, e.g., infectious liver fibrosis (i.e., liver fibrosis induced from pathogens, such as HCV, HBV or parasites such as schistosomiasis), NASH, alcoholic steatosis induced liver fibrosis, alcoholic liver disease induced fibrosis, and cirrhosis. In some embodiments, the liver fibrosis is nonalcoholic fatty liver disease (NAFLD). In some embodiments, the liver fibrosis is NASH.

In some embodiments, the fibrotic disease is biliary tract fibrosis.

In some embodiments, the fibrotic disease is renal fibrosis, e.g., diabetic nephrosclerosis, hypertensive nephrosclerosis, focal segmental glomerulosclerosis (“FSGS”), and acute kidney injury from contrast induced nephropathy. In several embodiments, the fibrotic disease is diabetic nephropathy, diabetic kidney disease, or chronic kidney disease.

In some embodiments, the fibrotic disease is characterized by one or more of glomerulonephritis, end-stage kidney disease, hearing loss, changes to the lens of the eye, hematuria, or proteinuria. In some embodiments, the fibrotic disease is Alport syndrome.

In some embodiments, the fibrotic disease is systemic and local sclerosis or scleroderma, keloids and hypertrophic scars, or post-surgical adhesions. In some embodiments, the fibrotic disease is scleroderma or systemic sclerosis.

In some embodiments, the fibrotic disease is atherosclerosis or restenosis.

In some embodiments, the fibrotic disease is a gastrointestinal fibrosis, e.g., Crohn's disease.

In some embodiments, the fibrotic disease is cardiac fibrosis, e.g., post myocardial infarction induced fibrosis and inherited cardiomyopathy.

Also provided is a crystalline form or a composition disclosed herein for use in the treatment of a fibrotic disease. Also provided is use of a crystalline form or a composition disclosed herein for use in the treatment of a fibrotic disease. Also provided is use of a crystalline form or a composition disclosed herein in the manufacture of a medicament for the treatment of a fibrotic disease.

Also provided is a cocrystal form or a composition comprising a cocrystal form disclosed herein for use in the treatment of a fibrotic disease. Also provided is use of a cocrystal form or a composition comprising a cocrystal form disclosed herein in the treatment of a fibrotic disease. Also provided is use of a cocrystal form or a composition comprising a cocrystal form disclosed herein in the manufacture of a medicament for the treatment of a fibrotic disease.

### BRIEF DESCRIPTION OF THE DRAWINGS

FIG. 1 shows the XRPD pattern of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A.

FIG. 2 shows a differential scanning calorimetry (DSC) graph of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A.

FIG. 3 shows a thermogravimetric analysis (TGA) graph of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A.

FIG. 4 shows the XRPD pattern of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B.

FIG. 5 shows a differential scanning calorimetry (DSC) graph of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B.

FIG. 6 shows a thermogravimetric analysis (TGA) graph of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B.

FIG. 7 shows the XRPD pattern of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C.

FIG. 8 shows a differential scanning calorimetry (DSC) graph of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C.

FIG. 9 shows a thermogravimetric analysis (TGA) graph of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C.

FIG. 10 shows a Fourier transform infrared (FTIR) spectrum of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A.

FIG. 11 shows dynamic vapor sorption (DVS) results for (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A.

### DETAILED DESCRIPTION

### Definitions

As used herein, unless clearly indicated otherwise, use of the terms “a”, “an” and the like refers to one or more.

As used herein, reference to “about” a value or parameter herein includes (and describes) embodiments that are directed to that value or parameter per se. For example, description referring to “about X” includes description of “X”. As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 20%, within 15%, within 10%, within 5%, within 4%, within 3%, within 2%, within 1%, or within 0.5% of the specified dose, amount, or weight percent.

As used herein, the term “crystalline form” refers to a crystalline solid form of a chemical compound, including, but not limited to, a single-component or multiple-component crystal form, e.g., a polymorph of a compound; or a solvate, a hydrate, a clathrate, a cocrystal, a salt of a compound, or a polymorph thereof. The term “crystal forms” and related terms herein refers to the various crystalline modifications of a given substance, including, but not limited to, polymorphs, solvates, hydrates, cocrystals and other molecular complexes, as well as salts, solvates of salts, hydrates of salts, other molecular complexes of salts, and polymorphs thereof. Crystal forms of a substance can be obtained by a number of methods, as known in the art. Such methods include, but are not limited to, melt recrystallization, melt cooling, solvent recrystallization, slurrying, recrystallization in confined spaces such as, e.g., in nanopores or capillaries, recrystallization on surfaces or templates such as, e.g., on polymers, recrystallization in the presence of additives, such as, e.g., anti-solvents, cocrystal counter-molecules, desolvation, dehydration, rapid evaporation, rapid cooling, slow cooling, vapor diffusion, sublimation, grinding and solvent-drop grinding.

As used herein, “solvate” encompasses solvates, partial solvates, and channel solvates. As such, a solvate need not contain an exact stoichiometric ratio of compound:solvent but may include ratios of compound:solvent permitted by experimental variance. The term “solvate” is further intended to include aqueous and non-aqueous solvated forms (e.g., hydrates, ethanolates, etc.). Thus, it is understood that a solvate encompasses stoichiometric solvates, channel solvates and partial solvates. It is also understood that a hydrate encompasses stoichiometric hydrates, channel hydrates and partial hydrates.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing the effect of another medication, delaying the progression of the disease, increasing the quality of life, and/or prolonging survival. Also encompassed by “treatment” is a reduction of pathological consequence of fibrosis. The methods of the invention contemplate any one or more of these aspects of treatment.

As used herein, the term “effective amount” intends such amount of a compound of the invention which should be effective in a given therapeutic form. As is understood in the art, an effective amount may be in one or more doses, e.g., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

As used herein, a “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, the term “controlled release” refers to a drug-containing formulation or fraction thereof in which release of the drug is not immediate, e.g., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration, herein incorporated by reference in its entirety.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

Unless otherwise stated, “substantially pure” intends a composition that contains no more than about 10% impurity, such as a composition comprising less than about 9%, about 7%, about 5%, about 3%, about 1%, or about 0.5% impurity.

It is understood that aspects and embodiments described herein as “comprising” include “consisting of” and “consisting essentially of” embodiments.

As used herein, the term “substantially as shown in” when referring, for example, to an XRPD pattern, a DSC graph, a TGA graph, or a GVS graph, includes a pattern or graph that is not necessarily identical to those depicted herein, but that falls within the limits of experimental error or deviations when considered by one of ordinary skill in the art.

### Crystalline Forms

In one aspect, provided herein is a crystalline form of a salt of (S)-4-((2-methoxyethyl)(4-(5,6,7,8-tetrahydro-1,8-naphthyridin-2-yl)butyl)amino)-2-(quinazolin-4-ylamino)butanoic acid, a compound having the structure shown below, with L-tartaric acid, or a solvate thereof:

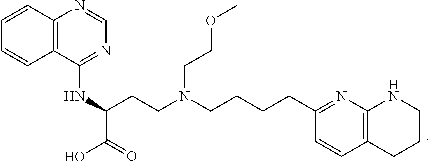 [SMILES: COCCN(CCCCc1ccc2c(n1)NCCC2)CC[C@H](Nc1ncnc2ccccc12)C(=O)O]

In some embodiments, the crystalline form of the salt of (S)-4-((2-methoxyethyl)(4-(5,6,7,8-tetrahydro-1,8-naphthyridin-2-yl)butyl)amino)-2-(quinazolin-4-ylamino)butanoic acid with L-tartaric acid is a hydrate. In some embodiments, the crystalline form the salt is (S)-4-((2-methoxyethyl)(4-(5,6,7,8-tetrahydro-1,8-naphthyridin-2-yl)butyl)amino)-2-(quinazolin-4-ylamino)butanoic acid with L-tartaric acid.

In one aspect, provided herein is a crystalline form, of (S)-4-((2-methoxyethyl)(4-(5,6,7,8-tetrahydro-1,8-naphthyridin-2-yl)butyl)amino)-2-(quinazolin-4-ylamino)butanoic acid, a compound having the structure shown below, with L-tartaric acid, or a solvate thereof:

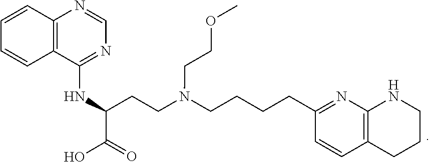 [SMILES: COCCN(CCCCc1ccc2c(n1)NCCC2)CC[C@H](Nc1ncnc2ccccc12)C(=O)O]

In some embodiments, the crystalline form of (S)-4-((2-methoxyethyl)(4-(5,6,7,8-tetrahydro-1,8-naphthyridin-2-yl)butyl)amino)-2-(quinazolin-4-ylamino)butanoic acid with L-tartaric acid is a hydrate. In some embodiments, the crystalline form is (S)-4-((2-methoxyethyl)(4-(5,6,7,8-tetrahydro-1,8-naphthyridin-2-yl)butyl)amino)-2-(quinazolin-4-ylamino)butanoic acid with L-tartaric acid.

As used herein, a reference to the “parent compound” means the free form of (S)-4-((2-methoxyethyl)(4-(5,6,7,8-tetrahydro-1,8-naphthyridin-2-yl)butyl)amino)-2-(quinazolin-4-ylamino)butanoic acid, and not a cocrystal, salt, hydrate, or solvate thereof. Likewise, reference to “(S)-4-((2-methoxyethyl)(4-(5,6,7,8-tetrahydro-1,8-naphthyridin-2-yl)butyl)amino)-2-(quinazolin-4-ylamino)butanoic acid” alone or its structural formula alone will be a reference to the parent compound, unless otherwise noted or made clear in the context in which the reference is used. US Pat. Pub. No. 2019/0276449, now U.S. Pat. No. 10,793,564, the contents of each of which are incorporated herein by reference in their entireties, discloses preparation of (S)-4-((2-methoxyethyl)(4-(5,6,7,8-tetrahydro-1,8-naphthyridin-2-yl)butyl)amino)-2-(quinazolin-4-ylamino)butanoic acid.

As used herein, amounts of (S)-4-((2-methoxyethyl)(4-(5,6,7,8-tetrahydro-1,8-naphthyridin-2-yl)butyl)amino)-2-(quinazolin-4-ylamino)butanoic acid tartaric acid crystalline form are on a parent basis, unless indicated otherwise. For example, the molecular weight of anhydrous(S)-4-((2-methoxyethyl)(4-(5,6,7,8-tetrahydro-1,8-naphthyridin-2-yl)butyl)amino)-2-(quinazolin-4-ylamino)butanoic acid cocrystal with two equivalents of tartaric acid is approximately 792.8, while the molecular weight of the parent(S)-4-((2-methoxyethyl)(4-(5,6,7,8-tetrahydro-1,8-naphthyridin-2-yl)butyl)amino)-2-(quinazolin-4-ylamino)butanoic acid is approximately 492.62. Thus, 161 mg of anhydrous(S)-4-((2-methoxyethyl)(4-(5,6,7,8-tetrahydro-1,8-naphthyridin-2-yl)butyl)amino)-2-(quinazolin-4-ylamino)butanoic acid cocrystal with two equivalents of tartaric acid is equivalent to 100 mg of (S)-4-((2-methoxyethyl)(4-(5,6,7,8-tetrahydro-1,8-naphthyridin-2-yl)butyl)amino)-2-(quinazolin-4-ylamino)butanoic acid. Thus, as would be understood by the ordinarily skilled artisan, unless indicated otherwise, the dosage amounts of crystalline form as used herein refer to the dosage amount of (S)-4-((2-methoxyethyl)(4-(5,6,7,8-tetrahydro-1,8-naphthyridin-2-yl)butyl)amino)-2-(quinazolin-4-ylamino)butanoic acid. A corresponding amount of tartaric acid cocrystal or other form of (S)-4-((2-methoxyethyl)(4-(5,6,7,8-tetrahydro-1,8-naphthyridin-2-yl)butyl)amino)-2-(quinazolin-4-ylamino)butanoic acid may be readily determined.

L-tartaric acid (also known as (2R,3R)-2,3-dihydroxysuccinic acid, (2R,3R)-2,3-dihydroxybutanedioic acid, or L-(+)-tartaric acid) has the following structure:

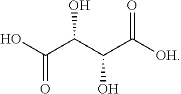

In one aspect, provided herein is a cocrystal of (S)-4-((2-methoxyethyl)(4-(5,6,7,8-tetrahydro-1,8-naphthyridin-2-yl)butyl)amino)-2-(quinazolin-4-ylamino)butanoic acid with L-tartaric acid, or solvate thereof.

In one aspect, provided herein is a cocrystal of (S)-4-((2-methoxyethyl)(4-(5,6,7,8-tetrahydro-1,8-naphthyridin-2-yl)butyl)amino)-2-(quinazolin-4-ylamino)butanoic acid, with L-tartaric acid, or solvate thereof, in a 1:2 molar ratio or an approximate 1:2 molar ratio of (S)-4-((2-methoxyethyl)(4-(5,6,7,8-tetrahydro-1,8-naphthyridin-2-yl)butyl)amino)-2-(quinazolin-4-ylamino)butanoic acid to L-tartaric acid (e.g., the cocrystal has one molecule, or about one molecule, of (S)-4-((2-methoxyethyl)(4-(5,6,7,8-tetrahydro-1,8-naphthyridin-2-yl)butyl)amino)-2-(quinazolin-4-ylamino)butanoic acid, per two molecules, or about two molecules, of L-tartaric acid). In one aspect, the approximate molar ratio of (S)-4-((2-methoxyethyl)(4-(5,6,7,8-tetrahydro-1,8-naphthyridin-2-yl)butyl)amino)-2-(quinazolin-4-ylamino)butanoic acid to tartaric acid is 1:1.8 to 1:2.2, 1:1.9 to 1:2.1, 1:1.95 to 1:2.05, or 1:1.98 to 1:2.02.

The crystalline forms disclosed herein may provide the advantages of bioavailability and stability and may be suitable for use as an active agent in a pharmaceutical composition. Variations in the crystal structure of a pharmaceutical drug substance may affect the dissolution rate (which may affect bioavailability, etc.), manufacturability (e.g., ease of handling, ease of purification, ability to consistently prepare doses of known strength, etc.) and stability (e.g., thermal stability, shelf life (including resistance to oxidative degradation), etc.) of a pharmaceutical drug product. Such variations may affect the methods of preparation or formulation of pharmaceutical compositions in different dosage or delivery forms, such as solid oral dosage forms including tablets and capsules. Compared to other forms such as non-crystalline or amorphous forms, crystalline forms may provide desired or suitable hygroscopicity, particle size control, dissolution rate, solubility, purity, physical and chemical stability, manufacturability, yield, reproducibility, and/or process control. Thus, the crystalline forms disclosed herein may provide advantages over the parent form, salts thereof, or other crystalline forms of any of the foregoing, of improving the manufacturing process of an active agent or the stability or storability of a drug product form of the active agent or having suitable bioavailability and/or stability as an active agent.

### Pattern A Crystalline Form

In some embodiments, provided herein is a crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid designated as Pattern A. Methods of making Pattern A are exemplified herein; see Example 1, Example 2, and Example 3.

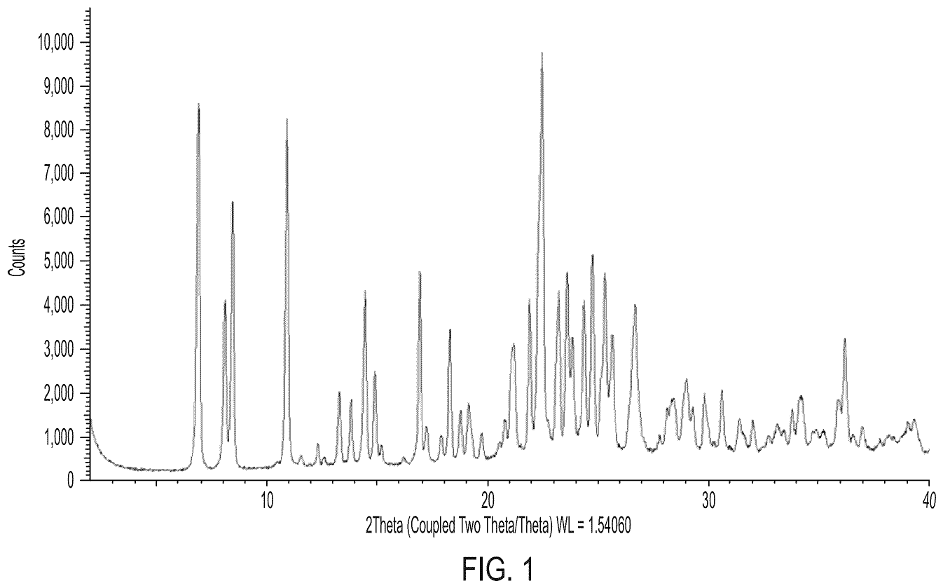

In some embodiments, Pattern A has an XRPD pattern substantially as shown in FIG. 1. In some embodiments, Pattern A has an XRPD pattern comprising one or more (e.g., at least one, at least two, at least three, at least four, at least five, at least six, at least seven, at least eight, at least nine, or at least ten) of the peaks at angles 2-theta corresponding to the peaks of greatest relative intensity in the XRPD pattern substantially as shown in FIG. 1. In some embodiments, Pattern A has an XRPD pattern comprising one or more (e.g., at least one, at least two, at least three, at least four, at least five, at least six, at least seven, at least eight, at least nine, or at least ten) of the peaks at angles 2-theta of 21.9±0.2 degrees, 23.23±0.2 degrees, 23.65±0.2 degrees, 16.93±0.2 degrees, 25.35±0.2 degrees, 24.78±0.2 degrees, 8.47±0.2 degrees, 6.94±0.2 degrees, 10.93±0.2 degrees, or 22.46±0.2 degrees. In some embodiments, Pattern A has an XRPD pattern comprising one or more (e.g., at least one, at least two, at least three, at least four, at least five, at least six, at least seven, at least eight, at least nine, or at least ten) of the peaks at angles 2-theta of about 21.9 degrees, about 23.23 degrees, about 23.65 degrees, about 16.93 degrees, about 25.35 degrees, about 24.78 degrees, about 8.47 degrees, about 6.94 degrees, about 10.93 degrees, or about 22.46 degrees. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Pattern A, can independently vary by ±0.6 degrees, +0.4 degrees, +0.2 degrees, or +0.1 degrees 2-theta. In some embodiments, each peak assignment listed herein, including for Pattern A, can independently vary by ±0.2 degrees 2-theta.

In some embodiments, Pattern A has an XRPD pattern comprising one or more (e.g., at least one, at least two, at least three, at least four, at least five, at least six, at least seven, at least eight, at least nine, or at least ten) of the peaks at angles 2-theta corresponding to the peaks of greatest relative intensities in the XRPD pattern substantially as shown in Table 1. In some embodiments, Pattern A has an XRPD pattern comprising one or more (e.g., at least one, at least nine, or at least ten) of the peaks at angles 2-theta of 21.9±0.2 degrees, 23.23±0.2 degrees, 23.65±0.2 degrees, 16.93±0.2 degrees, 25.35±0.2 degrees, 24.78±0.2 degrees, 8.47±0.2 degrees, 6.94±0.2 degrees, 10.93±0.2 degrees, or 22.46±0.2 degrees. In some embodiments, Pattern A has an XRPD pattern comprising one or more (e.g., at least one, at least two, at least three, at least four, at least five, at least six, at least seven, at least eight, at least nine, or at least ten) of the peaks at angles 2-theta of about 21.9 degrees, about 23.23 degrees, about 23.65 degrees, about 16.93 degrees, about 25.35 degrees, about 24.78 degrees, about 8.47 degrees, about 6.94 degrees, about 10.93 degrees, or about 22.46 degrees. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Pattern A, can independently vary by +0.6 degrees, +0.4 degrees, +0.2 degrees, or +0.1 degrees 2-theta. In some embodiments, each peak assignment listed herein, including for Pattern A, can independently vary by +0.2 degrees 2-theta.

TABLE 1

| | | Angle | d | | Rel. | |
|---|---|---|---|---|---|---|
| | | 2-Theta | Value | | Intensity | |
| Index | Name | (degrees) | (Å) | | (%)* | Intensity |
| 4 | Peak#4 | 10.544 | 8.383 | | 1.00 | 400.97 |
| 8 | Peak#8 | 12.596 | 7.02169 | | 1.50 | 498.328 |
| 14 | Peak#14 | 16.203 | 5.466 | | 1.70 | 529.728 |
| 6 | Peak#6 | 11.578 | 7.6369 | | 2.50 | 564.202 |
| 13 | Peak#13 | 15.207 | 5.82169 | | 4.60 | 796.49 |
| 7 | Peak#7 | 12.323 | 7.1768 | | 5.30 | 842.467 |
| 22 | Peak#22 | 20.561 | 4.31623 | | 3.40 | 865.651 |
| 57 | Peak#57 | 38.407 | 2.34186 | | 2.50 | 929.327 |
| 55 | Peak#55 | 37.774 | 2.37962 | | 2.60 | 950.093 |
| 35 | Peak#35 | 27.827 | 3.20346 | | 3.30 | 965.983 |
| 44 | Peak#44 | 32.775 | 2.73028 | | 3.00 | 986.681 |
| 17 | Peak#17 | 17.919 | 4.94621 | | 5.90 | 991.474 |
| 53 | Peak#53 | 36.549 | 2.45658 | | 2.90 | 1005.77 |
| 46 | Peak#46 | 33.445 | 2.67711 | | 3.10 | 1025.42 |
| 50 | Peak#50 | 35.211 | 2.54678 | | 2.90 | 1028.53 |
| 56 | Peak#56 | 38.182 | 2.35514 | | 3.60 | 1035.09 |
| 21 | Peak#21 | 19.736 | 4.49471 | | 6.00 | 1059.78 |
| 49 | Peak#49 | 34.882 | 2.57006 | | 3.40 | 1069.4 |
| 23 | Peak#23 | 20.771 | 4.27311 | | 5.90 | 1108.2 |
| 54 | Peak#54 | 36.978 | 2.42905 | | 4.20 | 1115.78 |
| 45 | Peak#45 | 33.144 | 2.70074 | | 4.80 | 1169.15 |
| 42 | Peak#42 | 31.428 | 2.8442 | | 5.20 | 1176.35 |
| 58 | Peak#58 | 39.041 | 2.30531 | | 5.60 | 1190 |
| 16 | Peak#16 | 17.249 | 5.13665 | | 8.40 | 1202.13 |
| 59 | Peak#59 | 39.312 | 2.29002 | | 5.80 | 1202.16 |
| 19 | Peak#19 | 18.779 | 4.72151 | | 8.10 | 1228.79 |
| 43 | Peak#43 | 32.024 | 2.79258 | | 5.90 | 1242 |
| 36 | Peak#36 | 28.158 | 3.16656 | | 7.4 | 1359.81 |
| 10 | Peak#10 | 13.827 | 6.39933 | | 10.60 | 1362.52 |
| 20 | Peak#20 | 19.143 | 4.63262 | | 9.50 | 1367.1 |
| 47 | Peak#47 | 33.814 | 2.64869 | | 6.80 | 1376.12 |
| 51 | Peak#51 | 35.95 | 2.49606 | | 6.90 | 1388.74 |
| 39 | Peak#39 | 29.319° | 3.04378 | Å | 7.70% | 1416.02 |
| 37 | Peak#37 | 28.372° | 3.14314 | Å | 8.20% | 1439.66 |
| 48 | Peak#48 | 34.194° | 2.62013 | Å | 8.10% | 1496.89 |
| 9 | Peak#9 | 13.303° | 6.65026 | Å | 12.30% | 1510.25 |
| 40 | Peak#40 | 29.845° | 2.9913 | Å | 9.90% | 1619.78 |

| | | | | | |
|---|---|---|---|---|---|
| 41 | Peak#41 | 30.631 | 2.91631 | 11.20 | 1739.9 |
| 12 | Peak#12 | 14.91 | 5.93704 | 15.60 | 1816.68 |
| 38 | Peak#38 | 29.036 | 3.07275 | 12.20 | 1822.81 |
| 24 | Peak#24 | 21.15 | 4.19735 | 18.20 | 2271.11 |
| 18 | Peak#18 | 18.295 | 4.84549 | 22.10 | 2510.33 |
| 29 | Peak#29 | 23.874 | 3.72425 | 19.80 | 2537.06 |
| 33 | Peak#33 | 25.672 | 3.46727 | 20.40 | 2591.38 |
| 52 | Peak#52 | 36.19 | 2.48006 | 20.80 | 2665.79 |
| 2 | Peak#2 | 8.118 | 10.88231 | 27.70 | 2836.31 |
| 34 | Peak#34 | 26.712 | 3.33458 | 24.90 | 2977.09 |
| 30 | Peak#30 | 24.412 | 3.64329 | 25.10 | 3036.26 |
| 11 | Peak#11 | 14.451 | 6.12439 | 29.30 | 3086.21 |
| 25 | Peak#25 | 21.9 | 4.05526 | 27.60 | 3187.7 |
| 27 | Peak#27 | 23.225 | 3.82671 | 27.70 | 3256.89 |
| 28 | Peak#28 | 23.652 | 3.75864 | 30.20 | 3492.94 |
| 15 | Peak#15 | 16.93 | 5.23292 | 33.50 | 3503.8 |
| 32 | Peak#32 | 25.351 | 3.51045 | 30.50 | 3528.61 |
| 31 | Peak#31 | 24.779 | 3.59016 | 34.00 | 3856.36 |
| 3 | Peak#3 | 8.472 | 10.42877 | 44.20 | 4356.87 |
| 1 | Peak#1 | 6.936 | 12.73427 | 58.50 | 5670.55 |
| 5 | Peak#5 | 10.934 | 8.0852 | 57.90 | 5674.97 |
| 26 | Peak#26 | 22.457 | 3.95589 | 100.00 | 9907.23 |

*Relative intensity is calculated based on net peak intensity. Net peak intensity is calculated after background removal from peak intensity.

In some embodiments, Pattern A is characterized by an XRPD pattern comprising:
a. peaks at angles 2-theta of 8.11±0.2 degrees and 10.93±0.2 degrees;
b. peaks at angles 2-theta of 8.11±0.2 degrees, 10.93±0.2 degrees, and 14.91±0.2 degrees; or
c. peaks at angles 2-theta of 8.11±0.2 degrees, 10.93±0.2 degrees, 14.91±0.2 degrees, and 22.46±0.2 degrees. In some embodiments, Pattern A is characterized by an XRPD pattern comprising peaks at angles 2-theta of 8.11±0.2 degrees and 10.93±0.2 degrees. In some embodiments, Pattern A is characterized by an XRPD pattern comprising peaks at angles 2-theta of 8.11±0.2 degrees, 10.93±0.2 degrees, and 14.91±0.2 degrees. In some embodiments, Pattern A is characterized by an XRPD pattern comprising peaks at angles 2-theta of 8.11±0.2 degrees, 10.93±0.2 degrees, 14.91±0.2 degrees, and 22.46±0.2 degrees.

In some embodiments, Pattern A is characterized by an XRPD pattern comprising:
a. peaks at angles 2-theta of about 8.11 degrees and about 10.93 degrees;
b. peaks at angles 2-theta of about 8.11 degrees, about 10.93 degrees, and about 14.91 degrees; or
c. peaks at angles 2-theta of about 8.11 degrees, about 10.93 degrees, about 14.91 degrees, and about 22.46 degrees. In some embodiments, Pattern A is characterized by an XRPD pattern comprising peaks at angles 2-theta of about 8.11 degrees and about 10.93 degrees. In some embodiments, Pattern A is characterized by an XRPD pattern comprising peaks at angles 2-theta of about 8.11 degrees, about 10.93 degrees, and about 14.91 degrees. In some embodiments, Pattern A is characterized by an XRPD pattern comprising peaks at angles 2-theta of about 8.11 degrees, about 10.93 degrees, about 14.91 degrees, and about 22.46 degrees.

In some embodiments, Pattern A is characterized by an XRPD pattern comprising:
a. peaks at angles 2-theta of 6.94±0.2 degrees and 22.46±0.2 degrees;
b. peaks at angles 2-theta of 6.94±0.2 degrees, 10.93±0.2 degrees, and 22.46±0.2 degrees; or
c. peaks at angles 2-theta of 6.94±0.2 degrees, 8.47±0.2 degrees, 10.93±0.2 degrees, 22.46±0.2 degrees, and 24.78±0.2 degrees. In some embodiments, Pattern A is characterized by an XRPD pattern comprising peaks at angles 2-theta of 6.94±0.2 degrees and 22.46±0.2 degrees. In some embodiments, Pattern A is characterized by an XRPD pattern comprising peaks at angles 2-theta of 6.94±0.2 degrees, 10.93±0.2 degrees, and 22.46±0.2 degrees. In some embodiments, Pattern A is characterized by an XRPD pattern comprising peaks at angles 2-theta of 6.94±0.2 degrees, 8.47±0.2 degrees, 10.93±0.2 degrees, 22.46±0.2 degrees, and 24.78±0.2 degrees.

In some embodiments, Pattern A is characterized by an XRPD pattern comprising:
a. peaks at angles 2-theta of about 6.94 degrees and about 22.46 degrees;
b. peaks at angles 2-theta of about 6.94 degrees, about 10.93 degrees, and about 22.46 degrees; or
c. peaks at angles 2-theta of about 6.94 degrees, about 8.47 degrees, about 10.93 degrees, about 22.46 degrees, and about 24.78 degrees. In some embodiments, Pattern A is characterized by an XRPD pattern comprising peaks at angles 2-theta of about 6.94 degrees and about 22.46 degrees. In some embodiments, Pattern A is characterized by an XRPD pattern comprising peaks at angles 2-theta of about 6.94 degrees, about 10.93 degrees, and about 22.46 degrees. In some embodiments, Pattern A is characterized by an XRPD pattern comprising peaks at angles 2-theta of about 6.94 degrees, about 8.47 degrees, about 10.93 degrees, about 22.46 degrees, and about 24.78 degrees.

In some embodiments, Pattern A is characterized by an XRPD pattern comprising:
a. peaks at angles 2-theta of 6.94±0.2 degrees and 10.93±0.2 degrees;
b. peaks at angles 2-theta of 6.94±0.2 degrees, 8.47±0.2 degrees, and 10.93±0.2 degrees; or
c. peaks at angles 2-theta of 6.94±0.2 degrees, 8.47±0.2 degrees, 10.93±0.2 degrees, 14.45±0.2 degrees, and 14.91±0.2 degrees. In some embodiments, Pattern A is characterized by an XRPD pattern comprising peaks at angles 2-theta of 6.94±0.2 degrees and 10.93±0.2 degrees. In some embodiments, Pattern A is characterized by an XRPD pattern comprising peaks at angles 2-theta of 6.94±0.2 degrees, 8.47±0.2 degrees, and 10.93±0.2 degrees. In some embodiments, Pattern A is characterized by an XRPD pattern comprising peaks at angles 2-theta of 6.94±0.2 degrees, 8.47±0.2 degrees, 10.93±0.2 degrees, 14.45±0.2 degrees, and 14.91±0.2 degrees.

In some embodiments, Pattern A is characterized by an XRPD pattern comprising:
a. peaks at angles 2-theta of about 6.94 degrees and about 10.93 degrees;
b. peaks at angles 2-theta of about 6.94 degrees, about 8.47 degrees, and about 10.93 degrees; or
c. peaks at angles 2-theta of about 6.94 degrees, about 8.47 degrees, about 10.93 degrees, about 14.45 degrees, and about 14.91 degrees. In some embodiments, Pattern A is characterized by an XRPD pattern comprising peaks at angles 2-theta of about 6.94 degrees and about 10.93 degrees. In some embodiments, Pattern A is characterized by an XRPD pattern comprising peaks at angles 2-theta of about 6.94 degrees, about 8.47 degrees, and about 10.93 degrees. In some embodiments, Pattern A is characterized by an XRPD pattern comprising peaks at angles 2-theta of about 6.94 degrees, about 8.47 degrees, about 10.93 degrees, about 14.45 degrees, and about 14.91 degrees.

In some embodiments, Pattern A is characterized by an XRPD pattern comprising peaks at angles 2-theta of 8.11±0.2 degrees, 10.93±0.2 degrees, 14.91±0.2 degrees, 22.46±0.2 degrees, and 24.41±0.2 degrees. In some embodiments, Pattern A is characterized by an XRPD pattern comprising peaks at angles 2-theta of about 8.11 degrees, about 10.93 degrees, about 14.91 degrees, about 22.46 degrees, and about 24.41 degrees.

In some embodiments, Pattern A is characterized by an XRPD pattern comprising one or more (e.g., at least one, at least two, at least three, at least four, at least five, at least six, at least seven, at least eight, or at least nine) peaks at angles 2-theta of 6.94±0.2 degrees, 8.11±0.2 degrees, 8.47±0.2 degrees, 10.93±0.2 degrees, 14.45±0.2 degrees, 14.91±0.2 degrees, 22.46±0.2 degrees, 24.41±0.2 degrees, or 24.78±0.2 degrees. In some embodiments, Pattern A is characterized by an XRPD pattern comprising one or more (e.g., at least one, at least two, at least three, at least four, at least five, at least six, at least seven, at least eight, or at least nine) peaks at angles 2-theta of About 6.94 degrees, about 8.11 degrees, about 8.47 degrees, about 10.93 degrees, about 14.45 degrees, about 14.91 degrees, about 22.46 degrees, about 24.41 degrees, or about 24.78 degrees.

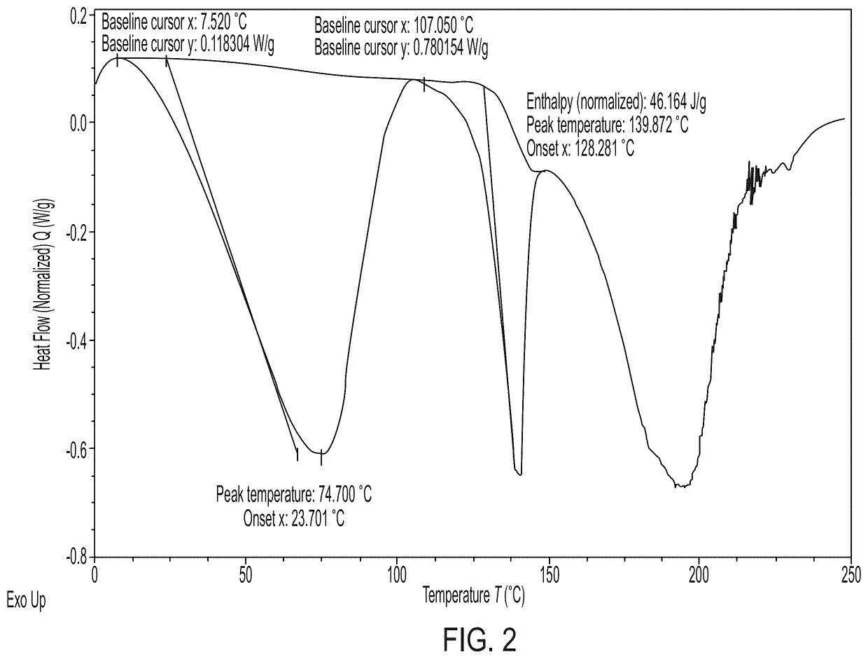

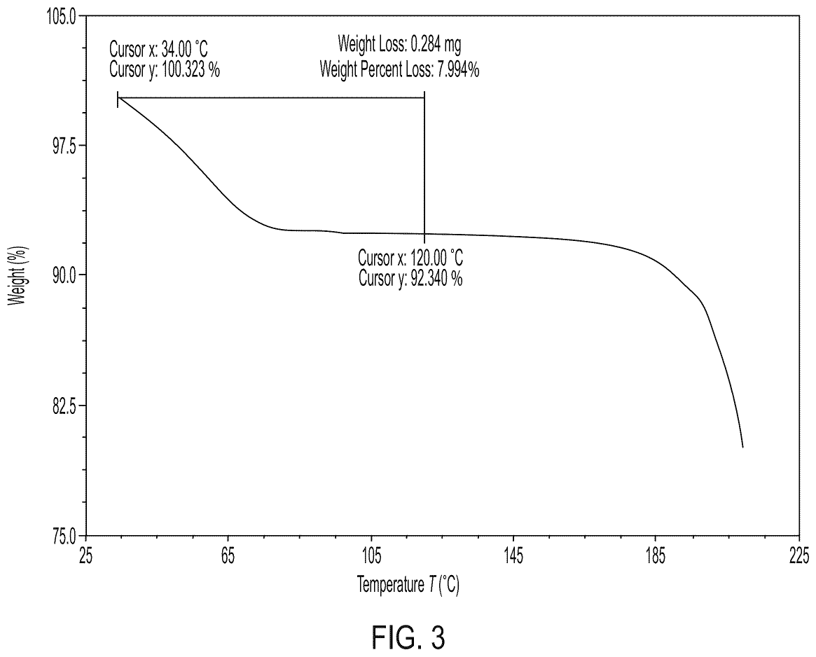

In some embodiments, Pattern A has a DSC graph substantially as shown in FIG. 2. In some embodiments, Pattern A is characterized by an endotherm peak between about 70° C. and about 80° C., and an endotherm peak between about 135° C. and about 145° C., as determined by DSC. In some embodiments, Pattern A is characterized by an endotherm peak between about 72° C. and about 77° C., and an endotherm peak between about 137° C. and about 143° C., as determined by DSC. In some embodiments, Pattern A is characterized by endotherm peaks at about 74.7° C. and about 139.9° C., as determined by DSC. In some embodiments, Pattern A has a TGA graph substantially as shown in FIG. 3. In some embodiments, Pattern A is characterized by a weight loss of about 4% to about 12% after heating from a temperature between about 30° C. and about 40° C. to a temperature between about 115° C. to about 125° C., as determined by TGA. In some embodiments, Pattern A is characterized by a weight loss of about 6% to about 10% after heating from a temperature between about 32° C. and about 36° C. to a temperature between about 118° C. to about 122° C., as determined by TGA. In some embodiments, Pattern A is characterized by a weight loss of about 8% after heating from about 34° C. to about 120° C., as determined by TGA.

In some embodiments of Pattern A, at least one, at least two, or all of the following (a)-(c) apply:
(a) Pattern A has an XRPD pattern substantially as shown in FIG. 1;
(b) Pattern A has a DSC graph substantially as shown in FIG. 2;
(c) Pattern A has a TGA graph substantially as shown in FIG. 3.

In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A is a solvate. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A is a hydrate. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A is a channel hydrate. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A contains about 4.5 equivalents (9.2% by weight) of water as measured by Karl Fischer (KF) analysis.

Pattern A may be obtained from most of the solvent systems via equilibration, slow cooling, anti-solvent addition, and vapor diffusion methods. In some embodiments, Pattern A shows a broad dehydration peak from about 8° C. as measured by DSC. In some embodiments, Pattern A melts at Tₒₙₛₑₜ of about 128.3° C. with an enthalpy of about 46 J/g. In some embodiments, Pattern A shows about 8.0% weight loss at about 120° C. as measured by TGA. In some embodiments, ¹H-NMR for Pattern A shows stoichiometry of (S)-4-((2-methoxyethyl)(4-(5,6,7,8-tetrahydro-1,8-naphthyridin-2-yl)butyl)amino)-2-(quinazolin-4-ylamino)butanoic acid free form: L-tartaric acid is 1:2.0 on a molar basis and 0.4% isopropyl alcohol (IPA) residue by weight (0.06 equiv. by molar ratio). In some embodiments, water activity experiments show that Pattern A is stable across a wide water activity (a.w.) range of a.w. ≤0.8 at 5° C., a.w. ≤0.8 at 25° C. and a.w. ≤1.0 at 50° C. In some embodiments, Pattern A shows a reversible hydration-dehydration behavior. In some embodiments, Pattern A is slightly hygroscopic with about 1.6% water uptake from 40% RH to 70% RH at 25° C. In some embodiments, Pattern A undergoes dehydration and loses about 5% water when relative humidity is below 10% RH, and the water is absorbed back after the relative humidity returns to 10% RH and above.

In some embodiments, Pattern A is characterized by one or more of 99.4% purity as measured by HPLC, a dehydration peak from about 8° C. as measured by DSC, melts at Tₒₙₛₑₜ of about 128.3° C. with an enthalpy of about 46 J/g as measured by DSC, about 8.0% weight loss at about 120° C. as measured by TGA, stoichiometry of (S)-4-((2-methoxyethyl)(4-(5,6,7,8-tetrahydro-1,8-naphthyridin-2-yl)butyl)amino)-2-(quinazolin-4-ylamino)butanoic acid free form: L-tartaric acid of 1:2.0 on a molar basis as determined by ¹H-NMR, 0.4% isopropyl alcohol (IPA) residue by weight (0.06 equiv. by molar ratio) as determined by ¹H-NMR, about 4.5 equivalents (9.2% by weight) of water as measured by Karl Fischer (KF) analysis, and aggregated irregular particles ranging from about 1 μm to 80 μm as measured by polarized light microscopy (PLM).

### Pattern B Crystalline Form

In some embodiments, provided herein is a crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid designated as Pattern B. A method of making Pattern B is shown in Example 4.

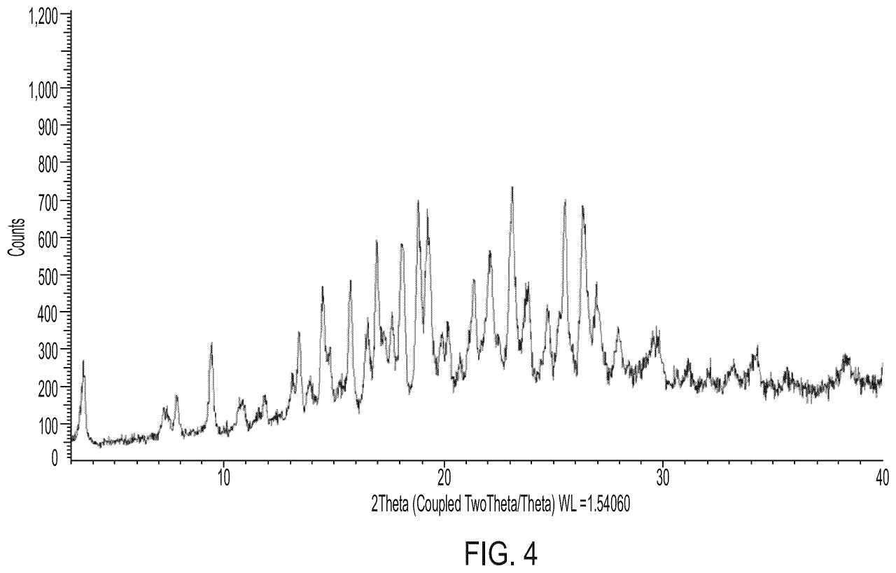

In some embodiments, Pattern B has an XRPD pattern substantially as shown in FIG. 4. In some embodiments, Pattern B has an XRPD pattern comprising one or more (e.g., at least one, at least two, at least three, at least four, at least five, at least six, at least seven, at least eight, at least nine, or at least ten) of the peaks at angles 2-theta corresponding to the peaks of greatest relative intensities in the XRPD pattern substantially as shown in FIG. 4. In some embodiments, Pattern B has an XRPD pattern comprising one or more (e.g., at least one, at least two, at least three, at least four, at least five, at least six, at least seven, at least eight, at least nine, or at least ten) of the peaks at angles 2-theta of 16.98±0.2 degrees, 18.11±0.2 degrees, 18.86±0.2 degrees, 19.28±0.2 degrees, 21.39±0.2 degrees, 22.12±0.2 degrees, 23.12±0.2 degrees, 25.53±0.2 degrees, 26.38±0.2 degrees, or 26.96±0.2 degrees. In some embodiments, Pattern B has an XRPD pattern comprising one or more (e.g., at least one, at least two, at least three, at least four, at least five, at least six, at least seven, at least eight, at least nine, or at least ten) of the peaks at angles 2-theta of about 16.98 degrees, about 18.11 degrees, about 18.86 degrees, about 19.28 degrees, about 21.39 degrees, about 22.12 degrees, about 23.12 degrees, about 25.53 degrees, about 26.38 degrees, or about 26.96 degrees. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Pattern B, can independently vary by +0.6 degrees, +0.4 degrees, +0.2 degrees, or +0.1 degrees 2-theta. In some embodiments, each peak assignment listed herein, including for Pattern B, can independently vary by ±0.2 degrees 2-theta.

In some embodiments, Pattern B has an XRPD pattern comprising one or more (e.g., at least one, at least two, at least three, at least four, at least five, at least six, at least seven, at least eight, at least nine, or at least ten) of the peaks at angles 2-theta corresponding to the peaks of greatest relative intensities in the XRPD pattern substantially as shown in Table 2. In some embodiments, Pattern B has an XRPD pattern comprising one or more (e.g., at least one, at least nine, or at least ten) of the peaks at angles 2-theta of 16.98±0.2 degrees, 18.11±0.2 degrees, 18.86±0.2 degrees, 19.28±0.2 degrees, 21.39±0.2 degrees, 22.12±0.2 degrees, 23.12±0.2 degrees, 25.53±0.2 degrees, 26.38±0.2 degrees, or 26.96±0.2 degrees. In some embodiments, Pattern B has an XRPD pattern comprising one or more (e.g., at least one, at least two, at least three, at least four, at least five, at least six, at least seven, at least eight, at least nine, or at least ten) of the peaks at angles 2-theta of about 16.98 degrees, about 18.11 degrees, about 18.86 degrees, about 19.28 degrees, about 21.39 degrees, about 22.12 degrees, about 23.12 degrees, about 25.53 degrees, about 26.38 degrees, or about 26.96 degrees. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Pattern B, can independently vary by +0.6 degrees, +0.4 degrees, +0.2 degrees, or +0.1 degrees 2-theta. In some embodiments, each peak assignment listed herein, including for Pattern B, can independently vary by +0.2 degrees 2-theta.

TABLE 2

| | | Angle | d | Rel. | |
|---|---|---|---|---|---|
| | | 2-Theta | Value | Intensity | |
| Index | Name | (dagrees) | (Å) | (%)* | Intensity |
| 2 | Peak#2 | 7.339 | 12.03551 | 13.40 | 112.613 |
| 3 | Peak#3 | 7.846 | 11.2585 | 18.00 | 134.177 |
| 5 | Peak#5 | 10.803 | 8.18284 | 14.80 | 140.533 |
| 6 | Peak#6 | 11.845 | 7.46563 | 13.10 | 151.923 |
| 1 | Peak#1 | 3.611 | 24.44747 | 34.50 | 177.523 |
| 9 | Peak#9 | 13.91 | 6.36144 | 12.10 | 181.939 |
| 7 | Peak#7 | 13.162 | 6.72125 | 19.70 | 197.652 |
| 4 | Peak#4 | 9.417 | 9.38361 | 42.00 | 226.558 |
| 35 | Peak#35 | 32.13 | 2.78356 | 9.80 | 229.063 |
| 36 | Peak#36 | 33.1 | 2.69867 | 10.60 | 232.846 |
| 39 | Peak#39 | 38.423 | 2.34093 | 9.80 | 233.452 |
| 38 | Peak#38 | 35.628 | 2.51792 | 12.50 | 238.812 |
| 33 | Peak#33 | 30.636 | 2.91583 | 9.90 | 240.802 |
| 21 | Peak#21 | 20.778 | 4.27166 | 8.70 | 246.269 |
| 37 | Peak#37 | 34.221 | 2.61816 | 14.20 | 249.541 |
| 11 | Peak#11 | 14.82 | 5.97262 | 29.20 | 252.704 |
| 34 | Peak#34 | 31.138 | 2.87 | 16.60 | 259.508 |
| 8 | Peak#8 | 13.418 | 6.59343 | 39.10 | 271.735 |
| 20 | Peak#20 | 20.204 | 4.39163 | 22.10 | 290.105 |
| 32 | Peak#32 | 29.735 | 3.00215 | 22.00 | 295.853 |
| 19 | Peak#19 | 19.919 | 4.45381 | 24.30 | 296.979 |
| 24 | Peak#24 | 22.515 | 3.94581 | 19.70 | 299.694 |
| 13 | Peak#13 | 16.539 | 5.35552 | 43.50 | 319.022 |
| 31 | Peak#31 | 27.94 | 3.19078 | 25.20 | 320.714 |
| 10 | Peak#10 | 14.524 | 6.09366 | 51.70 | 330.829 |
| 15 | Peak#15 | 17.658 | 5.01873 | 41.00 | 332.518 |
| 26 | Peak#26 | 23.798 | 3.73595 | 38.10 | 365.33 |
| 27 | Peak#27 | 24.718 | 3.59895 | 39.40 | 372.024 |
| 12 | Peak#12 | 15.763 | 5.61752 | 64.00 | 382.58 |
| 22 | Peak#22 | 21.386 | 4.1515 | 50.10 | 401.572 |
| 30 | Peak#30 | 26.955 | 3.30513 | 53.70 | 426.626 |
| 23 | Peak#23 | 22.119 | 4.0156 | 59.70 | 441.419 |
| 16 | Peak#16 | 18.11 | 4.89453 | 70.40 | 444.796 |
| 14 | Peak#14 | 16.975 | 5.21899 | 85.50 | 479.156 |
| 29 | Peak#29 | 26.383 | 3.37542 | 72.30 | 495.248 |
| 17 | Peak#17 | 18.858 | 4.70205 | 93.70 | 537.732 |
| 28 | Peak#28 | 25.525 | 3.48688 | 84.50 | 538.099 |
| 18 | Peak#18 | 19.283 | 4.5993 | 95.60 | 548.363 |
| 25 | Peak#25 | 23.122 | 3.84362 | 100.00 | 588.409 |

*Relative intensity is calculated based on net peak intensity. Net peak intensity is calculated after background removal from peak intensity.

In some embodiments, Pattern B is characterized by an XRPD pattern comprising:
a. peaks at angles 2-theta of 3.61±0.2 degrees and 7.34±0.2 degrees;
b. peaks at angles 2-theta of 3.61±0.2 degrees, 7.34±0.2 degrees, and 9.42±0.2 degrees; or
c. peaks at angles 2-theta of 3.61±0.2 degrees, 7.34±0.2 degrees, 9.42±0.2 degrees, and 15.76±0.2 degrees. In some embodiments, Pattern B is characterized by an XRPD pattern comprising peaks at angles 2-theta of 3.61±0.2 degrees and 7.34±0.2 degrees. In some embodiments, Pattern B is characterized by an XRPD pattern comprising peaks at angles 2-theta of 3.61±0.2 degrees, 7.34±0.2 degrees, and 9.42±0.2 degrees. In some embodiments, Pattern B is characterized by an XRPD pattern comprising peaks at angles 2-theta of 3.61±0.2 degrees, 7.34±0.2 degrees, 9.42±0.2 degrees, and 15.76±0.2 degrees.

In some embodiments, Pattern B is characterized by an XRPD pattern comprising:
a. peaks at angles 2-theta of about 3.61 degrees and about 7.34 degrees;
b. peaks at angles 2-theta of about 3.61 degrees, about 7.34 degrees, and about 9.42 degrees; or
c. peaks at angles 2-theta of about 3.61 degrees, about 7.34 degrees, about 9.42 degrees, and about 15.76 degrees. In some embodiments, Pattern B is characterized by an XRPD pattern comprising peaks at angles 2-theta of about 3.61 degrees and about 7.34 degrees. In some embodiments, Pattern B is characterized by an XRPD pattern comprising peaks at angles 2-theta of about 3.61 degrees, about 7.34 degrees, and about 9.42 degrees. In some embodiments, Pattern B is characterized by an XRPD pattern comprising peaks at angles 2-theta of about 3.61 degrees, about 7.34 degrees, about 9.42 degrees, and about 15.76 degrees.

In some embodiments, Pattern B is characterized by an XRPD pattern comprising:
a. peaks at angles 2-theta of 19.28±0.2 degrees and 23.12±0.2 degrees;
b. peaks at angles 2-theta of 18.86±0.2 degrees, 19.28±0.2 degrees, and 23.12±0.2 degrees; or
c. peaks at angles 2-theta of 16.98±0.2 degrees, 18.86±0.2 degrees, 19.28±0.2 degrees, 23.12±0.2 degrees, and 25.53±0.2 degrees. In some embodiments, Pattern B is characterized by an XRPD pattern comprising peaks at angles 2-theta of 19.28±0.2 degrees and 23.12±0.2 degrees. In some embodiments, Pattern B is characterized by an XRPD pattern comprising peaks at angles 2-theta of 18.86±0.2 degrees, 19.28±0.2 degrees, and 23.12±0.2 degrees. In some embodiments, Pattern B is characterized by an XRPD pattern comprising peaks at angles 2-theta of 16.98±0.2 degrees, 18.86±0.2 degrees, 19.28±0.2 degrees, 23.12±0.2 degrees, and 25.53±0.2 degrees.

In some embodiments, Pattern B is characterized by an XRPD pattern comprising:
a. peaks at angles 2-theta of about 19.28 degrees and about 23.12 degrees;
b. peaks at angles 2-theta of about 18.86 degrees, about 19.28 degrees, and about 23.12 degrees; or
c. peaks at angles 2-theta of about 16.98 degrees, about 18.86 degrees, about 19.28 degrees, about 23.12 degrees, and about 25.53 degrees. In some embodiments, Pattern B is characterized by an XRPD pattern comprising peaks at angles 2-theta of about 19.28 degrees and about 23.12 degrees. In some embodiments, Pattern B is characterized by an XRPD pattern comprising peaks at angles 2-theta of about 18.86 degrees, about 19.28 degrees, and about 23.12 degrees. In some embodiments, Pattern B is characterized by an XRPD pattern comprising peaks at angles 2-theta of about 16.98 degrees, about 18.86 degrees, about 19.28 degrees, about 23.12 degrees, and about 25.53 degrees.

In some embodiments, Pattern B is characterized by an XRPD pattern comprising:
a. peaks at angles 2-theta of 9.42±0.2 degrees and 14.52±0.2 degrees;
b. peaks at angles 2-theta of 9.42±0.2 degrees, 13.42±0.2 degrees, and 14.52±0.2 degrees; or
c. peaks at angles 2-theta of 3.61±0.2 degrees, 9.42±0.2 degrees, 13.16±0.2 degrees, 13.42±0.2 degrees, and 14.52±0.2 degrees. In some embodiments, Pattern B is characterized by an XRPD pattern comprising peaks at angles 2-theta of 9.42±0.2 degrees and 14.52±0.2 degrees. In some embodiments, Pattern B is characterized by an XRPD pattern comprising peaks at angles 2-theta of 9.42±0.2 degrees, 13.42±0.2 degrees, and 14.52±0.2 degrees. In some embodiments, Pattern B is characterized by an XRPD pattern comprising peaks at angles 2-theta of 3.61±0.2 degrees, 9.42±0.2 degrees, 13.16±0.2 degrees, 13.42±0.2 degrees, and 14.52±0.2 degrees.

In some embodiments, Pattern B is characterized by an XRPD pattern comprising:
a. peaks at angles 2-theta of about 9.42 degrees and about 14.52 degrees;
b. peaks at angles 2-theta of about 9.42 degrees, about 13.42 degrees, and about 14.52 degrees; or
c. peaks at angles 2-theta of about 3.61 degrees, about 9.42 degrees, about 13.16 degrees, about 13.42 degrees, and about 14.52 degrees. In some embodiments, Pattern B is characterized by an XRPD pattern comprising peaks at angles 2-theta of about 9.42 degrees and about 14.52 degrees. In some embodiments, Pattern B is characterized by an XRPD pattern comprising peaks at angles 2-theta of about 9.42 degrees, about 13.42 degrees, and about 14.52 degrees. In some embodiments, Pattern B is characterized by an XRPD pattern comprising peaks at angles 2-theta of about 3.61 degrees, about 9.42 degrees, about 13.16 degrees, about 13.42 degrees, and about 14.52 degrees.

In some embodiments, Pattern B is characterized by an XRPD pattern comprising peaks at angles 2-theta of 3.61±0.2 degrees, 7.34±0.2 degrees, 9.42±0.2 degrees, 15.76±0.2 degrees, and 23.12±0.2 degrees. In some embodiments, Pattern B is characterized by an XRPD pattern comprising peaks at angles 2-theta of about 3.61 degrees, about 7.34 degrees, about 9.42 degrees, about 15.76 degrees, and about 23.12 degrees.

In some embodiments, Pattern B has an XRPD pattern comprising one or more (e.g., at least one, at least two, at least three, at least four, at least five, at least six, at least seven, at least eight, at least nine, at least ten, at least eleven, or at least twelve) of the peaks at angles 2-theta of 3.61±0.2 degrees, 7.34±0.2 degrees, 9.42±0.2 degrees, 13.16±0.2 degrees, 13.42±0.2 degrees, 14.52±0.2 degrees, 15.76±0.2 degrees, 16.98±0.2 degrees, 18.86±0.2 degrees, 19.28±0.2 degrees, 23.12±0.2 degrees, or 25.53±0.2 degrees. In some embodiments, Pattern B has an XRPD pattern comprising one or more (e.g., at least one, at least two, at least three, at least four, at least five, at least six, at least seven, at least eight, at least nine, at least ten, at least eleven, or at least twelve) of the peaks at angles 2-theta of about 3.61 degrees, about 7.34 degrees, about 9.42 degrees, about 13.16 degrees, about 13.42 degrees, about 14.52 degrees, about 15.76 degrees, about 16.98 degrees, about 18.86 degrees, about 19.28 degrees, about 23.12 degrees, or about 25.53 degrees.

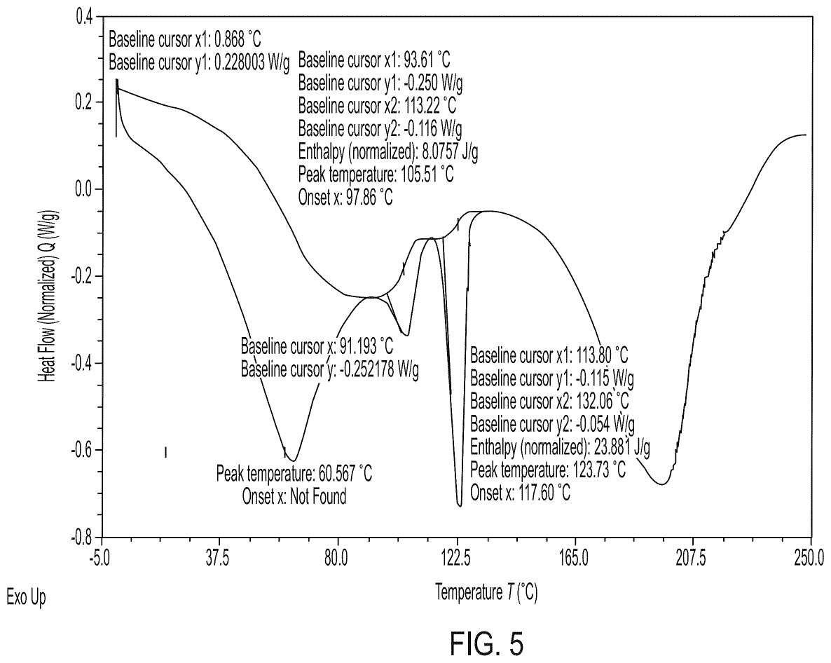

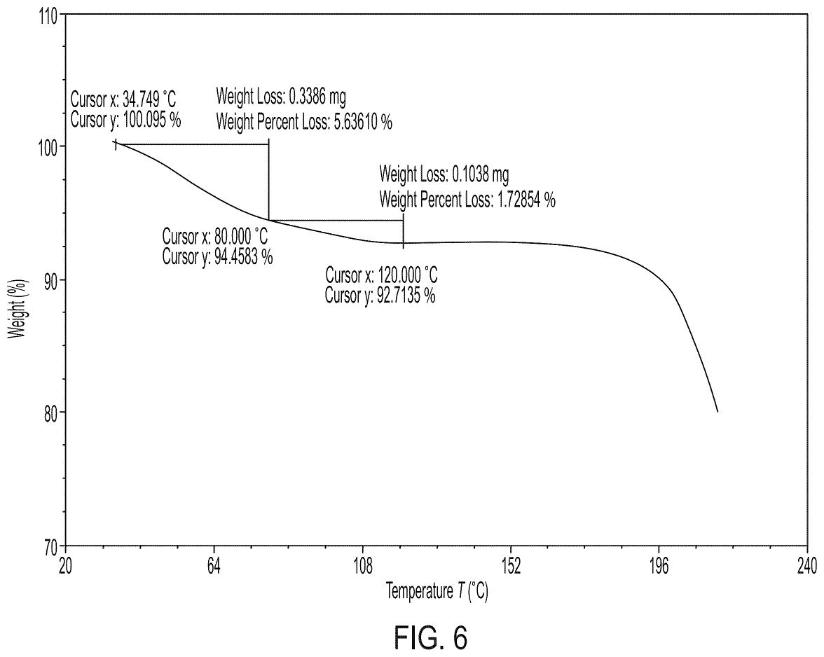

In some embodiments, Pattern B has a DSC graph substantially as shown in FIG. 5. In some embodiments, Pattern B is characterized by an endotherm peak between about 55° C. and about 66° C., and an endotherm peak between about 119° C. and about 129° C., as determined by DSC. In some embodiments, Pattern B is characterized by an endotherm peak between about 58° C. and about 63° C., and an endotherm peak between about 121° C. and about 127° C., as determined by DSC. In some embodiments, Pattern B is characterized by endotherm peaks at about 60.5° C. and about 123.7° C., as determined by DSC. In some embodiments, Pattern B has a TGA graph substantially as shown in FIG. 6. In some embodiments, Pattern B is characterized by a weight loss of about 2% to about 9% after heating from a temperature between about 30° C. and about 40° C. to a temperature between about 75° C. to about 85° C., as determined by TGA. In some embodiments, Pattern B is characterized by a weight loss of about 4% to about 7% after heating from a temperature between about 32° C. and about 36° C. to a temperature between about 78° C. to about 82° C., as determined by TGA. In some embodiments, Pattern B is characterized by a weight loss of about 5.6% after heating from about 34° C. to about 80° C., as determined by TGA.

In some embodiments of Pattern B, at least one, at least two, or all of the following (a)-(c) apply:
(a) Pattern B has an XRPD pattern substantially as shown in FIG. 4;
(b) Pattern B has a DSC graph substantially as shown in FIG. 5;
(c) Pattern B has a TGA graph substantially as shown in FIG. 6.

In some embodiments, (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B is a solvate. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B is a hydrate. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B is a channel hydrate. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B contains about 3.5 equivalents (7.4% by weight) of water according to KF analysis.

Pattern B may be obtained from water/ethanol (EtOH) mixture, water/IPA mixture and water/acetone mixture by anti-solvent addition experiments. In some embodiments, Pattern B shows a broad dehydration peak from about 1° C. as measured by DSC. In some embodiments, Pattern B shows dehydration upon melting from about 94° C. as measured by DSC. In some embodiments, Pattern B shows about 5.6% weight loss at about 80° C. as measured by TGA. In some embodiments, Pattern B shows about 5.6% weight loss at about 80° C. and about 1.7% weight loss from 80° C. to 120° C. as measured by TGA. In some embodiments, ¹H-NMR for Pattern B shows stoichiometry of (S)-4-((2-methoxyethyl)(4-(5,6,7,8-tetrahydro-1,8-naphthyridin-2-yl)butyl)amino)-2-(quinazolin-4-ylamino)butanoic acid free form: L-tartaric acid is 1:2.0 on a molar basis and about 0.3% IPA residue by weight (0.05 equiv. by molar ratio). In some embodiments, Pattern B shows a reversible hydration-dehydration behavior.

### Pattern C Crystalline Form

In some embodiments, provided herein is a crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid designated as Pattern C. A method of making Pattern C is shown in Example 5.

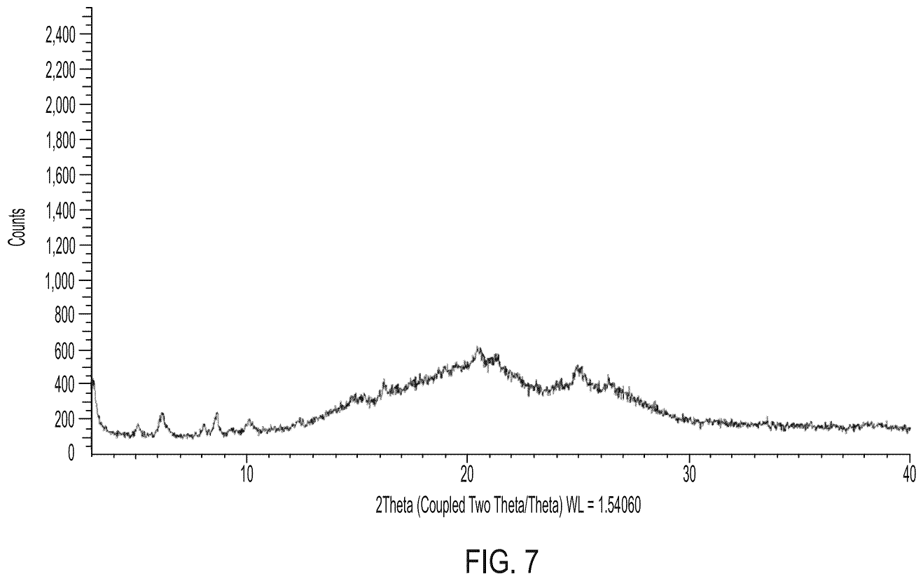

In some embodiments, Pattern C has an XRPD pattern substantially as shown in FIG. 7. In some embodiments, Pattern C has an XRPD pattern comprising one or more (e.g., at least one, at least two, at least three, at least four, at least five, at least six, at least seven, at least eight, at least nine, or at least ten) of the peaks at angles 2-theta corresponding to the peaks of greatest relative intensities in the XRPD pattern substantially as shown in FIG. 7. In some embodiments, Pattern C has an XRPD pattern comprising one or more (e.g., at least one, at least two, at least three, at least four, at least five, at least six, at least seven, at least eight, at least nine, or at least ten) of the peaks at angles 2-theta of 6.21±0.2 degrees, 8.68±0.2 degrees, 15.36±0.2 degrees, 16.05±0.2 degrees, 16.24±0.2 degrees, 20.45±0.2 degrees, 21.26±0.2 degrees, 24.97±0.2 degrees, 26.43±0.2 degrees, or 35.53±0.2 degrees. In some embodiments, Pattern C has an XRPD pattern comprising one or more (e.g., at least one, at least two, at least three, at least four, at least five, at least six, at least seven, at least eight, at least nine, or at least ten) of the peaks at angles 2-theta of about 6.21 degrees, about 8.68 degrees, about 15.36 degrees, about 16.05 degrees, about 16.24 degrees, about 20.45 degrees, about 21.26 degrees, about 24.97 degrees, about 26.43 degrees, or about 35.53 degrees. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Pattern C, can independently vary by +0.6 degrees, +0.4 degrees, +0.2 degrees, or +0.1 degrees 2-theta. In some embodiments, each peak assignment listed herein, including for Pattern C, can independently vary by ±0.2 degrees 2-theta.

In some embodiments, Pattern C has an XRPD pattern comprising one or more (e.g., at least one, at least two, at least three, at least four, at least five, at least six, at least seven, at least eight, at least nine, or at least ten) of the peaks at angles 2-theta corresponding to the peaks of greatest relative intensities in the XRPD pattern substantially as shown in Table 3. In some embodiments, Pattern C has an XRPD pattern comprising one or more (e.g., at least one, at least two, at least three, at least four, at least five, at least six, at least seven, at least eight, at least nine, or at least ten) of the peaks at angles 2-theta of 6.21±0.2 degrees, 8.68±0.2 degrees, 15.36±0.2 degrees, 16.05±0.2 degrees, 16.24±0.2 degrees, 20.45±0.2 degrees, 21.26±0.2 degrees, 24.97±0.2 degrees, 26.43±0.2 degrees, or 35.53±0.2 degrees. In some embodiments, Pattern C has an XRPD pattern comprising one or more (e.g., at least one, at least two, at least three, at least four, at least five, at least six, at least seven, at least eight, at least nine, or at least ten) of the peaks at angles 2-theta of about 6.21 degrees, about 8.68 degrees, about 15.36 degrees, about 16.05 degrees, about 16.24 degrees, about 20.45 degrees, about 21.26 degrees, about 24.97 degrees, about 26.43 degrees, or about 35.53 degrees. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Pattern C, can independently vary by +0.6 degrees, +0.4 degrees, +0.2 degrees, or +0.1 degrees 2-theta. In some embodiments, each peak assignment listed herein, including for Pattern C, can independently vary by +0.2 degrees 2-theta.

TABLE 3

| | | Angle | d | Rel. | |
|---|---|---|---|---|---|
| | | 2-Theta | Value | Intensity | |
| Index | Name | (degrees) | (Å) | (%)* | Intensity |
| 3 | Peak#3 | 8.115 | 10.88634 | 14.70 | 141.26 |
| 1 | Peak#1 | 5.118 | 17.25292 | 21.10 | 145.015 |
| 5 | Peak#5 | 10.143 | 8.71412 | 25.90 | 176.447 |
| 13 | Peak#13 | 35.531 | 2.52454 | 16.80 | 180.716 |
| 2 | Peak#2 | 6.209 | 14.22268 | 44.80 | 186.905 |
| 4 | Peak#4 | 8.676 | 10.18408 | 44.00 | 200.354 |
| 6 | Peak#6 | 15.361 | 5.76348 | 32.20 | 325.398 |
| 7 | Peak#7 | 16.047 | 5.51879 | 37.60 | 354.942 |
| 8 | Peak#8 | 16.241 | 5.45335 | 37.20 | 359.403 |
| 12 | Peak#12 | 26.426 | 3.37005 | 44.50 | 381.761 |
| 11 | Peak#11 | 24.972 | 3.56288 | 66.00 | 450.439 |
| 10 | Peak#10 | 21.264 | 4.17511 | 88.20 | 519.994 |
| 9 | Peak#9 | 20.446 | 4.34019 | 100.00 | 538.718 |

*Relative intensity is calculated based on net peak intensity. Net peak intensity is calculated after background removal from peak intensity.

In some embodiments, Pattern C is characterized by an XRPD pattern comprising peaks at angles 2-theta of 5.12±0.2 degrees and 6.21±0.2 degrees.

In some embodiments, Pattern C is characterized by an XRPD pattern comprising peaks at angles 2-theta of about 5.12 degrees and about 6.21 degrees.

In some embodiments, Pattern C is characterized by an XRPD pattern comprising:
a. peaks at angles 2-theta of 20.45±0.2 degrees and 21.26±0.2 degrees;
b. peaks at angles 2-theta of 20.45±0.2 degrees, 21.26±0.2 degrees, and 24.97±0.2 degrees; or
c. peaks at angles 2-theta of 6.21±0.2 degrees, 20.45±0.2 degrees, 21.26±0.2 degrees, 24.97±0.2 degrees, and 26.43±0.2 degrees. In some embodiments, Pattern C is characterized by an XRPD pattern comprising peaks at angles 2-theta of 20.45±0.2 degrees and 21.26±0.2 degrees. In some embodiments, Pattern C is characterized by an XRPD pattern comprising peaks at angles 2-theta of 20.45±0.2 degrees, 21.26±0.2 degrees, and 24.97±0.2 degrees. In some embodiments, Pattern C is characterized by an XRPD pattern comprising peaks at angles 2-theta of 6.21±0.2 degrees, 20.45±0.2 degrees, 21.26±0.2 degrees, 24.97±0.2 degrees, and 26.43±0.2 degrees.

In some embodiments, Pattern C is characterized by an XRPD pattern comprising:
a. peaks at angles 2-theta of about 20.45 degrees and about 21.26 degrees;
b. peaks at angles 2-theta of about 20.45 degrees, about 21.26 degrees, and about 24.97 degrees; or
c. peaks at angles 2-theta of about 6.21 degrees, about 20.45 degrees, about 21.26 degrees, about 24.97 degrees, and about 26.43 degrees. In some embodiments, Pattern C is characterized by an XRPD pattern comprising peaks at angles 2-theta of about 20.45 degrees and about 21.26 degrees. In some embodiments, Pattern C is characterized by an XRPD pattern comprising peaks at angles 2-theta of about 20.45 degrees, about 21.26 degrees, and about 24.97 degrees. In some embodiments, Pattern C is characterized by an XRPD pattern comprising peaks at angles 2-theta of about 6.21 degrees, about 20.45 degrees, about 21.26 degrees, about 24.97 degrees, and about 26.43 degrees.

In some embodiments, Pattern C is characterized by an XRPD pattern comprising:
a. peaks at angles 2-theta of 6.21±0.2 degrees and 8.68±0.2 degrees;
b. peaks at angles 2-theta of 6.21±0.2 degrees, 8.68±0.2 degrees, and 10.14±0.2 degrees; or
c. peaks at angles 2-theta of 5.12±0.2 degrees, 6.21±0.2 degrees, 8.12±0.2 degrees, 8.68±0.2 degrees, and 10.14±0.2 degrees. In some embodiments, Pattern C is characterized by an XRPD pattern comprising peaks at angles 2-theta of 6.21±0.2 degrees and 8.68±0.2 degrees. In some embodiments, Pattern C is characterized by an XRPD pattern comprising peaks at angles 2-theta of 6.21±0.2 degrees, 8.68±0.2 degrees, and 10.14±0.2 degrees. In some embodiments, Pattern C is characterized by an XRPD pattern comprising peaks at angles 2-theta of 5.12±0.2 degrees, 6.21±0.2 degrees, 8.12±0.2 degrees, 8.68±0.2 degrees, and 10.14±0.2 degrees.

In some embodiments, Pattern C is characterized by an XRPD pattern comprising:
a. peaks at angles 2-theta of about 6.21 degrees and about 8.68 degrees;
b. peaks at angles 2-theta of about 6.21 degrees, about 8.68 degrees, and about 10.14 degrees; or
c. peaks at angles 2-theta of about 5.12 degrees, about 6.21 degrees, about 8.12 degrees, about 8.68 degrees, and about 10.14 degrees. In some embodiments, Pattern C is characterized by an XRPD pattern comprising peaks at angles 2-theta of about 6.21 degrees and about 8.68 degrees. In some embodiments, Pattern C is characterized by an XRPD pattern comprising peaks at angles 2-theta of about 6.21 degrees, about 8.68 degrees, and about 10.14 degrees. In some embodiments, Pattern C is characterized by an XRPD pattern comprising peaks at angles 2-theta of about 5.12 degrees, about 6.21 degrees, about 8.12 degrees, about 8.68 degrees, and about 10.14 degrees.

In some embodiments, Pattern C has an XRPD pattern comprising one or more (e.g., at least one, at least two, at least three, at least four, at least five, at least six, at least seven, at least eight, or at least nine) of the peaks at angles 2-theta of 5.12±0.2 degrees, 6.21±0.2 degrees, 8.12±0.2 degrees, 8.68±0.2 degrees, 10.14±0.2 degrees, 20.45±0.2 degrees, 21.26±0.2 degrees, 24.97±0.2 degrees, or 26.43±0.2 degrees. In some embodiments, Pattern C has an XRPD pattern comprising one or more (e.g., at least one, at least two, at least three, at least four, at least five, at least six, at least seven, at least eight, or at least nine) of the peaks at angles 2-theta of about 5.12 degrees, about 6.21 degrees, about 8.12 degrees, about 8.68 degrees, about 10.14 degrees, about 20.45 degrees, about 21.26 degrees, about 24.97 degrees, or about 26.43 degrees.

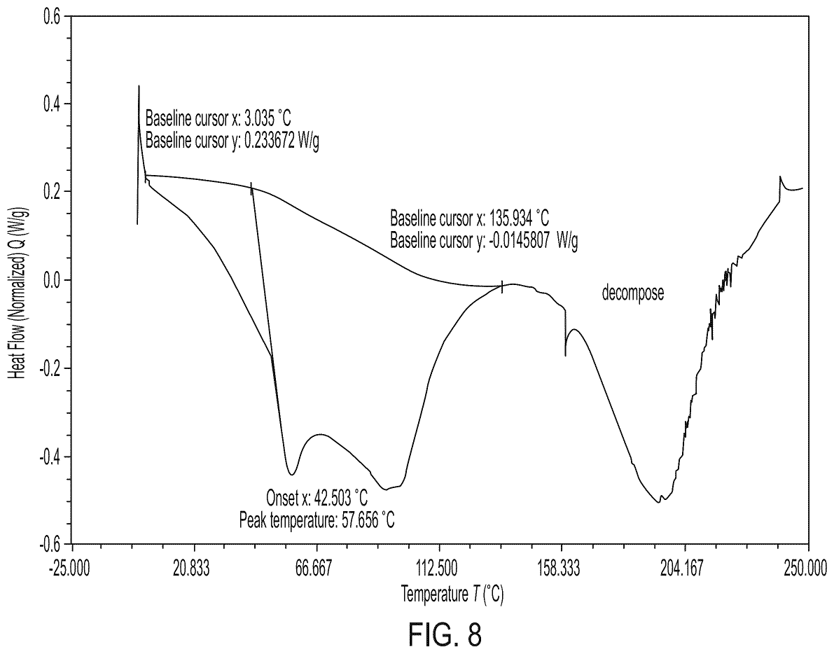

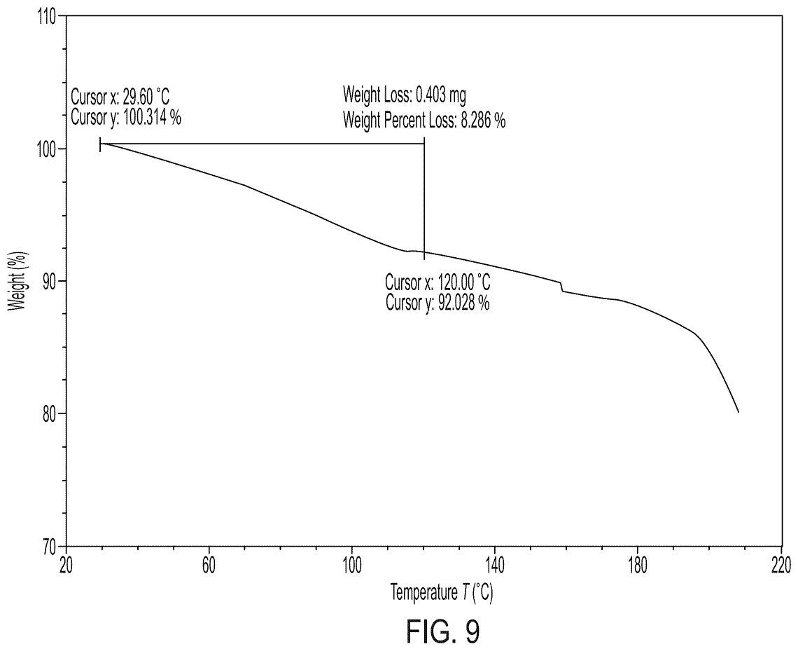

In some embodiments, Pattern C has a DSC graph substantially as shown in FIG. 8. In some embodiments, Pattern C is characterized by an endotherm peak between about 50° C. and about 65° C., as determined by DSC. In some embodiments, Pattern C is characterized by an endotherm peak between about 55° C. and about 61° C., as determined by DSC. In some embodiments, Pattern C is characterized by an endotherm peak at about 58° C., as determined by DSC. In some embodiments, Pattern C has a TGA graph substantially as shown in FIG. 9. In some embodiments, Pattern C is characterized by a weight loss of about 4% to about 12% after heating from a temperature between about 25° C. and about 35° C. to a temperature between about 115° C. to about 125° C., as determined by TGA. In some embodiments, Pattern C is characterized by a weight loss of about 6% to about 10% after heating from a temperature between about 28° C. and about 32° C. to a temperature between about 118° C. to about 122° C., as determined by TGA. In some embodiments, Pattern C is characterized by a weight loss of about 8.3% after heating from about 30° C. to about 120° C., as determined by TGA.

In some embodiments of Pattern C, at least one, at least two, or all of the following (a)-(c) apply:
(a) Pattern C has an XRPD pattern substantially as shown in FIG. 7;
(b) Pattern C has a DSC graph substantially as shown in FIG. 8;
(c) Pattern C has a TGA graph substantially as shown in FIG. 9.

Pattern C may be obtained from an IPA/water mixture by a slow cooling experiment and from a water/acetone mixture by a vapor diffusion experiment. In some embodiments, Pattern C shows an endothermic peak from about 3° C. as measured by DSC. In some embodiments, TGA for Pattern C shows about 8.3% weight loss at about 120° C. as measured by TGA. In some embodiments, ¹H-NMR for Pattern C shows stoichiometry of (S)-4-((2-methoxyethyl)(4-(5,6,7,8-tetrahydro-1,8-naphthyridin-2-yl)butyl)amino)-2-(quinazolin-4-ylamino)butanoic acid free form: L-tartaric acid is 1:1.8 and about 3.5% IPA residue by weight (0.5 equiv. by molar ratio). In some embodiments, Pattern C converts to Pattern B in IPA/water mixture by equilibration at 5° C. after 3 days.

Pattern A was shown to be thermodynamically stable based on water activity experiments. In a test of stability under different conditions of water activity (0, 0.2, 0.4, 0.6, 0.8, and 1.0 water activity), Pattern A was stable under most conditions. As shown in Example 9 and Table 6, when samples of Pattern A and Pattern B were placed in methanol/water solutions of varying water activity, Pattern A was obtained when the water activity was ≤0.8 at 5° C., when the water activity was ≤0.8 at 25° C., and when the water activity was ≤1.0 at 50° C. Pattern B was only obtained under conditions when the water activity was 1.0 at 5° C. and 25° C.

Additionally, the Pattern A(S)-4-((2-methoxyethyl)(4-(5,6,7,8-tetrahydro-1,8-naphthyridin-2-yl)butyl)amino)-2-(quinazolin-4-ylamino)butanoic acid-tartaric acid cocrystal was shown to have good stability under stress conditions. As shown in Example 10A and Table 7, in an open container, the Pattern A tartaric acid cocrystal was stable at 25° C. and 92.5% relative humidity and at 40° C. and 75% RH for up to four weeks.

In some embodiments of a crystalline form disclosed herein (e.g., Pattern A, Pattern B, or Pattern C), the crystalline form is substantially anhydrous. In some embodiments of a crystalline form disclosed herein (e.g., Pattern A, Pattern B, or Pattern C), the crystalline form is a hydrate. For example, in some embodiments, the crystalline form has a water content of less than about 1%, about 0.5%, or about 0.1% by weight. In some embodiments, the crystalline form has a water content in % by weight of one of about 1, 1.5, 2, 2.5, 3, 3.5, 4, 4.5 5, 5.5, 6, 6.5, 7, 7.5 8, 8.5, 9, 9.5, 10, 10.5, 11, 11.5, 12, 12.5, 13, 13.5, 14, 14.5, or 15, or a range between any two of the preceding values. In some embodiments, the crystalline form has a water content in % by weight of one of about 1, 1.5, 2, 2.5, 3, 3.5, 4, 4.5 5, 5.5, 6, 6.5, 7, 7.5 8, 8.5, 9, 9.5, or 10, or a range between any two of the preceding values. For example, in some embodiments, the water content is about 1-15 wt %. In some embodiments, the water content is about 2-15 wt %. In some embodiments, the water content is about 3-15 wt %. In some embodiments, the water content is about 4-15 wt %. In some embodiments, the water content is about 5-15 wt %. In some embodiments, the water content is about 6-15 wt %. In some embodiments, the water content is about 7-15 wt %. In some embodiments, the water content is about 8-15 wt %. In some embodiments, the water content is about 9-15 wt %. In some embodiments, the water content is about 1-12 wt %. In some embodiments, the water content is about 2-12 wt %. In some embodiments, the water content is about 3-12 wt %. In some embodiments, the water content is about 4-12 wt %. In some embodiments, the water content is about 5-12 wt %. In some embodiments, the water content is about 6-12 wt %. In some embodiments, the water content is about 7-12 wt %. In some embodiments, the water content is about 8-12 wt %. In some embodiments, the water content is about 9-12 wt %. In some embodiments, the water content is about 1-10 wt %. In some embodiments, the water content is about 2-10 wt %. In some embodiments, the water content is about 3-10 wt %. In some embodiments, the water content is about 4-10 wt %. In some embodiments, the water content is about 5-10 wt %. In some embodiments, the water content is about 6-10 wt %. In some embodiments, the water content is about 7-10 wt %. In some embodiments, the water content is about 8-10 wt %. In some embodiments, the water content is about 9-10 wt %. In some embodiments, the water content is about 1-9 wt %. In some embodiments, the water content is about 2-9 wt %. In some embodiments, the water content is about 3-9 wt %. In some embodiments, the water content is about 4-9 wt %. In some embodiments, the water content is about 5-9 wt %. In some embodiments, the water content is about 6-9 wt %. In some embodiments, the water content is about 7-9 wt %. In some embodiments, the water content is about 8-9 wt %. In some embodiments, the water content is about 1-8 wt %. In some embodiments, the water content is about 2-8 wt %. In some embodiments, the water content is about 3-8 wt %. In some embodiments, the water content is about 4-8 wt %. In some embodiments, the water content is about 5-8 wt %. In some embodiments, the water content is about 6-8 wt %. In some embodiments, the water content is about 7-8 wt %. In some embodiments, the water content is about 1-7 wt %. In some embodiments, the water content is about 2-7 wt %. In some embodiments, the water content is about 3-7 wt %. In some embodiments, the water content is about 4-7 wt %. In some embodiments, the water content is about 5-7 wt %. In some embodiments, the water content is about 6-7 wt %. In some embodiments, the water content is about 1-6 wt %. In some embodiments, the water content is about 2-6 wt %. In some embodiments, the water content is about 3-6 wt %. In some embodiments, the water content is about 4-6 wt %. In some embodiments, the water content is about 5-6 wt %. In some embodiments, the water content is about 1-5 wt %. In some embodiments, the water content is about 2-5 wt %. In some embodiments, the water content is about 3-5 wt %. In some embodiments, the water content is about 4-5 wt %. In some embodiments, the water content is about 1-4 wt %. In some embodiments, the water content is about 2-4 wt %. In some embodiments, the water content is about 3-4 wt %. In some embodiments, the crystalline form has a water content that varies in % by weight of +1, +0.75, +0.5, ±0.25, or +0.1 around a % by weight of one of 1, 1.5, 2, 2.5, 3, 3.5, 4, 4.5 5, 5.5, 6, 6.5, 7, 7.5 8, 8.5, 9, 9.5, or 10. For example, in some embodiments, the water content of the crystalline form is about 1±0.75 wt %. In some embodiments, the water content is about 1±0.5 wt %. In some embodiments, the water content is about 1±0.25 wt %. In some embodiments, the water content is about 1±0.1 wt %. In some embodiments, the water content is about 1.5±1 wt %. In some embodiments, the water content is about 1.5±0.75 wt %. In some embodiments, the water content is about 1.5±0.5 wt %. In some embodiments, the water content is about 1.5±0.25 wt %. In some embodiments, the water content is about 1.5±0.1 wt %. In some embodiments, the water content is about 2±1 wt %. In some embodiments, the water content is about 2±0.75 wt %. In some embodiments, the water content is about 2±0.5 wt %. In some embodiments, the water content is about 2±0.25 wt %. In some embodiments, the water content is about 2±0.1 wt %. In some embodiments, the water content is about 2.5±1 wt %. In some embodiments, the water content is about 2.5±0.75 wt %. In some embodiments, the water content is about 2.5±0.5 wt %. In some embodiments, the water content is about 2.5±0.25 wt %. In some embodiments, the water content is about 2.5±0.1 wt %. In some embodiments, the water content is about 3±1 wt %. In some embodiments, the water content is about 3±0.75 wt %. In some embodiments, the water content is about 3±0.5 wt %. In some embodiments, the water content is about 3±0.25 wt %. In some embodiments, the water content is about 3±0.1 wt %. In some embodiments, the water content is about 3.5±1 wt %. In some embodiments, the water content is about 3.5±0.75 wt %. In some embodiments, the water content is about 3.5±0.5 wt %. In some embodiments, the water content is about 3.5±0.25 wt %. In some embodiments, the water content is about 3.5±0.1 wt %. In some embodiments, the water content is about 4±1 wt %. In some embodiments, the water content is about 4±0.75 wt %. In some embodiments, the water content is about 4±0.5 wt %. In some embodiments, the water content is about 4±0.25 wt %. In some embodiments, the water content is about 4±0.1 wt %. In some embodiments, the water content is about 4.5±1 wt %. In some embodiments, the water content is about 4.5±0.75 wt %. In some embodiments, the water content is about 4.5±0.5 wt %. In some embodiments, the water content is about 4.5±0.25 wt %. In some embodiments, the water content is about 4.5±0.1 wt %. In some embodiments, the water content is about 5±1 wt %. In some embodiments, the water content is about 5±0.75 wt %. In some embodiments, the water content is about 5±0.5 wt %. In some embodiments, the water content is about 5±0.25 wt %. In some embodiments, the water content is about 5±0.1 wt %. In some embodiments, the water content is about 5.5±1 wt %. In some embodiments, the water content is about 5.5±0.75 wt %. In some embodiments, the water content is about 5.5±0.5 wt %. In some embodiments, the water content is about 5.5±0.25 wt %. In some embodiments, the water content is about 5.5±0.1 wt %. In some embodiments, the water content is about 6±1 wt %. In some embodiments, the water content is about 6±0.5 wt %. In some embodiments, the water content is about 6.5±1 wt %. In some embodiments, the water content is about 6.5±0.5 wt %. In some embodiments, the water content is about 7±1 wt %. In some embodiments, the water content is about 7±0.5 wt %. In some embodiments, the water content is about 7.5±1 wt %. In some embodiments, the water content is about 7.5±0.5 wt %. In some embodiments, the water content is about 8±1 wt %. In some embodiments, the water content is about 8±0.5 wt %. In some embodiments, the water content is about 8.5±1 wt %. In some embodiments, the water content is about 8.5±0.5 wt %. In some embodiments, the water content is about 9±1 wt %. In some embodiments, the water content is about 9±0.5 wt %. In some embodiments, the water content is about 9.5±0.5 wt %. In some embodiments, the water content is about 9.5±0.25 wt %. In some embodiments, the water content is about 10±1 wt %. In some embodiments, the water content is about 10±0.5 wt %. In some embodiments, the water content is about 10±0.25 wt %. In some embodiments, the water content is about 11±1 wt %. In some embodiments, the water content is about 11±0.5 wt %. In some embodiments, the water content is about 11±0.25 wt %. In some embodiments, the water content is about 12±1 wt %. In some embodiments, the water content is about 12±0.5 wt %. In some embodiments, the water content is about 12±0.25 wt %. In some embodiments, the water content is about 13±1 wt %. In some embodiments, the water content is about 13±0.5 wt %. In some embodiments, the water content is about 13±0.25 wt %. In some embodiments, the water content is about 14±1 wt %. In some embodiments, the water content is about 14±0.5 wt %. In some embodiments, the water content is about 14±0.25 wt %. In some embodiments, the water content is about 15±1 wt %. In some embodiments, the water content is about 15±0.5 wt %. In some embodiments, the water content is about 15±0.25 wt %.

In some embodiments of a method of preparing a crystalline form disclosed herein (e.g., Pattern A, Pattern B, or Pattern C), the method can comprise preparing (e.g., drying) the crystalline form such that the crystalline form is substantially anhydrous. For example, in some embodiments, the method further comprises preparing the crystalline form such that the crystalline form has a water content of less than about 1%, about 0.5%, or about 0.1% by weight. In some embodiments, the method further comprises preparing the crystalline form such that the crystalline form has a water content in % by weight of one of about 1, 1.5, 2, 2.5, 3, 3.5, 4, 4.5 5, 5.5, 6, 6.5, 7, 7.5 8, 8.5, 9, 9.5, 10, 10.5, 11, 11.5, 12, 12.5, 13, 13.5, 14, 14.5, or 15, or a range between any two of the preceding values. In some embodiments, the method further comprises preparing the crystalline form such that the crystalline form has a water content in % by weight of one of about 1, 1.5, 2, 2.5, 3, 3.5, 4, 4.5 5, 5.5, 6, 6.5, 7, 7.5 8, 8.5, 9, 9.5, or 10, or a range between any two of the preceding values. In some embodiments, the method comprises a humidification step. For example, in some embodiments of the crystalline form, the water content is about 1-15 wt %. In some embodiments, the water content is about 2-15 wt %. In some embodiments, the water content is about 3-15 wt %. In some embodiments, the water content is about 4-15 wt %. In some embodiments, the water content is about 5-15 wt %. In some embodiments, the water content is about 6-15 wt %. In some embodiments, the water content is about 7-15 wt %. In some embodiments, the water content is about 8-15 wt %. In some embodiments, the water content is about 9-15 wt %. In some embodiments, the water content is about 1-12 wt %. In some embodiments, the water content is about 2-12 wt %. In some embodiments, the water content is about 3-12 wt %. In some embodiments, the water content is about 4-12 wt %. In some embodiments, the water content is about 5-12 wt %. In some embodiments, the water content is about 6-12 wt %. In some embodiments, the water content is about 7-12 wt %. In some embodiments, the water content is about 8-12 wt %. In some embodiments, the water content is about 9-12 wt %. In some embodiments, the water content is about 1-10 wt %. In some embodiments, the water content is about 2-10 wt %. In some embodiments, the water content is about 3-10 wt %. In some embodiments, the water content is about 4-10 wt %. In some embodiments, the water content is about 5-10 wt %. In some embodiments, the water content is about 6-10 wt %. In some embodiments, the water content is about 7-10 wt %. In some embodiments, the water content is about 8-10 wt %. In some embodiments, the water content is about 9-10 wt %. In some embodiments, the water content is about 1-9 wt %. In some embodiments, the water content is about 2-9 wt %. In some embodiments, the water content is about 3-9 wt %. In some embodiments, the water content is about 4-9 wt %. In some embodiments, the water content is about 5-9 wt %. In some embodiments, the water content is about 6-9 wt %. In some embodiments, the water content is about 7-9 wt %. In some embodiments, the water content is about 8-9 wt %. In some embodiments, the water content is about 1-8 wt %. In some embodiments, the water content is about 2-8 wt %. In some embodiments, the water content is about 3-8 wt %. In some embodiments, the water content is about 4-8 wt %. In some embodiments, the water content is about 5-8 wt %. In some embodiments, the water content is about 6-8 wt %. In some embodiments, the water content is about 7-8 wt %. In some embodiments, the water content is about 1-7 wt %. In some embodiments, the water content is about 2-7 wt %. In some embodiments, the water content is about 3-7 wt %. In some embodiments, the water content is about 4-7 wt %. In some embodiments, the water content is about 5-7 wt %. In some embodiments, the water content is about 6-7 wt %. In some embodiments, the water content is about 1-6 wt %. In some embodiments, the water content is about 2-6 wt %. In some embodiments, the water content is about 3-6 wt %. In some embodiments, the water content is about 4-6 wt %. In some embodiments, the water content is about 5-6 wt %. In some embodiments, the water content is about 1-5 wt %. In some embodiments, the water content is about 2-5 wt %. In some embodiments, the water content is about 3-5 wt %. In some embodiments, the water content is about 4-5 wt %. In some embodiments, the water content is about 1-4 wt %. In some embodiments, the water content is about 2-4 wt %. In some embodiments, the water content is about 3-4 wt %. In some embodiments, the crystalline form has a water content that varies in % by weight of +1, +0.75, +0.5, ±0.25, or +0.1 around a % by weight of one of 1, 1.5, 2, 2.5, 3, 3.5, 4, 4.5 5, 5.5, 6, 6.5, 7, 7.5 8, 8.5, 9, 9.5, or 10. For example, in some embodiments, the water content of the crystalline form is about 1±0.75 wt %. In some embodiments, the water content is about 1±0.5 wt %. In some embodiments, the water content is about 1±0.25 wt %. In some embodiments, the water content is about 1±0.1 wt %. In some embodiments, the water content is about 1.5±1 wt %. In some embodiments, the water content is about 1.5±0.75 wt %. In some embodiments, the water content is about 1.5±0.5 wt %. In some embodiments, the water content is about 1.5±0.25 wt %. In some embodiments, the water content is about 1.5±0.1 wt %. In some embodiments, the water content is about 2±1 wt %. In some embodiments, the water content is about 2±0.75 wt %. In some embodiments, the water content is about 2±0.5 wt %. In some embodiments, the water content is about 2±0.25 wt %. In some embodiments, the water content is about 2±0.1 wt %. In some embodiments, the water content is about 2.5±1 wt %. In some embodiments, the water content is about 2.5±0.75 wt %. In some embodiments, the water content is about 2.5±0.5 wt %. In some embodiments, the water content is about 2.5±0.25 wt %. In some embodiments, the water content is about 2.5±0.1 wt %. In some embodiments, the water content is about 3±1 wt %. In some embodiments, the water content is about 3±0.75 wt %. In some embodiments, the water content is about 3±0.5 wt %. In some embodiments, the water content is about 3±0.25 wt %. In some embodiments, the water content is about 3±0.1 wt %. In some embodiments, the water content is about 3.5±1 wt %. In some embodiments, the water content is about 3.5±0.75 wt %. In some embodiments, the water content is about 3.5±0.5 wt %. In some embodiments, the water content is about 3.5±0.25 wt %. In some embodiments, the water content is about 3.5±0.1 wt %. In some embodiments, the water content is about 4±1 wt %. In some embodiments, the water content is about 4±0.75 wt %. In some embodiments, the water content is about 4±0.5 wt %. In some embodiments, the water content is about 4±0.25 wt %. In some embodiments, the water content is about 4±0.1 wt %. In some embodiments, the water content is about 4.5±1 wt %. In some embodiments, the water content is about 4.5±0.75 wt %. In some embodiments, the water content is about 4.5±0.5 wt %. In some embodiments, the water content is about 4.5±0.25 wt %. In some embodiments, the water content is about 4.5±0.1 wt %. In some embodiments, the water content is about 5±1 wt %. In some embodiments, the water content is about 5±0.75 wt %. In some embodiments, the water content is about 5±0.5 wt %. In some embodiments, the water content is about 5±0.25 wt %. In some embodiments, the water content is about 5±0.1 wt %. In some embodiments, the water content is about 5.5±1 wt %. In some embodiments, the water content is about 5.5±0.75 wt %. In some embodiments, the water content is about 5.5±0.5 wt %. In some embodiments, the water content is about 5.5±0.25 wt %. In some embodiments, the water content is about 5.5±0.1 wt %. In some embodiments, the water content is about 6±1 wt %. In some embodiments, the water content is about 6±0.5 wt %. In some embodiments, the water content is about 6.5±1 wt %. In some embodiments, the water content is about 6.5±0.5 wt %. In some embodiments, the water content is about 7±1 wt %. In some embodiments, the water content is about 7±0.5 wt %. In some embodiments, the water content is about 7.5±1 wt %. In some embodiments, the water content is about 7.5±0.5 wt %. In some embodiments, the water content is about 8±1 wt %. In some embodiments, the water content is about 8±0.5 wt %. In some embodiments, the water content is about 8.5±1 wt %. In some embodiments, the water content is about 8.5±0.5 wt %. In some embodiments, the water content is about 9±1 wt %. In some embodiments, the water content is about 9±0.5 wt %. In some embodiments, the water content is about 9.5±0.5 wt %. In some embodiments, the water content is about 9.5±0.25 wt %. In some embodiments, the water content is about 10±1 wt %. In some embodiments, the water content is about 10±0.5 wt %. In some embodiments, the water content is about 10±0.25 wt %. In some embodiments, the water content is about 11±1 wt %. In some embodiments, the water content is about 11±0.5 wt %. In some embodiments, the water content is about 11±0.25 wt %. In some embodiments, the water content is about 12±1 wt %. In some embodiments, the water content is about 12±0.5 wt %. In some embodiments, the water content is about 12±0.25 wt %. In some embodiments, the water content is about 13±1 wt %. In some embodiments, the water content is about 13±0.5 wt %. In some embodiments, the water content is about 13±0.25 wt %. In some embodiments, the water content is about 14±1 wt %. In some embodiments, the water content is about 14±0.5 wt %. In some embodiments, the water content is about 14±0.25 wt %. In some embodiments, the water content is about 15±1 wt %. In some embodiments, the water content is about 15±0.5 wt %. In some embodiments, the water content is about 15±0.25 wt %.

In some embodiments, the water content of Pattern A is about 9 wt % to about 9.4% wt %. In some embodiments, the water content of Pattern A is 9.2 wt %±0.1 wt %. In some embodiments, the water content of Pattern A is about 9.2 wt %. The water content can be determined by Karl Fischer analysis.

In some embodiments, the water content of Pattern B is about 7.2 wt % to about 7.6% wt %. In some embodiments, the water content of Pattern B is 7.4 wt %±0.1 wt %. In some embodiments, the water content of Pattern B is about 7.4 wt %. The water content can be determined by Karl Fischer analysis.

### Pharmaceutical Compositions and Formulations

Pharmaceutical compositions of any of the crystalline forms detailed herein, including Pattern A, Pattern B, or Pattern C, are embraced by this disclosure. Thus, the disclosure includes pharmaceutical compositions comprising a crystalline form disclosed herein (e.g., Pattern A, Pattern B, or Pattern C) and a pharmaceutically acceptable carrier or excipient. In one embodiment, the pharmaceutical composition is a composition for controlled release of any of the crystalline forms detailed herein.

In some embodiments, provided is a composition comprising Pattern A. In some embodiments, provided is a composition comprising Pattern A and a pharmaceutically acceptable carrier or excipient. In some embodiments, the composition is substantially free of amorphous or non-crystalline forms of (S)-4-((2-methoxyethyl)(4-(5,6,7,8-tetrahydro-1,8-naphthyridin-2-yl)butyl)amino)-2-(quinazolin-4-ylamino)butanoic acid or a salt thereof. In some embodiments of the composition comprising Pattern A, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Pattern A. In some embodiments of the composition comprising Pattern A, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of (S)-4-((2-methoxyethyl)(4-(5,6,7,8-tetrahydro-1,8-naphthyridin-2-yl)butyl)amino)-2-(quinazolin-4-ylamino)butanoic acid exists in Pattern A.

In some embodiments of the composition comprising Pattern A, the composition comprises up to about 10%, 9%, 8%, 7%, 6%, 5%, 4%, 3%, 2%, 1%, or 0.5% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 10% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 9% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 8% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 7% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 6% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 5% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 4% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 3% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 2% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 1% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A. In some embodiments of the composition comprising Pattern A, the composition comprises up to about 0.5% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern A.

In some embodiments, provided is a composition comprising Pattern B. In some embodiments, provided is a composition comprising Pattern B and a pharmaceutically acceptable carrier or excipient. In some embodiments, the composition is substantially free of amorphous or non-crystalline form of (S)-4-((2-methoxyethyl)(4-(5,6,7,8-tetrahydro-1,8-naphthyridin-2-yl)butyl)amino)-2-(quinazolin-4-ylamino)butanoic acid or a salt thereof. In some embodiments of the composition comprising Pattern B, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Pattern B. In some embodiments of the composition comprising Pattern B, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of (S)-4-((2-methoxyethyl)(4-(5,6,7,8-tetrahydro-1,8-naphthyridin-2-yl)butyl)amino)-2-(quinazolin-4-ylamino)butanoic acid exists in Pattern B.

In some embodiments of the composition comprising Pattern B, the composition comprises up to about 10%, 9%, 8%, 7%, 6%, 5%, 4%, 3%, 2%, 1%, or 0.5% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 10% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 9% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 8% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 7% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 6% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 5% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 4% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 3% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 2% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 1% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B. In some embodiments of the composition comprising Pattern B, the composition comprises up to about 0.5% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern B.

In some embodiments, provided is a composition comprising Pattern C. In some embodiments, provided is a composition comprising Pattern C and a pharmaceutically acceptable carrier or excipient. In some embodiments, the composition is substantially free of amorphous or non-crystalline form of (S)-4-((2-methoxyethyl)(4-(5,6,7,8-tetrahydro-1,8-naphthyridin-2-yl)butyl)amino)-2-(quinazolin-4-ylamino)butanoic acid or a salt thereof. In some embodiments of the composition comprising Pattern C, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Pattern C. In some embodiments of the composition comprising Pattern C, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of (S)-4-((2-methoxyethyl)(4-(5,6,7,8-tetrahydro-1,8-naphthyridin-2-yl)butyl)amino)-2-(quinazolin-4-ylamino)butanoic acid exists in Pattern C.

In some embodiments of the composition comprising Pattern C, the composition comprises up to about 10%, 9%, 8%, 7%, 6%, 5%, 4%, 3%, 2%, 1%, or 0.5% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 10% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 9% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 8% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 7% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 6% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 5% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 4% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 3% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 2% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 1% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C. In some embodiments of the composition comprising Pattern C, the composition comprises up to about 0.5% by weight of (S)-4-((2-methoxyethyl)(4-(5,6,7,8-tetrahydro-1,8-naphthyridin-2-yl)butyl)amino)-2-(quinazolin-4-ylamino)butanoic acid existing in a form other than Pattern C.

Crystalline forms or compositions disclosed herein may be formulated for any available delivery route, including an oral, mucosal (e.g., nasal, sublingual, vaginal, buccal or rectal), parenteral (e.g., intramuscular, subcutaneous or intravenous), topical or transdermal delivery form, or a form suitable for inhalation. A crystalline form or composition disclosed herein may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Crystalline forms disclosed herein can be used in the preparation of a formulation, such as a pharmaceutical formulation, by combining the crystalline form as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the crystalline form may also contain other substances that have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), the entire contents of which are incorporated herein by reference.

Crystalline forms disclosed herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Crystalline forms disclosed herein can be formulated in a tablet in any dosage form described, for example, a crystalline form can be formulated as a 10 mg tablet. Exemplary dosages and dosage forms for crystalline forms disclosed herein are disclosed in U.S. patent application Ser. No. 16/843,824, published as US Pat. App. Pub. No. 2020/0352942 and issued as U.S. Pat. No. 11,419,869; and U.S. patent application Ser. No. 17/733,488, published as US Pat. App. Pub. No. 2023/0028658; the entire contents of which are incorporated herein by reference.

In various embodiments, a dose, e.g., a unit dose, such as a unit dose for daily administration, can include the crystalline form in an amount of one of, or one of about: 1, 2.5, 5, 7.5, 10, 15, 20, 25, 30, 35, 40, 45, 50, 55, 60, 75, 80, 85, 90, 95, 100, 105, 110, 115, 120, 125, 130, 140, 150, 160, 170, 175, 180, 190, 200, 225, 240, 250, 275, 300, 320, 325, 350, 375, 400, 425, 450, 475, 480, 500, 525, 550, 560, 575, 600, 625, 640, 650, 675, 700, 720, 725, 750, 775, 800, 825, 850, 875, 880, 900, 925, 950, 960, 975, 1000, 1025, or 1040 milligrams. For example, a dose can include the crystalline form in an amount of, or of about, 10 mg. A dose can include the crystalline form in an amount of, or of about, 15 mg. A dose can include the crystalline form in an amount of, or of about, 20 mg. A dose can include the crystalline form in an amount of, or of about, 30 mg. A dose can include the crystalline form in an amount of, or of about, 40 mg. A dose can include the crystalline form in an amount of, or of about, 50 mg. A dose can include the crystalline form in an amount of, or of about, 75 mg. A dose can include the crystalline form in an amount of, or of about, 80 mg. A dose can include the crystalline form in an amount of, or of about, 100 mg. A dose can include the crystalline form in an amount of, or of about, 120 mg. A dose can include the crystalline form in an amount of, or of about, 160 mg. A dose can include the crystalline form in an amount of, or of about, 240 mg. A dose can include the crystalline form in an amount of, or of about, 320 mg. A dose can include the crystalline form in an amount of, or of about, 400 mg. A dose can include the crystalline form in an amount of, or of about, 480 mg. A dose can include the crystalline form in an amount of, or of about, 560 mg. A dose can include the crystalline form in an amount of, or of about, 640 mg. A dose can include the crystalline form in an amount of, or of about, 720 mg. A dose can include the crystalline form in an amount of, or of about, 800 mg. A dose can include the crystalline form in an amount of, or of about, 880 mg. A dose can include the crystalline form in an amount of, or of about, 960 mg. A dose can include the crystalline form in an amount of, or of about, 1040 mg.

In various embodiments, a dose, e.g., a unit dose, such as a unit dose for daily administration, can include the crystalline form in an amount comprising an amount of the crystalline form in mg of about one of about: 320, 400, 480, 560, 640, 720, 800, 880, 960, or 1040, or a range between any two of the preceding values.

In various embodiments, a dose, e.g., a unit dose, such as a unit dose for daily administration, can include the crystalline form in an amount comprising an amount of the crystalline form in mg of about one of about: 400, 480, 560, 640, 720, 800, 880, 960, or 1040.

In various embodiments, a dose, e.g., a unit dose, such as a unit dose for daily administration, can include the crystalline form in an amount comprising an amount of the crystalline form in mg of a range between about 320 and any one of about 400, 480, 560, 640, 720, 800, 880, 960, or 1040.

In various embodiments, a dose, e.g., a unit dose, such as a unit dose for daily administration, can include the crystalline form in an amount comprising an amount of the crystalline form in mg of about one of: 400, 480, 560, 640, 720, 800, 880, 960, or 1040, or a range between any two of the preceding values.

In some embodiments, the unit dose may include the crystalline form in a percentage range about any of the individual values in milligrams recited in the preceding paragraph, for example, any percentage range independently selected from one of, or one of about: +1%, +2%, +2.5%, +5%, +7.5%, +10%, +15%, +20%, +25%, +30%, +40%, or +50%. For example, the range may be, or be about, +1%. The range may be, or be about, +2%. The range may be, or be about, +2.5%. The range may be, or be about, +5%. The range may be, or be about, +7.5%. The range may be, or be about, +10%. The range may be, or be about, +15%. The range may be, or be about, +20%. The range may be, or be about, +25%. The range may be, or be about, +30%. The range may be, or be about, +40%. The range may be, or be about, +50%.

Further, for example, the unit dose may include the crystalline form in an amount of one of: 10 mg±1%; 10 mg±2%; 10 mg±2.5%; 10 mg±5%; 10 mg±7.5%; 10 mg±10%; 10 mg±15%; 10 mg±20%; 10 mg±25%; 10 mg±30%; 10 mg±40%; or 10 mg±50%. The unit dose may include the crystalline form in an amount of one of: 15 mg±1%; 15 mg±2%; 15 mg±2.5%; 15 mg±5%; 15 mg±7.5%; 15 mg±10%; 15 mg±15%; 15 mg±20%; 15 mg±25%; 15 mg±30%; 15 mg±40%; or 15 mg±50%. The unit dose may include the crystalline form in an amount of one of: 20 mg±1%; 20 mg±2%; 20 mg±2.5%; 20 mg±5%; 20 mg±7.5%; 20 mg±10%; 20 mg±15%; 20 mg±20%; 20 mg±25%; 20 mg±30%; 20 mg±40%; or 20 mg±50%. The unit dose may include the crystalline form in an amount of one of: 30 mg±1%; 30 mg±2%; 30 mg±2.5%; 30 mg±5%; 30 mg±7.5%; 30 mg±10%; 30 mg±15%; 30 mg±20%; 30 mg±25%; 30 mg±30%; 30 mg±40%; or 30 mg±50%. The unit dose may include the crystalline form in an amount of one of: 40 mg±1%; 40 mg±2%; 40 mg±2.5%; 40 mg±5%; 40 mg±7.5%; 40 mg±10%; 40 mg±15%; 40 mg±20%; 40 mg±25%; 40 mg±30%; 40 mg±40%; or 40 mg±50%. The unit dose may include the crystalline form in an amount of one of: 50 mg±1%; 50 mg±2%; 50 mg±2.5%; 50 mg±5%; 50 mg±7.5%; 50 mg±10%; 50 mg±15%; 50 mg±20%; 50 mg±25%; 50 mg±30%; 50 mg±40%; or 50 mg±50%. The unit dose may include the crystalline form in an amount of one of: 60 mg±1%; 60 mg±2%; 60 mg±2.5%; 60 mg±5%; 60 mg±7.5%; 60 mg±10%; 60 mg±15%; 60 mg±20%; 60 mg±25%; 60 mg±30%; 60 mg±40%; or 60 mg±50%. The unit dose may include the crystalline form in an amount of one of: 75 mg±1%; 75 mg±2%; 75 mg±2.5%; 75 mg±5%; 75 mg±7.5%; 75 mg±10%; 75 mg±15%; 75 mg±20%; 75 mg±25%; 75 mg±30%; 75 mg±40%; or 75 mg±50%. The unit dose may include the crystalline form in an amount of one of: 80 mg±1%; 80 mg±2%; 80 mg±2.5%; 80 mg±5%; 80 mg±7.5%; 80 mg±10%; 80 mg±15%; 80 mg±20%; 80 mg±25%; 80 mg±30%; 80 mg±40%; or 80 mg±50%. The unit dose may include the crystalline form in an amount of one of: 100 mg±1%; 100 mg±2%; 100 mg±2.5%; 100 mg±5%; 100 mg±7.5%; 100 mg±10%; 100 mg±15%; 100 mg±20%; 100 mg±25%; 100 mg±30%; 100 mg±40%; or 100 mg±50%. The unit dose may include the crystalline form in an amount of one of: 120 mg±1%; 120 mg±2%; 120 mg±2.5%; 120 mg±5%; 120 mg±7.5%; 120 mg±10%; 120 mg±15%; 120 mg±20%; 120 mg±25%; 120 mg±30%; 120 mg±40%; or 120 mg±50%. The unit dose may include the crystalline form in an amount of one of: 160 mg±1%; 160 mg±2%; 160 mg±2.5%; 160 mg±5%; 160 mg±7.5%; 160 mg±10%; 160 mg±15%; 160 mg±20%; 160 mg±25%; 160 mg±30%; 160 mg±40%; or 160 mg±50%. The unit dose may include the crystalline form in an amount of one of: 240 mg±1%; 240 mg±2%; 240 mg±2.5%; 240 mg±5%; 240 mg±7.5%; 240 mg±10%; 240 mg±15%; 240 mg±20%; 240 mg±25%; 240 mg±30%; 240 mg±40%; or 240 mg±50%. The unit dose may include the crystalline form in an amount of one of: 320 mg±1%; 320 mg±2%; 320 mg±2.5%; 320 mg±5%; 320 mg±7.5%; 320 mg±10%; 320 mg±15%; 320 mg±20%; 320 mg±25%; 320 mg±30%; 320 mg±40%; or 320 mg±50%. The unit dose may include the crystalline form in an amount of one of: 400 mg±1%; 400 mg±2%; 400 mg±2.5%; 400 mg±5%; 400 mg±7.5%; 400 mg±10%; 400 mg±15%; 400 mg±20%; 400 mg±25%; 400 mg±30%; 400 mg±40%; or 400 mg±50%. The unit dose may include the crystalline form in an amount of one of: 480 mg±1%; 480 mg±2%; 480 mg±2.5%; 480 mg±5%; 480 mg±7.5%; 480 mg±10%; 480 mg±15%; 480 mg±20%; 480 mg±25%; 480 mg±30%; 480 mg±40%; or 480 mg±50%. The unit dose may include the crystalline form in an amount of one of: 560 mg±1%; 560 mg±2%; 560 mg±2.5%; 560 mg±5%; 560 mg±7.5%; 560 mg±10%; 560 mg±15%; 560 mg±20%; 560 mg±25%; 560 mg±30%; 560 mg±40%; or 560 mg±50%. The unit dose may include the crystalline form in an amount of one of: 640 mg±1%; 640 mg±2%; 640 mg±2.5%; 640 mg±5%; 640 mg±7.5%; 640 mg±10%; 640 mg±15%; 640 mg±20%; 640 mg±25%; 640 mg±30%; 640 mg±40%; or 640 mg±50%. The unit dose may include the crystalline form in an amount of one of: 720 mg±1%; 720 mg±2%; 720 mg±2.5%; 720 mg±5%; 720 mg±7.5%; 720 mg±10%; 720 mg±15%; 720 mg±20%; 720 mg±25%; 720 mg±30%; 720 mg±40%; or 720 mg±50%. The unit dose may include the crystalline form in an amount of one of: 800 mg±1%; 800 mg±2%; 800 mg±2.5%; 800 mg±5%; 800 mg±7.5%; 800 mg±10%; 800 mg±15%; 800 mg±20%; 800 mg±25%; 800 mg±30%; 800 mg±40%; or 800 mg±50%. The unit dose may include the crystalline form in an amount of one of: 880 mg±1%; 880 mg±2%; 880 mg±2.5%; 880 mg±5%; 880 mg±7.5%; 880 mg±10%; 880 mg±15%; 880 mg±20%; 880 mg±25%; 880 mg±30%; 880 mg±40%; or 880 mg±50%. The unit dose may include the crystalline form in an amount of one of: 960 mg±1%; 960 mg±2%; 960 mg±2.5%; 960 mg±5%; 960 mg±7.5%; 960 mg±10%; 960 mg±15%; 960 mg±20%; 960 mg±25%; 960 mg±30%; 960 mg±40%; or 960 mg±50%. The unit dose may include the crystalline form in an amount of one of: 1040 mg±1%; 1040 mg±2%; 1040 mg±2.5%; 1040 mg±5%; 1040 mg±7.5%; 1040 mg±10%; 1040 mg±15%; 1040 mg±20%; 1040 mg±25%; 1040 mg±30%; 1040 mg±40%; or 1040 mg±50%.

A unit dose, such as a unit dose for daily administration, can comprise the crystalline form in an amount effective on administration to an individual to produce a Cₘₐₓ in plasma of the individual in ng/mL of at least about, or greater than about, one of: 700, 750, 800, 850, 900, 950, 1000, 1050, 1100, 1150, 1200, 1250, 1300, 1350, 1400, 1450, 1500, 1600, 1700, 1800, 1900, 2000, 2100, 2200, 2300, 2400, or 2500; or a range between any two of the preceding concentrations, such as 700-1500, 700-900, 800-1300, 750-950, 800-1000, 850-950, 850-1050, 900-1400, 900-1300, 900-1200, 900-1100, 950-1050, 950-1400, 950-1150, 1000-1400, 1000-1300, 1000-1200, 700-2500, 1000-2500, 1500-2500, 1500-2000, 1500-2500, 2000-2500, and the like. For example, Cₘₐₓ can be, or be about, about 700 ng/ml or greater. Cₘₐₓ can be, or be about, about 750 ng/ml or greater. Cₘₐₓ can be, or be about, about 800 ng/ml or greater. Cₘₐₓ can be, or be about, 850 ng/ml or greater. Cₘₐₓ can be, or be about, 900 ng/mL or greater. Cₘₐₓ can be, or be about, 950 ng/mL or greater. Cₘₐₓ can be, or be about, 1000 ng/ml or greater. Cₘₐₓ can be, or be about, 1050 ng/ml or greater. Cₘₐₓ can be, or be about, 1100 ng/ml or greater. Cₘₐₓ can be, or be about, 1200 ng/mL or greater. Cₘₐₓ can be, or be about, 1300 ng/ml or greater. Cₘₐₓ can be, or be about, 1400 ng/ml or greater. Cₘₐₓ can be, or be about, 1500 ng/mL or greater. Cₘₐₓ can be, or be about, 1600 ng/ml or greater. Cₘₐₓ can be, or be about, 1700 ng/mL or greater. Cₘₐₓ can be, or be about, 1800 ng/ml or greater. Cₘₐₓ can be, or be about, 1900 ng/mL or greater. Cₘₐₓ can be, or be about, 2000 ng/mL or greater. Cₘₐₓ can be, or be about, 2100 ng/mL or greater. Cₘₐₓ can be, or be about, 2200 ng/ml or greater. Cₘₐₓ can be, or be about, 2300 ng/ml or greater. Cₘₐₓ can be, or be about, 2400 ng/ml or greater. Cₘₐₓ can be, or be about, 2500 ng/mL or greater.

A unit dose, such as a unit dose for daily administration, can comprise the crystalline form in an amount effective on administration to an individual to produce a Cₘₐₓ in plasma of the individual in ng/ml of at least about one of 700, 750, 800, 850, 900, 950, 1000, 1050, 1100, 1150, 1200, 1250, 1300, 1350, 1400, 1450, or 1500, or a range between any two of the preceding concentrations.

A unit dose, such as a unit dose for daily administration, can comprise the crystalline form in an amount effective on administration to an individual to produce a Cₘₐₓ in plasma of the individual in ng/mL in a range between of at least about any one of 700, 750, 800, 850, 900, 950, 1000, 1050, 1100, 1150, 1200, 1250, 1300, 1350, 1400, or 1450 as a lower limit and 1500 as an upper limit.

A unit dose, such as a unit dose for daily administration, can comprise the crystalline form in an amount effective on administration to an individual to produce a Cₘₐₓ in plasma of the individual in ng/ml of at least about one of: 1500, 1600, 1700, 1800, 1900, 2000, 2100, 2200, 2300, 2400, or 2500, or a range between any two of the preceding concentrations.

A unit dose, such as a unit dose for daily administration, can comprise the crystalline form in an amount effective on administration to an individual to produce a Cₘₐₓ in plasma of the individual in ng/ml of at least about one of: 1600, 1700, 1800, 1900, 2000, 2100, 2200, 2300, 2400, or 2500, or a range between any two of the preceding concentrations.

A unit dose, such as a unit dose for daily administration, can comprise the crystalline form in an amount effective on administration to an individual to produce a Cₘₐₓ in plasma of the individual in ng/mL in a range between at least 1500 and any one of 1600, 1700, 1800, 1900, 2000, 2100, 2200, 2300, 2400, or 2500.

A unit dose, such as a unit dose for daily administration, can comprise the crystalline form in an amount effective on administration to an individual to produce a Cₘₐₓ in ng/ml in plasma of the individual, the Cₘₐₓ corresponding to a plasma-adjusted concentration effective to inhibit a percentage of αᵥβ₆ or αᵥβ₁ in the individual of at least one of, or at least about one of: 50, 55, 60, 65, 70, 75, 80, 85, 90, 95, 97, 98, 99, or 100, or a range between any two of the preceding percentages, for example, 50-100, 60-90, 70-90, 75-95, 90-95, 90-98, 90-99, and the like. In some embodiments, the crystalline form may be a dual αᵥβ₆ and αᵥβ₁ inhibitor, and the Cₘₐₓ can correspond to a plasma-adjusted concentration effective to inhibit a percentage of each of αᵥβ₆ and αᵥβ₁ in the individual, each percentage independently selected from the preceding percentages, or a range between any two of the preceding percentages. For example, the plasma-adjusted concentration can be effective to inhibit αᵥβ₆ by at least about 50%. The plasma-adjusted concentration can be effective to inhibit αᵥβ₆ by at least about 60%. The plasma-adjusted concentration can be effective to inhibit αᵥβ₆ by at least about 70%. The plasma-adjusted concentration can be effective to inhibit αᵥβ₆ by at least about 80%. The plasma-adjusted concentration can be effective to inhibit αᵥβ₆ by at least about 90%. The plasma-adjusted concentration can be effective to inhibit αᵥβ₆ by at least about 95%. The plasma-adjusted concentration can be effective to inhibit αᵥβ₆ by at least about 97%. The plasma-adjusted concentration can be effective to inhibit αᵥβ₆ by at least about 98%. The plasma-adjusted concentration can be effective to inhibit αᵥβ₆ by at least about 99%. The plasma-adjusted concentration can be effective to inhibit αᵥβ₆ by about 100%. Further, for example, the plasma-adjusted concentration can be effective to inhibit αᵥβ₁ by at least about 50%. The plasma-adjusted concentration can be effective to inhibit αᵥβ₁ by at least about 60%. The plasma-adjusted concentration can be effective to inhibit αᵥβ₁ by at least about 70%. The plasma-adjusted concentration can be effective to inhibit αᵥβ₁ by at least about 80%. The plasma-adjusted concentration can be effective to inhibit αᵥβ₁ by at least about 90%. The plasma-adjusted concentration can be effective to inhibit αᵥβ₁ by at least about 95%. The plasma-adjusted concentration can be effective to inhibit αᵥβ₁ by at least about 97%. The plasma-adjusted concentration can be effective to inhibit αᵥβ₁ by at least about 98%. The plasma-adjusted concentration can be effective to inhibit αᵥβ₁ by at least about 99%. The plasma-adjusted concentration can be effective to inhibit αᵥβ₁ by about 100%. The recitation “percentage of each of αᵥβ₆ and/or αᵥβ₁ in the individual, each percentage independently selected” means, in the alternative, a single αᵥβ₆ inhibitor and corresponding percentage, a single αᵥβ₁ inhibitor and corresponding percentage, or a dual αᵥβ₆/αᵥβ₆ inhibitor and corresponding independently selected percentages. The recitation “percentage of each of αᵥβ₆ and/or αᵥβ₁ in the individual, each percentage independently selected” means, in the alternative, a single αᵥβ₆ inhibitor and corresponding percentage, a single αᵥβ₁ inhibitor and corresponding percentage, or a dual αᵥβ₆/αᵥβ₆ inhibitor and corresponding independently selected percentages.

The dosage form for daily administration can be administered to an individual in need thereof once daily. That is, the total amount of a crystalline form that is to be administered each day can be administered all together at one time daily. Alternatively, if it is desirable that the total amount of a crystalline form is to be administered in two or more portions daily, the dosage form containing the appropriate amount of crystalline form can be administered two times or more daily, such as twice a day, three times a day, or four times a day.

Compositions comprising a crystalline form disclosed herein are also described. In some embodiments, the composition is for use as a human or veterinary medicament. In some embodiments, the composition is for use in a method described herein. In some embodiments, the composition is for use in the treatment of a disease or disorder described herein.

### Methods of Making Crystalline Forms

PATTERN A. In one embodiment, provided herein is a method of making(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A, comprising:
mixing(S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid in a solvent;
heating the mixture to dissolve at least a portion of the (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid;
cooling the mixture;
stirring the mixture;
optionally adding additional solvent to the mixture; and
collecting a solid precipitate from the mixture. The solid precipitate is designated as crude Pattern A material. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid are combined in about a 1:2 molar ratio of (S)-4-((2-methoxyethyl)(4-(5,6,7,8-tetrahydro-1,8-naphthyridin-2-yl)butyl)amino)-2-(quinazolin-4-ylamino)butanoic acid to L-tartaric acid. In some embodiments, the mixture comprises a solution prior to heating. In some embodiments, the mixture is heated to form a solution. In some embodiments, the solvent comprises an alcohol. In some embodiments, the solvent comprises methanol or ethanol. In some embodiments, the solvent comprises methanol or ethanol, optionally with 5% water by volume. In some embodiments, the solvent is methanol with 5% water by volume. In some embodiments, the mixture is heated to about 40° C. to about 60° C., to about 45° C. to about 55° C., or to about 50° C. In some embodiments, the heated mixture is stirred for about 1 hour to about 4 hours, for about 90 minutes to about 3 hours, or for about 2 hours. In some embodiments, the mixture is cooled to about 20° C. to about 30° C., or about 25° C., or about room temperature. In some embodiments, the cooled mixture is stirred for about 1 day to about 4 days, for about 1 day to about 3 days, for about 1 day to about 2 days, or for about 2 days. In some embodiments, the additional solvent comprises an alcohol. In some embodiments, the additional solvent comprises an alcohol, such as methanol or ethanol optionally with 5% water by volume, such as methanol with 5% water by volume. In some embodiments, the solid precipitate is collected by centrifugation.

In further embodiments, solid precipitate from the procedure above is collected for use as seeds, and the procedure is repeated, with addition of the seeds after heating the mixture and before cooling the mixture.

In a further embodiment, the crude Pattern A material from the procedure above is dried under vacuum to form dried Pattern A material. The precipitate can be dried for about 30 minutes to about 5 hours, such as about 1 hour to about 3 hours, or about 2 hours. The precipitate can be dried at about room temperature or about 25° C. The precipitate can be dried at about 50° C. The precipitate can be dried for a first period at about room temperature or about 25° C., followed by drying for a second period at about 40° C. to about 60° C., about 45° C. to about 55° C., or about 50° C. The first period can be from about 10 minutes to about 2 hours, from about 15 minutes to about 1 hour, or for about 30 minutes. The second period can be from about 30 minutes to about 4 hours, about 1 hour to 2 hours, or about 90 minutes.

In some embodiments, enhanced crystallinity Pattern A material is prepared by temperature cycling crude or dried Pattern A material in an alcohol/water mixture, such as a mixture of isopropanol (IPA) and water. In some embodiments, crude or dried Pattern A material is suspended and equilibrated in an isopropanol/water mixture (which can be about 80:20 to about 95:5 IPA/H₂O v:v, about 85:15 to about 93:7 IPA/H₂O v:v, or about 89:11 IPA/H₂O v:v). In some embodiments, the suspension is cycled between about 5° C. to about 50° C. at a heating/cooling rate of about 0.1° C./min. In some embodiments, temperature cycling is conducted for about 5 days to about 30 days, such as for about 10 days to about 25 days, or for about 19 days or 20 days. In some embodiments, additional IPA/H₂O is optionally added during the equilibration. In some embodiments, after temperature cycling is completed, the resulting enhanced crystalline Pattern A material is dried under vacuum, at room temperature or at elevated temperature (such as at about 50° C.).

PATTERN B: In one embodiment, provided herein is a method of making(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B, comprising:
dissolving(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A in a volume of water to make an aqueous solution;
filtering the aqueous solution;
adding an alcohol to the aqueous solution to make a first alcohol/water solution;
stirring the first alcohol/water solution; and
collecting a solid precipitate from the first alcohol/water solution. In one embodiment, the alcohol is isopropanol (IPA). In one embodiment, a same volume of alcohol is added as the volume of water. In one embodiment, the method is carried out between about 20° C. to about 30° C., between about 22° C. to about 27° C., or at about room temperature. In one embodiment, an optional first additional volume of alcohol, such as isopropanol, is added to the first alcohol/water solution to form a second alcohol/water solution. The optional first additional volume of alcohol can be added in a volume about equal to the volume of the first alcohol/water solution. In one embodiment, an optional second additional volume of alcohol, such as isopropanol, is added to the second alcohol/water solution to form a third alcohol/water solution. The optional second additional volume of alcohol can be added in a volume about equal to the volume of the optional first additional volume of alcohol. The first alcohol/water solution can be stirred for about 1 day to about 5 days, about 1 day to about 3 days, or about 2 days. The second alcohol/water solution can be stirred for about 1 day to about 5 days, about 1 day to about 3 days, or about 2 days. The third alcohol/water solution can be stirred for about 1 day to about 5 days, about 1 day to about 3 days, or about 2 days. In a further embodiment, solid precipitate from the procedure above is collected for use as seeds, and the procedure is repeated, with addition of the seeds to either the first alcohol/water solution or the second alcohol water solution.

PATTERN C: In one embodiment, provided herein is a first method of making(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C, comprising:
dissolving(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A in an alcohol/water solution, such as an isopropanol (IPA)/water solution at a temperature above room temperature to form a warm solution;
filtering the warm solution;
stirring the warm solution;
cooling the warm solution to form a cooled solution;
stirring the cooled solution; and
collecting a solid precipitate from the cooled solution. The temperature above room temperature can be about 40° C. to about 60° C., about 45° C. to about 55° C., or about 50° C. The alcohol/water solution can be about 6:4 to about 8:2 alcohol to water (v:v), such as about 7:3 alcohol to water v:v. The alcohol can be isopropanol. The warm solution can be stirred at the temperature above room temperature for about 15 minutes to about 60 minutes, such as about 30 minutes. The warm solution can be cooled to about 0° C. to about 10° C., or about 5° C. The rate of cooling of the warm solution can be about 0.05° C./min to about 0.15° C./min, such as 0.1° C./min. The cooled solution can be stirred for about 1 day to about 2 days, such as about 2 days. The solid precipitate can be collected by centrifugation.

In one embodiment, provided herein is a second method of making(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C, comprising:
dissolving(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A in an alcohol/water solution, such as an isopropanol (IPA)/water solution at a temperature above room temperature to form a warm solution;
filtering the warm solution;
optionally adding Pattern C seed crystals from the first method of making(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C to the warm solution;
cooling the warm solution to form a cooled solution;
stirring the cooled solution;
collecting a solid precipitate from the cooled solution; and
incubating the solid precipitate under controlled temperature and humidity conditions for a period of time. The temperature above room temperature can be about 40° C. to about 60° C., about 45° C. to about 55° C., or about 50° C. The alcohol/water solution can be about 6:4 to about 8:2 alcohol to water (v:v), such as about 7:3 alcohol to water v:v. The alcohol can be isopropanol. The warm solution can be cooled to about 0° C. to about 10° C., or about 5° C. The rate of cooling of the warm solution can be about 0.05° C./min to about 0.15° C./min, such as 0.1° C./min. The cooled solution can be stirred for about 1 day to about 2 days, such as about 2 days. The solid precipitate can be collected by centrifugation. The solid precipitate can be incubated at a relative humidity (RH) of about 10% to about 20% RH at about 17° C. to about 30° C., between about 21° C. to about 26° C., or at about room temperature. The solid precipitate can be incubated for about 12 hours to about 2 days, such as about 24 hours.

### Methods of Use

Crystalline forms and compositions disclosed herein may be used in methods of administration and treatment as provided herein. The crystalline forms and compositions may also be used in in vitro methods, such as in vitro methods of administering a compound or composition to cells for screening purposes and/or for conducting quality control assays.

In some embodiments, provided is a method of treating a disease in an individual in need thereof comprising administering to the individual an effective amount of a crystalline form disclosed herein (e.g., Pattern A, Pattern B, or Pattern C). In some embodiments, provided is a method of treating a disease in an individual in need thereof comprising administering to the individual a therapeutically effective amount of a crystalline form disclosed herein (e.g., Pattern A, Pattern B, or Pattern C). In some embodiments, the individual is a human. The individual, such as human, may be in need of treatment, such as a human. In some embodiments, the disease is selected from the group consisting of progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, and Crohn's Disease.

In some embodiments, provided is a method of treating a fibrotic disease in an individual in need thereof comprising administering to the individual an effective amount of a crystalline form disclosed herein (e.g., Pattern A, Pattern B, or Pattern C). In some embodiments, provided is a method of treating a fibrotic disease in an individual in need thereof comprising administering to the individual a therapeutically effective amount of a crystalline form disclosed herein (e.g., Pattern A, Pattern B, or Pattern C). In some embodiments, the individual is a human. The individual, such as human, may be in need of treatment, such as a human who has or is suspected of having a fibrotic disease.

In some embodiments, provided is a method of delaying the onset and/or development of a fibrotic disease in an individual (such as a human) who is at risk for developing a fibrotic disease, comprising administering to the individual an effective amount of a crystalline form disclosed herein (e.g., Pattern A, Pattern B, or Pattern C). In some embodiments, provided is a method of delaying the onset and/or development of a fibrotic disease in an individual (such as a human) who is at risk for developing a fibrotic disease, comprising administering to the individual a therapeutically effective amount of a crystalline form disclosed herein (e.g., Pattern A, Pattern B, or Pattern C). It is appreciated that delayed development may encompass prevention in the event the individual does not develop the fibrotic disease. An individual at risk of developing a fibrotic disease in one aspect has or is suspected of having one or more risk factors for developing a fibrotic disease. Risk factors for fibrotic disease may include an individual's age (e.g., middle-age or older adults), the presence of inflammation, having one or more genetic component associated with development of a fibrotic disease, medical history such as treatment with a drug or procedure believed to be associated with an enhanced susceptibility to fibrosis (e.g., radiology) or a medical condition believed to be associated with fibrosis, a history of smoking, the presence of occupational and/or environmental factors such as exposure to pollutants associated with development of a fibrotic disease. In some embodiments, the individual at risk for developing a fibrotic disease is an individual who has or is suspected of having NAFLD, NASH, CKD, scleroderma, Crohn's Disease, NSIP, PSC, PBC, or is an individual who has had or is suspected of having had a myocardial infarction.

In some embodiments, the fibrotic disease is fibrosis of a tissue such as the lung (pulmonary fibrosis), the liver, the skin, the heart (cardiac fibrosis), the kidney (renal fibrosis), or the gastrointestinal tract (gastrointestinal fibrosis).

In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC).

In some embodiments, the fibrotic disease is a pulmonary fibrosis, e.g., idiopathic pulmonary fibrosis (IPF). In some embodiments, the pulmonary fibrosis is, e.g., interstitial lung disease, radiation-induced pulmonary fibrosis, or systemic sclerosis associated interstitial lung disease. In some embodiments, the pulmonary fibrosis is IPF, progressive pulmonary fibrosis, rheumatoid arthritis-associated interstitial lung disease, interstitial lung disease, radiation-induced pulmonary fibrosis, or systemic sclerosis associated interstitial lung disease.

In some embodiments, the fibrotic disease is a primary sclerosing cholangitis, or biliary fibrosis. In some embodiments, the fibrotic disease is primary biliary cholangitis (also known as primary biliary cirrhosis) or biliary atresia.

In some embodiments, the fibrotic disease is fibrotic nonspecific interstitial pneumonia (NSIP).

In some embodiments, the fibrotic disease is a liver fibrosis, e.g., infectious liver fibrosis (from pathogens such as HCV, HBV or parasites such as schistosomiasis), NASH, alcoholic steatosis induced liver fibrosis, and cirrhosis. In some embodiments, the liver fibrosis is nonalcoholic fatty liver disease (NAFLD). In some embodiments, the liver fibrosis is NASH.

In some embodiments, the fibrotic disease is biliary tract fibrosis.

In some embodiments, the fibrotic disease is renal fibrosis, e.g., diabetic nephrosclerosis, hypertensive nephrosclerosis, focal segmental glomerulosclerosis (“FSGS”), and acute kidney injury from contrast induced nephropathy. In several embodiments, the fibrotic disease is diabetic nephropathy, diabetic kidney disease, or chronic kidney disease.

In some embodiments, the fibrotic disease is characterized by one or more of glomerulonephritis, end-stage kidney disease, hearing loss, changes to the lens of the eye, hematuria, or proteinuria. In some embodiments, the fibrotic disease is Alport syndrome.

In some embodiments, the fibrotic disease is systemic and local sclerosis or scleroderma, keloids and hypertrophic scars, or post-surgical adhesions. In some embodiments, the fibrotic disease is scleroderma or systemic sclerosis.

In some embodiments, the fibrotic disease is atherosclerosis or restenosis.

In some embodiments, the fibrotic disease is a gastrointestinal fibrosis, e.g., Crohn's disease.

In some embodiments, the fibrotic disease is cardiac fibrosis, e.g., post myocardial infarction induced fibrosis and inherited cardiomyopathy.

In some embodiments, methods may include modulating the activity of at least one integrin in an individual in need thereof. For example, the method may include modulating the activity of αᵥβ₆. The method may include modulating the activity of αᵥβ₁. The method may include modulating the activity of αᵥβ₁ and αᵥβ₆. Modulating the activity of the at least one integrin may include, e.g., inhibiting the at least one integrin. The method may include administering to the individual an amount of a crystalline form effective to modulate the activity of the at least one integrin in the individual, e.g., at least one of αᵥβ₁ and αᵥβ₆. The individual in need of modulating the activity of at least one integrin may have any of the fibrotic disease or conditions described herein. For example, the fibrotic disease or condition may include idiopathic pulmonary fibrosis, interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis, primary biliary cholangitis (also known as primary biliary cirrhosis), biliary atresia, systemic sclerosis associated interstitial lung disease, scleroderma (also known as systemic sclerosis), diabetic nephropathy, diabetic kidney disease, focal segmental glomerulosclerosis, chronic kidney disease, or Crohn's Disease. The method may include administering to the individual an amount of a crystalline form effective to modulate the activity of the at least one integrin in the individual, e.g., at least one of αᵥβ₁ and αᵥβ₆, the individual being in need of treatment for NASH. The method may include administering to the individual an amount of a crystalline form effective to modulate the activity of the at least one integrin in the individual, e.g., at least one of αᵥβ₁ and αᵥβ₆, the individual being in need of treatment for IPF.

The fibrotic disease may be mediated primarily by αᵥβ₆, for example, the fibrotic disease may include idiopathic pulmonary fibrosis or renal fibrosis. Accordingly, the method may include modulating the activity of αᵥβ₆ to treat conditions primarily mediated by αᵥβ₆ such as IPF. The fibrotic disease may be mediated primarily by αᵥβ₁, for example, the fibrotic disease may include NASH. Accordingly, the method may include modulating the activity of αᵥβ₁ to treat conditions primarily mediated by αᵥβ₁, e.g., NASH. The fibrotic disease may be mediated by αᵥβ₁ and αᵥβ₆, for example, the fibrotic disease may include PSC or biliary atresia. Accordingly, the method may include modulating the activity of αᵥβ₁ and αᵥβ₆ to treat conditions mediated by both αᵥβ₁ and αᵥβ₆.

The crystalline form may be a modulator, e.g., an inhibitor, of αᵥβ₁. The crystalline form may be a modulator, e.g., an inhibitor, of αᵥβ₆. The crystalline form may be a dual modulator, such as a dual inhibitor, e.g., dual selective inhibitor, of αᵥβ₁ and αᵥβ₆.

Modulating or inhibiting the activity of one or both of αᵥβ₁ integrin and αᵥβ₆ integrin, thereby treating an individual with a fibrotic disease, indicates that αᵥβ₁ integrin, αᵥβ₆ integrin, or αᵥβ₁ integrin and αᵥβ₆ integrin are modulated or inhibited to a degree sufficient to treat the fibrotic disease in the individual.

In some embodiments, provided is a crystalline form (e.g., Pattern A, Pattern B, or Pattern C) for use in the treatment of a fibrotic disease. Also provided is use of a crystalline form (e.g., Pattern A, Pattern B, or Pattern C) in the manufacture of a medicament for the treatment of a fibrotic disease.

In some embodiments, provided is a method of inhibiting αᵥβ₆ integrin in an individual comprising administering a crystalline form disclosed herein (e.g., Pattern A, Pattern B, or Pattern C).

Also provided is a method of inhibiting TGFβ activation in an individual in need thereof, comprising administering to the individual a crystalline form disclosed herein (e.g., Pattern A, Pattern B, or Pattern C).

Also provided is a method of inhibiting αᵥβ₁ integrin and/or αᵥβ₆ integrin in an individual in need thereof, comprising administering to the individual a crystalline form disclosed herein (e.g., Pattern A, Pattern B, or Pattern C). In all such embodiments, in one aspect the method of inhibition is for an individual in need thereof, such as an individual who has or is suspected of having a fibrotic disease, and wherein the method comprises administering to the individual a crystalline form disclosed herein (e.g., Pattern A, Pattern B, or Pattern C).

In any of the described methods, in one aspect the individual is a human, such as a human in need of the method. The individual may be a human who has been diagnosed with or is suspected of having a fibrotic disease. The individual may be a human who does not have detectable disease but who has one or more risk factors for developing a fibrotic disease.

In some embodiments, disclosed herein is a method of treating a disease in an individual in need thereof comprising administering to the individual any of the crystalline forms of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or solvates thereof, or pharmaceutical compositions thereof, wherein the disease is selected from the group consisting of fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, and Crohn's Disease.

In some embodiments, disclosed herein is a method of treating idiopathic pulmonary fibrosis in an individual in need thereof comprising administering to the individual any of the crystalline forms of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or solvates thereof, or pharmaceutical compositions thereof.

In some embodiments, disclosed herein is a method of treating progressive pulmonary fibrosis in an individual in need thereof comprising administering to the individual any of the crystalline forms of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or solvates thereof, or pharmaceutical compositions thereof.

In some embodiments, disclosed herein is a method of treating primary sclerosing cholangitis in an individual in need thereof comprising administering to the individual any of the crystalline forms of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or solvates thereof, or pharmaceutical compositions thereof.

In some embodiments, disclosed herein is a method of treating a disease in an individual in need thereof comprising administering to the individual a cocrystal of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising a cocrystal of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient, wherein the disease is selected from the group consisting of fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, and Crohn's Disease.

In some embodiments, disclosed herein is a method of treating idiopathic pulmonary fibrosis in an individual in need thereof comprising administering to the individual a cocrystal of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising a cocrystal of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating progressive pulmonary fibrosis in an individual in need thereof comprising administering to the individual a cocrystal of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising a cocrystal of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating primary sclerosing cholangitis in an individual in need thereof comprising administering to the individual a cocrystal of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising a cocrystal of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating a disease in an individual in need thereof comprising administering to the individual the Pattern A crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern A crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient, wherein the disease is selected from the group consisting of fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, and Crohn's Disease.

In some embodiments, disclosed herein is a method of treating idiopathic pulmonary fibrosis in an individual in need thereof comprising administering to the individual the Pattern A crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern A crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating progressive pulmonary fibrosis in an individual in need thereof comprising administering to the individual the Pattern A crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern A crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating primary sclerosing cholangitis in an individual in need thereof comprising administering to the individual the Pattern A crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern A crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating a disease in an individual in need thereof comprising administering to the individual the Pattern B crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern B crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient, wherein the disease is selected from the group consisting of fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, and Crohn's Disease.

In some embodiments, disclosed herein is a method of treating idiopathic pulmonary fibrosis in an individual in need thereof comprising administering to the individual the Pattern B crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern B crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating progressive pulmonary fibrosis in an individual in need thereof comprising administering to the individual the Pattern B crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern B crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating primary sclerosing cholangitis in an individual in need thereof comprising administering to the individual the Pattern B crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern B crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating a disease in an individual in need thereof comprising administering to the individual the Pattern C crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern C crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient, wherein the disease is selected from the group consisting of fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, and Crohn's Disease.

In some embodiments, disclosed herein is a method of treating idiopathic pulmonary fibrosis in an individual in need thereof comprising administering to the individual the Pattern C crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern C crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating progressive pulmonary fibrosis in an individual in need thereof comprising administering to the individual the Pattern C crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern C crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, disclosed herein is a method of treating primary sclerosing cholangitis in an individual in need thereof comprising administering to the individual the Pattern C crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, or a pharmaceutical composition comprising Pattern C crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or a solvate thereof, and a pharmaceutically acceptable carrier or excipient.

In some embodiments, the disease comprises fibrosis. In some embodiments, the fibrosis is pulmonary fibrosis, liver fibrosis, skin fibrosis, cardiac fibrosis, kidney fibrosis, gastrointestinal fibrosis, primary sclerosing cholangitis, or biliary fibrosis. In some embodiments, the fibrotic disease is liver fibrosis, cardiac fibrosis, primary sclerosing cholangitis, or biliary fibrosis. In some embodiments, the fibrotic disease is idiopathic pulmonary fibrosis. In some embodiments, the fibrotic disease is progressive pulmonary fibrosis. In some embodiments, the fibrotic disease is primary sclerosing cholangitis.

In some embodiments, the disease is progressive pulmonary fibrosis, pulmonary fibrosis associated with rheumatoid arthritis, idiopathic pulmonary fibrosis (IPF), radiation-induced pulmonary fibrosis, alcoholic liver disease induced fibrosis, or primary sclerosing cholangitis (PSC). In some embodiments, the disease is interstitial lung disease. In some embodiments, the disease is rheumatoid arthritis associated interstitial lung disease.

In some embodiments, disclosed herein is use of any of the crystalline forms of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or solvates thereof, or pharmaceutical compositions thereof in the manufacture of a medicament for the treatment of a fibrotic disease.

In some embodiments, disclosed herein is use of any of the crystalline forms of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or solvates thereof, or pharmaceutical compositions thereof in the manufacture of a medicament for the treatment of a disease mediated by at least one of αᵥβ₁ integrin and αᵥβ₆ integrin.

In some embodiments, disclosed herein is any of the crystalline forms of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or solvates thereof, or pharmaceutical compositions thereof for use in treating a fibrotic disease.

In some embodiments, disclosed herein is any of the crystalline forms of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or solvates thereof, or pharmaceutical compositions thereof for use in treating a disease mediated by at least one of αᵥβ₁ integrin and αᵥβ₆ integrin.

In some embodiments, disclosed herein is a method of modulating the activity of at least one integrin in an individual in need thereof, comprising administering to the individual an amount of any of the crystalline forms of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid disclosed herein, or solvates thereof, or pharmaceutical compositions thereof effective to modulate the activity of the at least one integrin in the individual, the at least one integrin including at least one of αᵥβ₁ integrin and αᵥβ₆ integrin. In some embodiments, the individual has or is at risk of a fibrotic disease selected from the group consisting of idiopathic pulmonary fibrosis, progressive pulmonary fibrosis, interstitial lung disease, radiation-induced pulmonary fibrosis, nonalcoholic fatty liver disease, nonalcoholic steatohepatitis, alcoholic liver disease induced fibrosis, Alport syndrome, primary sclerosing cholangitis, primary biliary cholangitis, biliary atresia, systemic sclerosis associated interstitial lung disease, scleroderma, diabetic nephropathy, diabetic kidney disease, focal segmental glomerulosclerosis, chronic kidney disease, and Crohn's Disease. In some embodiments, the individual is in need of treatment for idiopathic pulmonary fibrosis. In some embodiments, the individual is in need of treatment for progressive pulmonary fibrosis. In some embodiments, the individual is in need of treatment for primary sclerosing cholangitis.

In any of the described methods, the methods may employ any of the crystalline forms disclosed herein in any of the forms and doses detailed herein.

### Kits

In another aspect, provided is a kit comprising one or more crystalline forms disclosed herein (e.g., Pattern A, Pattern B, and/or Pattern C) or a pharmaceutical composition comprising a crystalline form as described herein. The kits may employ any of the crystalline forms disclosed herein. The kits may be used for any one or more of the uses described herein, and, accordingly, may contain instructions for use in the treatment of a fibrotic disease.

Kits generally comprise suitable packaging. The kits may comprise one or more containers comprising any compound described herein. Each component (if there is more than one component) can be packaged in separate containers or some components can be combined in one container where cross-reactivity and shelf-life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., an effective amount, such as a therapeutically effective amount) and/or a second pharmaceutically active compound useful for a disease detailed herein (e.g., fibrosis) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the crystalline forms and instructions for use and be packaged in quantities sufficient for storage and use in pharmacies (e.g., hospital pharmacies and compounding pharmacies). In some embodiments, kits comprise unit dosage forms, such as those disclosed herein.

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### Enumerated Embodiments

The following enumerated embodiments are representative of some aspects of the disclosure.

Enumerated embodiment 1A. A crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid, or a solvate thereof.

Enumerated embodiment 2A. The crystalline form of enumerated embodiment 1A, wherein the crystalline form is a cocrystal.

Enumerated embodiment 3A. The cocrystal of enumerated embodiment 2A, wherein the molar ratio of (S)-4-((2-methoxyethyl)(4-(5,6,7,8-tetrahydro-1,8-naphthyridin-2-yl)butyl)amino)-2-(quinazolin-4-ylamino)butanoic acid to L-tartaric acid is about 1:2.

Enumerated embodiment 4A. The crystalline form of any one of enumerated embodiments 1A-3A, wherein the crystalline form is a solvate and the solvate is a hydrate.

Enumerated embodiment 5A. The crystalline form of any one of enumerated embodiments 1A-4A, wherein the crystalline form is characterized by an XRPD pattern substantially as shown in FIG. 1.

Enumerated embodiment 6A. The crystalline form of any one of enumerated embodiments 1A-5A, wherein the crystalline form is characterized by an XRPD pattern comprising:
a. peaks at angles 2-theta of 8.11±0.2 degrees and 10.93±0.2 degrees;
b. peaks at angles 2-theta of 8.11±0.2 degrees, 10.93±0.2 degrees, and 14.91±0.2 degrees; or
c. peaks at angles 2-theta of 8.11±0.2 degrees, 10.93±0.2 degrees, 14.91±0.2 degrees, and 22.46±0.2 degrees.

Enumerated embodiment 7A. The crystalline form of any one of enumerated embodiments 1A-6A, wherein the crystalline form is characterized by an XRPD pattern comprising:
a. peaks at angles 2-theta of 6.94±0.2 degrees and 22.46±0.2 degrees;
b. peaks at angles 2-theta of 6.94±0.2 degrees, 10.93±0.2 degrees, and 22.46±0.2 degrees; or
c. peaks at angles 2-theta of 6.94±0.2 degrees, 8.47±0.2 degrees, 10.93±0.2 degrees, 22.46±0.2 degrees, and 24.78±0.2 degrees.

Enumerated embodiment 8A. The crystalline form of any one of enumerated embodiments 1A-7A, wherein the crystalline form is characterized by an XRPD pattern comprising:
a. peaks at angles 2-theta of 6.94±0.2 degrees and 10.93±0.2 degrees;
b. peaks at angles 2-theta of 6.94±0.2 degrees, 8.47±0.2 degrees, and 10.93±0.2 degrees; or
c. peaks at angles 2-theta of 6.94±0.2 degrees, 8.47±0.2 degrees, 10.93±0.2 degrees, 14.45±0.2 degrees, and 14.91±0.2 degrees.

Enumerated embodiment 9A. The crystalline form of any one of enumerated embodiments 1A-8A, wherein the crystalline form is characterized by a DSC graph substantially as shown in FIG. 2.

Enumerated embodiment 10A. The crystalline form of any one of enumerated embodiments 1A-9A, wherein the crystalline form is characterized by endotherm peaks at about 74.7° C. and about 139.9° C., as determined by DSC.

Enumerated embodiment 11A. The crystalline form of any one of enumerated embodiments 1A-10A, wherein the crystalline form is characterized by a TGA graph substantially as shown in FIG. 3.

Enumerated embodiment 12A. The crystalline form of any one of enumerated embodiments 1A-11A, wherein the crystalline form is characterized by a weight loss of about 8% after heating from about 34° C. to about 120° C., as determined by TGA.

Enumerated embodiment 13A. The crystalline form of any of enumerated embodiments 1A-12A, wherein the crystalline form has a water content of about 9.2% by weight.

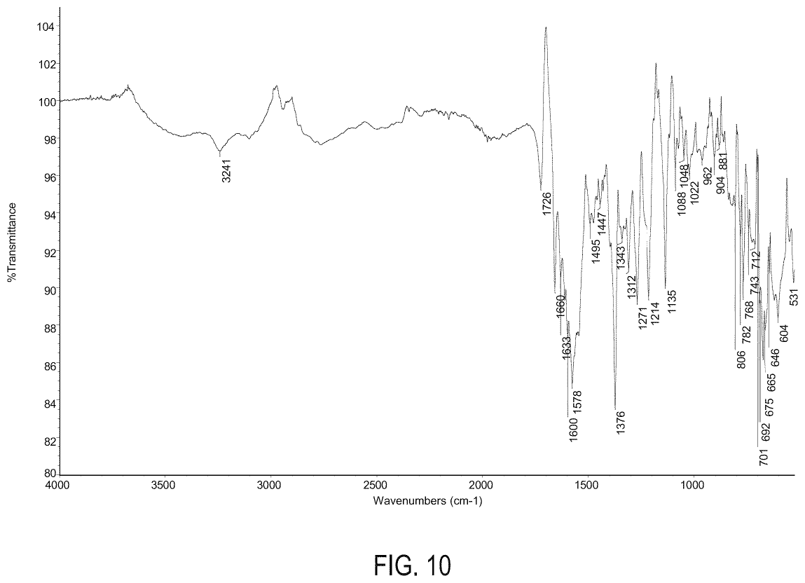

Enumerated embodiment 14A. The crystalline form of any of enumerated embodiments 1A-13A, wherein the crystalline form is characterized by an FTIR spectrum substantially as shown in FIG. 10.

Enumerated embodiment 15A. The crystalline form of any of enumerated embodiments 1A-14A, wherein the crystalline form is characterized by peaks at about 1600 cm-1 and about 1578 cm⁻¹ as determined by FTIR.

Enumerated embodiment 16A. The crystalline form of any of enumerated embodiments 1A-15A, wherein the crystalline form is characterized by peaks at about 1600 cm⁻¹, about 1578 cm⁻¹, and about 1376 cm⁻¹ as determined by FTIR.

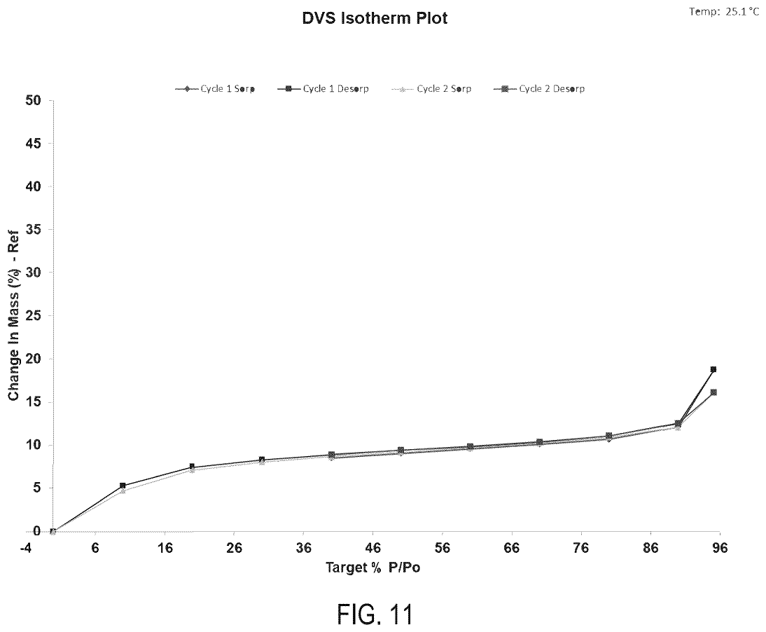

Enumerated embodiment 17A. The crystalline form of any of enumerated embodiments 1A-16A, wherein the crystalline form is characterized by DVS curve substantially as shown in FIG. 11.

Enumerated embodiment 18A. The crystalline form of any of enumerated embodiments 1A-17A, wherein the crystalline form is characterized by about 1.6% water update from about 40% RH to about 70% RH as determined by DVS.

Enumerated embodiment 19A. The crystalline form of any of enumerated embodiments 1A-18A, wherein the crystalline form is characterized by about 1.6% water update from about 40% RH to about 70% RH, and about 8.0% water uptake from about 80% RH to about 95% RH as determined by DVS.

Enumerated embodiment 20A. The crystalline form of any one of enumerated embodiments 1A-4A, wherein the crystalline form is characterized by an XRPD pattern substantially as shown in FIG. 4.

Enumerated embodiment 21A. The crystalline form of any one of enumerated embodiments 1A-4A or 20A, wherein the crystalline form is characterized by an XRPD pattern comprising:
a. peaks at angles 2-theta of 3.61±0.2 degrees and 7.34±0.2 degrees;
b. peaks at angles 2-theta of 3.61±0.2 degrees, 7.34±0.2 degrees, and 9.42±0.2 degrees; or
c. peaks at angles 2-theta of 3.61±0.2 degrees, 7.34±0.2 degrees, 9.42±0.2 degrees, and 15.76±0.2 degrees.

Enumerated embodiment 22A. The crystalline form of any one of enumerated embodiments 1A-4A or 20A-21A, wherein the crystalline form is characterized by an XRPD pattern comprising:
a. peaks at angles 2-theta of 19.28±0.2 degrees and 23.12±0.2 degrees;
b. peaks at angles 2-theta of 18.86±0.2 degrees, 19.28±0.2 degrees, and 23.12±0.2 degrees; or
c. peaks at angles 2-theta of 16.98±0.2 degrees, 18.86±0.2 degrees, 19.28±0.2 degrees, 23.12±0.2 degrees, and 25.53±0.2 degrees.

Enumerated embodiment 23A. The crystalline form of any one of enumerated embodiments 1A-4A or 20A-22A, wherein the crystalline form is characterized by an XRPD pattern comprising:
a. peaks at angles 2-theta of 9.42±0.2 degrees and 14.52±0.2 degrees;
b. peaks at angles 2-theta of 9.42±0.2 degrees, 13.42±0.2 degrees, and 14.52±0.2 degrees; or
c. peaks at angles 2-theta of 3.61±0.2 degrees, 9.42±0.2 degrees, 13.16±0.2 degrees, 13.42±0.2 degrees, and 14.52±0.2 degrees.

Enumerated embodiment 24A. The crystalline form of any one of enumerated embodiments 1A-4A or 20A-23A, wherein the crystalline form is characterized by a DSC graph substantially as shown in FIG. 5.

Enumerated embodiment 25A. The crystalline form of any one of enumerated embodiments 1A-4A or 20A-24A, wherein the crystalline form is characterized by endotherm peaks at about 60.5° C. and about 123.7° C., as determined by DSC.

Enumerated embodiment 26A. The crystalline form of any one of enumerated embodiments 1A-4A or 20A-25A, wherein the crystalline form is characterized by a TGA graph substantially as shown in FIG. 6.

Enumerated embodiment 27A. The crystalline form of any one of enumerated embodiments 1A-4A or 20A-26A, wherein the crystalline form is characterized by a weight loss of about 5.6% after heating from about 34° C. to about 80° C., as determined by TGA.

Enumerated embodiment 28A. The crystalline form of enumerated embodiment 27A, wherein the crystalline form is further characterized by a weight loss of about 1.7% after heating from about 80° C. to about 120° C., as determined by TGA.

Enumerated embodiment 29A. The crystalline form of any one of enumerated embodiments 1A-4A or 20A-28A, wherein the crystalline form has a water content of about 7.4% by weight.

Enumerated embodiment 30A. The crystalline form of any of any one of enumerated embodiments 1A-4A or 20A-29A, wherein the crystalline form is a mixed solvate of isopropyl alcohol and water.

Enumerated embodiment 31A. The crystalline form of any one of enumerated embodiments 1A-4A, wherein the crystalline form is characterized by an XRPD pattern substantially as shown in FIG. 7.

Enumerated embodiment 32A. The crystalline form of any one of enumerated embodiments 1A-4A or 31A, wherein the crystalline form is characterized by an XRPD pattern comprising peaks at angles 2-theta of 5.12±0.2 degrees and 6.21±0.2 degrees.

Enumerated embodiment 33A. The crystalline form of any one of enumerated embodiments 1A-4A or 31A-32A, wherein the crystalline form is characterized by an XRPD pattern comprising:
a. peaks at angles 2-theta of 20.45±0.2 degrees and 21.26±0.2 degrees;
b. peaks at angles 2-theta of 20.45±0.2 degrees, 21.26±0.2 degrees, and 24.97±0.2 degrees; or
c. peaks at angles 2-theta of 6.21±0.2 degrees, 20.45±0.2 degrees, 21.26±0.2 degrees, 24.97±0.2 degrees, and 26.43±0.2 degrees.

Enumerated embodiment 34A. The crystalline form of any one of enumerated embodiments 1A-4A or 31A-33A, wherein the crystalline form is characterized by an XRPD pattern comprising:
a. peaks at angles 2-theta of 6.21±0.2 degrees and 8.68±0.2 degrees;
b. peaks at angles 2-theta of 6.21±0.2 degrees, 8.68±0.2 degrees, and 10.14±0.2 degrees; or
c. peaks at angles 2-theta of 5.12±0.2 degrees, 6.21±0.2 degrees, 8.12±0.2 degrees, 8.68±0.2 degrees, and 10.14±0.2 degrees.

Enumerated embodiment 35A. The crystalline form of any one of enumerated embodiments 1A-4A or 31A-34A, wherein the crystalline form is characterized by a DSC graph substantially as shown in FIG. 8.

Enumerated embodiment 36A. The crystalline form of any one of enumerated embodiments 1A-4A or 31A-35A, wherein the crystalline form is characterized by an endotherm peak at about 58° C., as determined by DSC.

Enumerated embodiment 37A. The crystalline form of any one of enumerated embodiments 1A-4A or 31A-36A, wherein the crystalline form is characterized by a TGA graph substantially as shown in FIG. 9.

Enumerated embodiment 38A. The crystalline form of any of any one of enumerated embodiments 1A-4A or 31A-37A, wherein the crystalline form is characterized by a weight loss of about 8.3% after heating from about 30° C. to about 120° C., as determined by TGA.

Enumerated embodiment 39A. A method of preparing a crystalline form of (S)-4-((2-methoxyethyl)(4-(5,6,7,8-tetrahydro-1,8-naphthyridin-2-yl)butyl)amino)-2-(quinazolin-4-ylamino)butanoic acid, or a hydrate thereof, comprising preparing a mixture of (S)-4-((2-methoxyethyl)(4-(5,6,7,8-tetrahydro-1,8-naphthyridin-2-yl)butyl)amino)-2-(quinazolin-4-ylamino)butanoic acid and tartaric acid in a solvent.

Enumerated embodiment 40A. The method of enumerated embodiment 39A, wherein the solvent comprises an alcohol.

Enumerated embodiment 41A. The method of enumerated embodiment 40A, wherein the alcohol comprises methanol.

Enumerated embodiment 42A. The method of any one of enumerated embodiments 39A-41A, wherein the mixture is heated to about 50° C. for about 2 hours, and cooled to about 25° C.

Enumerated embodiment 43A. The method of enumerated embodiment 39A, wherein the alcohol comprises isopropanol.

Enumerated embodiment 44A. The method of enumerated embodiment 43A, wherein the mixture is stirred at about 22-27° C. for about 2 days.

Enumerated embodiment 45A. The method of any of enumerated embodiments 39A-44A, wherein the crystalline form is a cocrystal.

Enumerated embodiment 46A. A pharmaceutical composition comprising the crystalline form of any one of enumerated embodiments 1A-38A, and a pharmaceutically acceptable carrier or excipient.

Enumerated embodiment 47A. A kit comprising the crystalline form of any one of enumerated embodiments 1A-38A, or the pharmaceutical composition of enumerated embodiment 46A, and instructions for the treatment of a fibrotic disease.

Enumerated embodiment 48A. A method of treating a disease in an individual in need thereof comprising administering to the individual the crystalline form of any one of enumerated embodiments 1A-38A, or the pharmaceutical composition of enumerated embodiment 46A, wherein the disease is selected from the group consisting of fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, and Crohn's Disease.

Enumerated embodiment 49A. The method of enumerated embodiment 48A, wherein the disease comprises fibrosis.

Enumerated embodiment 50A. The method of enumerated embodiment 49A, wherein the fibrosis is pulmonary fibrosis, liver fibrosis, skin fibrosis, cardiac fibrosis, kidney fibrosis, gastrointestinal fibrosis, primary sclerosing cholangitis, or biliary fibrosis.

Enumerated embodiment 51A. The method of any of enumerated embodiments 49A, wherein the fibrotic disease is liver fibrosis, cardiac fibrosis, primary sclerosing cholangitis, or biliary fibrosis.

Enumerated embodiment 52A. The method of enumerated embodiment 48A or 49A, wherein the disease is progressive pulmonary fibrosis, pulmonary fibrosis associated with rheumatoid arthritis, idiopathic pulmonary fibrosis (IPF), radiation-induced pulmonary fibrosis, alcoholic liver disease induced fibrosis, or primary sclerosing cholangitis (PSC).

Enumerated embodiment 53A. A method of treating a disease in an individual in need thereof comprising administering to the individual the crystalline form of any one of enumerated embodiments 1A-38A, or the pharmaceutical composition of enumerated embodiment 46A, wherein the disease is liver fibrosis induced by pathogens, alcoholic steatosis induced liver fibrosis, cirrhosis, or biliary tract fibrosis.

Enumerated embodiment 54A. A method of treating a disease in an individual in need thereof comprising administering to the individual the crystalline form of any one of enumerated embodiments 1A-38A, or the pharmaceutical composition of enumerated embodiment 46A, wherein the disease is diabetic nephrosclerosis, hypertensive nephrosclerosis, or acute kidney injury from contrast induced nephropathy, glomerulonephritis, end-stage kidney disease, hearing loss, changes to the lens of the eye, hematuria, or proteinuria.

Enumerated embodiment 55A. A method of treating a disease in an individual in need thereof comprising administering to the individual the crystalline form of any one of enumerated embodiments 1A-38A, or the pharmaceutical composition of enumerated embodiment 46A, wherein the disease is scleroderma, systemic sclerosis, systemic and local sclerosis or scleroderma, keloids and hypertrophic scars, or post-surgical adhesions.

Enumerated embodiment 56A. A method of treating a disease in an individual in need thereof comprising administering to the individual the crystalline form of any one of enumerated embodiments 1A-38A, or the pharmaceutical composition of enumerated embodiment 46A, wherein the disease is atherosclerosis or restenosis, post myocardial infarction induced fibrosis, or inherited cardiomyopathy.

Enumerated embodiment 57A. The method of any of enumerated embodiments 48A-52A, wherein the fibrotic disease is idiopathic pulmonary fibrosis.

Enumerated embodiment 58A. The method of any of enumerated embodiments 48A-52A, wherein the fibrotic disease is progressive pulmonary fibrosis.

Enumerated embodiment 59A. The method of any of enumerated embodiments 48A-52A, wherein the fibrotic disease is primary sclerosing cholangitis.

Enumerated embodiment 60A. The method of enumerated embodiment 48A, wherein the disease is interstitial lung disease.

Enumerated embodiment 61A. The method of enumerated embodiment 48A, wherein the disease is rheumatoid arthritis associated interstitial lung disease.

Enumerated embodiment 62A. Use of the crystalline form of any one of enumerated embodiments 1A-38A in the manufacture of a medicament for the treatment of a fibrotic disease.

Enumerated embodiment 63A. Use of the crystalline form of any one of enumerated embodiments 1A-38A in the manufacture of a medicament for the treatment of a disease mediated by at least one of αᵥβ₁ integrin and αᵥβ₆ integrin.

Enumerated embodiment 64A. The crystalline form of any one of enumerated embodiments 1A-38A, or the pharmaceutical composition of enumerated embodiment 46A, for use in treating a fibrotic disease.

Enumerated embodiment 65A. The crystalline form of any one of enumerated embodiments 1A-38A, or the pharmaceutical composition of enumerated embodiment 46A, for use in treating a disease mediated by at least one of αᵥβ₁ integrin and αᵥβ₆ integrin.

### EXAMPLES

The following examples are provided to further aid in understanding the embodiments disclosed in the application and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

The following abbreviations may be used herein:

| | |
|---|---|
| XRPD | X-Ray Powder Diffraction |
| DSC | Differential Scanning Calorimetry |
| TGA | Thermogravimetric Analysis |
| DVS | Dynamic Vapor Sorption |
| equiv. or eq. | Equivalents |
| RH | Relative humidity |
| RT | Room temperature |
| MEK | Methyl ethyl ketone |
| IPAc | Isopropyl acetate |
| MIBK | 4-Methyl-2-pentanone |
| EtOH | Ethanol |
| FTIR | Fourier Transform Infrared spectroscopy |
| DMSO | Dimethyl sulfoxide |
| TBME or MTBE | tert-Butyl methyl ether |
| THF | Tetrahydrofuran |
| 2-MeTHF | 2-Methyltetrahydrofuran |
| CPME | Cyclopentyl methyl ether |
| DCM | Dichloromethane |
| MeOH | Methanol |
| ACN | Acetonitrile |
| IPA | Isopropyl alcohol |
| TFA | Trifluoroacetic Acid |
| API | Active Pharmaceutical Ingredient |
| HPLC | High performance liquid chromatography |

The crystalline forms were characterized by various analytical techniques, including XRPD, DSC, and TGA using the procedures described below.

### XRPD

### X-Ray Powder Diffractometer (XRPD)

| | |
|---|---|
| Instrument | Bruker D8 Advance |

Method 1 (used for FIG. 4 and FIG. 7)

| | |
|---|---|
| X-ray geometry | Reflection |
| Detector | LYNXEYE_XE_T (1D mode) |
| Open angle | 2.9° (max) |
| Radiation | Cu/K-Alpha1 (λ = 1.5406 Å) |
| X-ray generator power | 40 kV, 40 mA |
| Primary beam path slits | Twin_Primary motorized slit: 10.0 mm by |
| | sample length; Primary Soller slit: 2.5° |
| Secondary beam path slits | Secondary Soller slit: 2.5° |
| Scan mode | Continuous scan |
| Scan type | Locked coupled |
| Step size | 0.02° |
| Time per step | 0.12 second per step |
| Scan range | 3° to 40° |
| Sample rotation speed | 15 rpm |
| Sample holder | Monocrystalline silicon, flat surface |

Method 2 (used for FIG. 1)

| | |
|---|---|
| X-ray geometry | Reflection |
| Detector | LYNXEYE_XE_T (1D mode) |
| Open angle | 2.9° (max) |
| Radiation | Cu/K-Alpha1 (λ = 1.5406 Å) |
| X-ray generator power | 40 kV, 40 mA |
| Primary beam path slits | Twin_Primary motorized slit: 10.0 mm by |
| | sample length; Primary Soller slit: 2.5° |
| Secondary beam path slits | Secondary Soller slit: 2.5° |
| Scan mode | Continuous scan |
| Scan type | Locked coupled |
| Step size | 0.02° |
| Time per step | 0.4 second per step |
| Scan range | 2° to 40° |
| Sample rotation speed | 15 rpm |
| Sample holder | Flat monocrystalline silicon |

### DSC

### Differential Scanning Calorimetric (DSC)

| Instrument | TA Discovery 2500 or Q2000 | |
|---|---|---|
| Sample pan | Tzero pan and Tzero hermetic lid | |
| | with a pin hole of 0.7 mm in diameter | |
| Temperature range | 0 to 250° | C. |
| Heating rate | 10° | C./min |
| Nitrogen flow | 50 | mL/min |

| | |
|---|---|
| Sample mass | About 0.5-1.5 mg |

Modulated Differential Scanning Calorimeter (mDSC)

| Instrument | TA Instruments Discovery 2500 | |
|---|---|---|
| Sample pan | Tzero pan and Tzero hermetic lid with a manually | |
| | punched pin hole of about 0.7 mm in diameter | |
| Temperature range | 0 to 200° | C. |
| Modulation amplitude | ±0.32° | C./min |
| Period of modulation | 60 | s |
| Heating rate | 2° | C./min |
| Nitrogen flow | 50 | mL/min |
| Sample mass | ~2-10 | mg |

### TGA

### Thermal Gravimetric Analysis (TGA)

| Instrument | Discovery 5500 or Q5000 | |
|---|---|---|
| Sample pan | Aluminum, open | |
| Start temperature | Ambient condition (below 35° C.) | |
| Final temperature | 300° | C. |
| Heating rate | 10° | C./min |

| | |
|---|---|
| Nitrogen flow | Balance 10 mL/min; sample chamber 25 mL/min |
| Sample mass | About 2-10 mg |

### DVS

### Dynamic Vapor Sorption (DVS)

| | |
|---|---|
| Instrument | Surface Measurement Systems (SMS) Intrinsic |
| Temperature | 25° C. |
| Solvent | Water |
| Carrier Gas | Nitrogen, 200 ccm |
| and Flow Rate | |
| Sample mass | About 5-50 mg |

### Example 1. Preparation of Crude(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A

(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was prepared using the following procedure. 103 mg of the amorphous free form of (S)-4-((2-methoxyethyl)(4-(5,6,7,8-tetrahydro-1,8-naphthyridin-2-yl)butyl)amino)-2-(quinazolin-4-ylamino)butanoic acid and 61 mg (2.0 equivalents) of L-tartaric acid were weighed into an 8 mL glass vial. 0.4 mL of methanol (with 5% water by volume) was added into the vial at 50° C. and a suspension was obtained. After stirring at 50° C. for 2 hours, the suspension was cooled to 25° C. and kept stirring at 25° C. 3 mL of methanol (with 5% water by volume) was added because the suspension became thick. This suspension is referred to as “suspension 1.” After stirring at 25° C. for 3 days, the sample was investigated by XRPD. XRPD showed L-tartaric acid cocrystal Pattern A was obtained.

Further Pattern A material was prepared as follows. 8.3 g of the amorphous free form of (S)-4-((2-methoxyethyl)(4-(5,6,7,8-tetrahydro-1,8-naphthyridin-2-yl)butyl)amino)-2-(quinazolin-4-ylamino)butanoic acid and 4.8 g (2.0 equivalents) of L-tartaric acid were weighed into a 100 mL glass vial. 35 mL of methanol (with 5% water by volume) was added into the vial at 50° C. A sticky sample was obtained. Then 1 mL of suspension 1 was added into the above mixture as seeds. After stirring at 50° C. for about 5 min, the sticky sample converted to a hazy suspension. After stirring at 50° C. for 20 min, another 1 mL of suspension 1 was added into above hazy suspension. After stirring at 50° C. for 40 min, the hazy suspension converted to a suspension. A further 1 mL of suspension 1 was added. 50 mL of methanol (with 5% water by volume) was added because the suspension became thick. After stirring at 50° C. for 2 h, the suspension was cooled to 25° C. and kept stirring at 25° C. for about 3 days. Solids were collected by centrifugation and then dried at 50° C. under vacuum for about 2 h. 13.5 g of the (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was obtained as an off-white solid in 94% yield.

### Example 2. Dried(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A

Since (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A prepared according to Example 1 contains about 2.3% MeOH residue by weight (0.7 equivalent by molar ratio), vacuum drying was applied to remove residual methanol. 200 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was dried under vacuum conditions at 25° C. for about 30 min and then at 50° C. for about 1.5 h. The dried solid was investigated by XRPD and GC. GC showed no detectable residual methanol. XRPD showed no form change.

The obtained(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was investigated using a melting point instrument. Based on observation, the melting peak of Pattern A is about 120° C. Decomposition occurs from about 150° C.

The dried(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was also investigated by VT-XRPD experiments. The(S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was heated to 110° C. under dry nitrogen flow for about 30 min and then cooled to ambient condition (20-26° C., 20%-35% RH). The obtained solid parts were investigated by XRPD. XRPD showed no form change.

### Example 3. Enhanced crystallinity of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A

To improve the crystallinity of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A, equilibration under a temperature cycle between 5° C. to 50° C. at a heating/cooling rate of 0.1° C./min was applied to the compound.

600 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was equilibrated in 1.8 mL of isopropanol/water (89:11, v:v) under a temperature cycle between 5° C. to 50° C. at a heating/cooling rate of 0.1° C./min. A suspension was obtained. After equilibration for 3 days, 1 mL of isopropanol/water (89:11, v:v) was added to above suspension because the suspension became thick.

After equilibration for 16 days, solids were collected by centrifugation and dried at 50° C. under vacuum for about 2 h. 500 mg of the (S)-4-((2-methoxyethyl)(4-(5,6,7,8- tetrahydro-1,8-naphthyridin-2-yl)butyl)amino)-2-(quinazolin-4-ylamino)butanoic acid L-tartaric acid cocrystal Pattern A was obtained as an off-white solid in 83% yield.

Properties of the enhanced crystallinity cocrystal Pattern A material are shown in Table 4.

TABLE 4

| Parameter | Method | Result |
|---|---|---|
| Purity | HPLC | 316 nm: 99.4% |
| X-ray diffraction | 2-40° (2 theta) | High crystallinity |
| Thermal events | DSC, 10° C./min | Dehydration from about 8° C.; |
| and enthalpy | | melting Tₒₙₛₑₜ 128.3° C., |
| | | enthalpy about 46 J/g |
| Thermogravimetry | TGA, 10° C./min | About 8.0% at 120° C. |
| Stoichiometry | ¹H-NMR | Free form: L-tartaric acid = |
| | (DMSO-d6) | 1:2.0 (mole:mole) |
| Residual solvent(s) | ¹H-NMR | 0.4% IPA residue by weight |
| | (DMSO-d6) | (0.06 equiv. by molar ratio) |
| Water content (for | Karl Fischer | 9.2% of water by weight (4.5 |
| hydrate) | (coulometric) | equivalents by molar ratio) |
| Morphology | Polarized light | Aggregated irregular particles, |
| | microscopy | ranging from about 1 μm to |
| | (PLM) | 80 μm |

### Example 4. Preparation of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B

Method 1: (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B was prepared using the following procedure. 200 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A (see Example 1) was dissolved in 200 μL of water at ambient temperature (about 22-27° C.). The obtained thin suspension was filtered through a 0.45 μm syringe membrane filter to obtain a clear solution. 200 μL of IPA was added into the clear solution slowly, and a hazy suspension was obtained. About 10 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B seeds were added into the above hazy suspension. 400 μL of IPA was added to the hazy suspension slowly, and then this hazy suspension converted to a suspension. After stirring at room temperature (about 22-27° C.) for about 4 h, 400 μL of IPA was added into the above suspension because the suspension became thick. After stirring at 25° C. for about 2 days, solids were collected by centrifugation and dried under ambient conditions (21-26° C., 55%-65% RH) for about 24 h. (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B was obtained as an off-white solid.

Method 2: (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B was also prepared using the following procedure. 500 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A (see Example 1) was dissolved in 500 μL of water at ambient temperature (about 22-27° C.). The obtained thin suspension was filtered through a 0.45 μm syringe membrane filter to obtain a clear solution. About 60 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B seeds were added into the above clear solution. Then 1.5 mL of IPA was added into the above mixture slowly, and a suspension was obtained. After stirring at 25° C. for about 2 h, 4 mL of IPA was added into above suspension because the suspension became thick (suspension).

After stirring at 25° C. for about 2 days, solids were collected by centrifugation and dried under ambient conditions (20-26° C., 42%-62% RH) for about 24 h. The obtained solids were then dried under vacuum at 25° C. for about 2 h and investigated by XRPD. Pattern B was obtained.

Properties of the cocrystal Pattern B material are shown in Table 5.

TABLE 5

| Parameter | Method |
|---|---|
| Crystallinity | Medium crystallinity |
| (by XRPD) | |
| Thermal events | Dehydration from about 1° C.; dehydration |
| (by DSC or m DSC) | upon melting from about 94° C. |
| Weight loss | About 5.6% @ 80° C., about 1.7% from 80° C. |
| (by TGA) | to 120° C. |
| Stoichiometric | Free form: L- tartaric acid = 1:2.0 (mole:mole) |
| ratio | |
| (by ¹H-NMR) | |
| Residual solvent | ¹H-NMR: 0.3% IPA residue by weight (0.05 equiv. |
| (by ¹H-NMR) | by molar ratio) |
| Water content | 7.4% of water by weight (3.5 equivalents |
| (by KF) | by molar ratio) |
| Comments | Pattern B melts from about 90° C. and |
| | decomposes from about 130° C. based on |
| | observation by melting point. |

### Example 5. Preparation of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C

Method 1: (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C was prepared using the following procedure. 200 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A (see Example 1) was dissolved in 1.8 mL of IPA/water (7:3, v:v) at 50° C. The obtained thin suspension was filtered through a 0.45 μm syringe membrane filter to obtain a clear solution.

About 0.1 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C seeds were added into above clear solution, and the mixture was cooled to 5° C. at 0.1° C./min. After stirring at 5° C. for about 2 days, a suspension was obtained. Solids were collected by centrifugation and then placed in a chamber with relative humidity of 10%-20% RH at room temperature (21-26° C.) for about 24 h. (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C was obtained as an off-white solid.

Method 2: (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern C was prepared using the following procedure. 200 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A (see Example 1) was dissolved in 1.8 mL of IPA/water (7:3, v:v) at 50° C. The obtained thin suspension was filtered through a 0.45 μm syringe membrane filter to obtain a clear solution. The clear solution was cooled to 5° C. at 0.1° C./min after stirring at 50° C. for about 30 min. After stirred at 5° C. for about 2 days, a suspension was obtained. Solids were collected by centrifugation and investigated by XRPD. A low crystallinity Pattern C was obtained.

The suspension was kept stirring at 5° C. for another 3 days. Solids were collected by centrifugation and investigated by XRPD. A low crystalline Pattern B was obtained.

### Example 6. Differential scanning calorimetry of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal

DSC of Pattern A prepared in Example 3 shows a dehydration peak from about 8° C. for (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A; see FIG. 2. at the crystal has a phase transition Tₒₙₛₑₜ of 128.3° C. with an enthalpy of about 46 J/g. TGA shows about 8.0% weight loss at about 120° C. ¹H-NMR shows stoichiometry of (S)-4-((2-methoxyethyl)(4-(5,6,7,8-tetrahydro-1,8-naphthyridin-2-yl)butyl)amino)-2-(quinazolin-4-ylamino)butanoic acid free form: L-tartaric acid is 1:2.0 (molar ratio) and 0.4% IPA residue by weight (0.06 equiv. by molar ratio). Polarized light microscopy (PLM) shows the sample consists of aggregated irregular particles with particle size ranging from about 1 μm to 80 μm.

Example 7. Fourier transform infrared spectroscopy of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A

A Fourier transform infrared (FTIR) spectrum was recorded for (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A. The spectrum was taken on a Thermo Scientific Nicolet iS5 spectrometer in attenuated total reflectance (ATR) mode, using 32 scans. The sample used was a solid powder of cocrystal Pattern A. The resulting spectrum is shown in FIG. 10.

Example 8. Dynamic vapor sorption results for (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A

Dynamic vapor sorption of Pattern A was studied using a Surface Measurement Systems (SMS) Intrinsic instrument. Total gas flow was 200 sccm. The oven temperature was 25° C. Water was used as the solvent, in a cycle of 40-95-0-95-40% RH. The stage step was 10% RH and equilibrium was within 0.002 dm/dt (%/min) (minimum dm/dt stability duration: 60 min; maximum dm/dt stage time: 360 min). The sample mass was about 5-50 mg. Results are shown in FIG. 11.

### Example 9. Study of Interrelationship of Polymorphs Using Water Activity Experiments

The relative stability of the hydrate Pattern A and the hydrate Pattern B was investigated using water activity experiments in MeOH/water systems. About 5 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A and 5 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern B were added to 0.1-0.5 mL saturated solutions of the methanol/water system. Obtained suspensions were stirred at 5° C., 25° C. and 50° C. for up to 21 days, respectively. Solid parts (wet cakes) were isolated by centrifugation filtration and investigated by XRPD.

As summarized in Table 6 below, Pattern A was obtained with a.w. ≤0.8 at 5° C., a.w. ≤0.8 at 25° C., and a.w. ≤1.0 at 50° C. Pattern B was obtained when a.w. was 1.0 at 5° C. and 25° C.

TABLE 6

XRPD

| Solvents | 5° C. | 25° C. | 50° C. |
|---|---|---|---|
| Methanol | 7 days: | 7 days: | 7 days: |
| (a.w. = 0)* | Pattern A | Pattern A | Pattern A |
| Methanol/water | 7 days: | 7 days: | 7 days: |
| (a.w. = 0.2)* | Pattern A | Pattern A | Pattern A |
| Methanol/water | 7 days: | 7 days: | 7 days: |
| (a.w. = 0.4)* | Pattern A | Pattern A | Pattern A |
| Methanol/water | 7 days: | 7 days: | 7 days: |
| (a.w. = 0.6)* | Pattern A | Pattern A | Pattern A |
| Methanol/water | 13 days: | 7 days: | 7 days: |
| (a.w. = 0.8)* | Pattern A | Pattern A | Pattern A |
| Water | 21 days: | 21 days: | 7 days: |
| (a.w. = 1.0)* | Pattern B | Pattern B | Pattern A |

Example 10. Evaluation of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A and comparison with the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid

This example demonstrates the evaluation of the bulk stability, solubility, hygroscopicity, and mechanic properties of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A as obtained from Example 3, including comparisons to the stability and solubility of the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid (see U.S. Pat. No. 12,018,025, US Pat. Appl. Publ. No. 2022/0177468, and International Pat. Publ. No. WO 2022/109598, the contents of which are hereby incorporated by reference in their entirety).

Example 10A. Bulk stability of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A in comparison with phosphate salt of (S)-4-((2-methoxyethyl)(4-(5,6,7,8-tetrahydro-1,8-naphthyridin-2-yl)butyl)amino)-2-(quinazolin-4-ylamino)butanoic acid

This example demonstrates the bulk stability under varying conditions of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A relative to phosphate salt of (S)-4-((2-methoxyethyl)(4-(5,6,7,8-tetrahydro-1,8-naphthyridin-2-yl)butyl)amino)-2-(quinazolin-4-ylamino)butanoic acid. Bulk stability of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was evaluated at 25° C./92.5% RH in an open container, at 40° C./75% RH in an open container, and at 60° C. in a closed container over 1 week and 4 weeks. As summarized in Table 7 below, (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A exhibits higher stability than the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid under various temperatures and relative humidities (the three bulk stability conditions BS1, BS2, BS3). After being stressed in high humidity and at high temperature with high humidity, the L-tartaric acid cocrystal Pattern A remained the same form. The L-tartaric acid cocrystal Pattern A showed 0.9%, 2.1%, and 0.9% purity decrease after being stressed at 25° C./92.5% RH in an open container, at 40° C./75% RH in an open container, and at 60° C. in a closed container for 4 weeks, respectively. The phosphate salt of (S)-4-((2-methoxyethyl)(4-(5,6,7,8-tetrahydro-1,8-naphthyridin-2-yl)butyl)amino)-2-(quinazolin-4-ylamino)butanoic acid showed 0.6%, 2.5%, and 1.1% purity decrease after being stressed at 25° C./92.5% RH in an open container, at 40° C./75% RH in an open container, and at 60° C. in a closed container for 4 weeks, respectively.

After being stressed in high humidity and at high temperature with high humidity (stressed at 25° C./92.5% RH in an open container, at 40° C./75% RH in an open container, and at 60° C. in a closed container), the L-tartaric acid cocrystal Pattern A remained the same form after four weeks. The L-tartaric acid cocrystal Pattern A was physically stable at 25° C./92.5% RH in an open container, at 40° C./75% RH in an open container and at 60° C. in a closed container over 4 weeks. The phosphate salt of (S)-4-((2-methoxyethyl)(4-(5,6,7,8-tetrahydro-1,8-naphthyridin-2-yl)butyl)amino)-2-(quinazolin-4-ylamino)butanoic acid was physically stable after being stressed at 60° C. in a closed container over 4 weeks. However, it became deliquescent after being stressed at 25° C./92.5% RH in an open container and at 40° C./75% RH in an open container after 1 week.

TABLE 7

| | | Physical Form | | | | | | | |
|---|---|---|---|---|---|---|---|---|---|
| | | L-Tartaric acid cocrystal Pattern A, | | | | | | | |
| | | hydrate | | | | Phosphate salt, hydrate | | | |
| Exp. ID | Initial | 316 nm: 99.4% | | | | 316 nm: 99.8% | | | |
| | purity by | | | | | | | | |
| | HPLC | | | | | | | | |
| | Initial color | Off-white | | | | Off-white | | | |
| | Solid state, 25° C./92.5% RH, open container | | | | | | | | |
| | | 1 week | | 4 weeks | | 1 week | | 4 weeks | |
| BS1 | Bulk | 316 nm: | No color | 316 | No color | 316 | Delique | 316 | Delique |
| | (HPLC) | 99.2% | change | nm: | change | nm: | scent | nm: | scent |
| | | | | 98.5% | | 99.7% | | 99.2% | |
| | Bulk | No form change | | No form change | | Not carried out. | | Not carried out. | |
| | (XRPD) | | | | | | | | |
| | Solid state, 40° C./75% RH, open container | | | | | | | | |
| | | 1 week | | 4 weeks | | 1 week | | 4 weeks | |
| BS2 | Bulk | 316 nm: | No color | 316 | No color | 316 | Delique | 316 | Delique |
| | (HPLC) | 98.9% | change | nm: | change | nm: | scent | nm: | scent |
| | | | | 97.3% | | 98.7% | | 97.3% | |
| | Bulk | No form change | | No form change | | Not carried out. | | Not carried out. | |
| | (XRPD) | | | | | | | | |
| | Solid state, 60° C., closed container | | | | | | | | |
| | | 1 week | | 4 weeks | | 1 week | | 4 weeks | |
| BS3 | Bulk | 316 nm: | No color | 316 | No color | 316 | No color | 316 | No color |
| | (HPLC) | 99.1% | change | nm: | change | nm: | change | nm: | change |
| | | | | 98.5% | | 99.3% | | 98.7% | |

| | | | | |
|---|---|---|---|---|
| Bulk | No form change | No form change | No form change | No form change but |
| (XRPD) | | | | crystallinity |
| | | | | decreased |

Example 10B. Solubility of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A in comparison with the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid

This example demonstrates the solubility under varying conditions of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A versus the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid. The solubilities are shown in Table 8 below.

To prepare the samples of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A, 80.6 mg was weighed into a 2 mL glass vial. Next, 0.18 mL of solubility medium was added. The salt amount used is equivalent to 45 mg of the amorphous free form. Obtained suspensions/solutions were stirred at 37° C. at 400 rpm and sampled at 2 h and at 24 h. The samples were centrifuged at 37° C. at 14,000 rpm for 5 min. Supernatants were analyzed by HPLC and pH meter for solubility and pH value, respectively.

To prepare the samples of the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid, 625 mg was weighed into a 4 mL glass vial, and solubility medium was added (FaSSGF=fasted state simulated gastric fluid; FaSSIF=fasted state simulated intestinal fluid; FeSSIR=fed state simulated intestinal fluid). The obtained suspensions/solutions were stirred at 37° C. at 400 rpm and sampled at 2 h and at 24 h. The samples were centrifuged at 37° C. at 14,000 rpm for 5 min. Supernatants were analyzed by HPLC and pH meter for solubility and pH value, respectively. Residual solids (wet cakes) were characterized by XRPD to determine physical form.

TABLE 8

| | Solubility at 37° C., target concentration 250 mg/mL (in free form), equilibration for 24 | | | | | | |
|---|---|---|---|---|---|---|---|
| | hours, LOQ: 0.03 mg/mL or 0.001 mg/mL | | | | | | |
| | | Physical Form | | | | | |
| | | L-Tartaric acid cocrystal | | | | | |
| | | Pattern A, hydrate | | | Phosphate salt, hydrate | | |
| | | Solubility | | XRPD | Solubility | | XRPD |
| | | (mg/ mL) | | of | (mg/ mL) | | of |
| | | | 24 h | residual | | 24 h | residual |
| Exp. ID | Solubility media | 2 h | (pH) | solid | 2 h | (pH) | solid |
| 11B-1 | pH 1.2 HCl buffer | 215.0 | 222.1 | // | >208 | >208 | // |
| | | | (1.4) | | | (1.6) | |
| 11B-2 | pH 4.5 acetate buffer | 214.2 | 220.5 | // | >228 | >228 | // |
| | (50 mM) | | (4.4) | | | (4.8) | |
| 11B-3 | pH 6.8 phosphate | 28.4 | 56.4 | Layered; | >229 | >229 | // |
| | buffer | | (6.8) | ¹H-NMR of | | (6.7) | |
| | (50 mM) | | | oil: free | | | |
| | | | | form | | | |
| 11B-4 | Water | 207.2 | 217.8 | // | >247 | >247 | // |
| | | | (3.2) | | | (5.8) | |
| 11B-5 | FaSSGF, pH 1.6 | 206.2 | 224.3 | // | >247 | >247 | // |
| | | | (1.6) | | | (5.8) | |
| 11B-6 | FaSSIF-v1, pH 6.5 | 84.1 | 63.2 | Layered; | >213 | >214 | // |
| | | | (6.6) | ¹H-NMR of | | (5.9) | |
| | | | | oil: free | | | |
| | | | | form | | | |
| 11B-7 | FeSSIF-v1, pH 5.0 | 203.3 | 216.9 | // | >214 | >218 | // |
| | | | (5.0) | | | (5.8) | |

“//”: Not carried out because sufficient solid was not obtained.

### Example 10C. Hygroscopicity of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A

The water sorption and desorption behavior of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was investigated by DVS at 25° C. with a cycle of 40-95-0-95-40% RH. XRPD was measured after the DVS test to determine form change. As summarized in Table 9 below, (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was found to be slightly hygroscopic at 70% RH and below, with about 1.6% water uptake from 40% RH to 70% RH. (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A became moderately hygroscopic in 80% RH and above with about 8.0% water uptake from 80% RH to 95% RH. It dehydrated in 10% RH and below. XRPD following the DVS experiments showed that no form change occurred.

TABLE 9

| | Method | | | |
|---|---|---|---|---|
| | 40-95-0-95-40% RH, dm/dt 0.002, minimum | | | |
| | equilibration time 60 min/step, maximum | | | |
| | equilibration time 360 min/step, 25° C. | | | |
| Relative | 1^{st} sorp. | 1^{st} desorp. | 2^{nd} sorp. | 2^{nd} desorp. |
| humidity | Weight % | Weight % | Weight % | Weight % |
| at 25° C. | change | change | change | change |
| 0% | // | 0.0 | 0.0 | // |
| 10% | // | 5.2 | 4.7 | // |
| 20% | // | 7.4 | 7.1 | // |
| 30% | // | 8.3 | 8.0 | // |
| 40% | 8.5 | 8.9 | 8.7 | 8.9 |
| 50% | 9.0 | 9.4 | 9.2 | 9.4 |
| 60% | 9.6 | 9.8 | 9.7 | 9.9 |
| 70% | 10.1 | 10.4 | 10.2 | 10.4 |
| 80% | 10.7 | 11.1 | 10.9 | 11.1 |
| 90% | 12.2 | 12.6 | 12.1 | 12.6 |
| 95% | 18.7 | 18.7 | 16.1 | 16.1 |

### Example 10D. Mechanic properties of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A

This example illustrates the stability of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A in several stress tests. (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was subjected to compression, dry grinding simulation, and wet granulation simulation.

### Example 10D1. Compression Study

About 20 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was compressed for 5 minutes under 2 MPa, 5 MPa and 10 MPa with a hydraulic press. Potential form change and degree of crystallinity were evaluated by XRPD. As summarized in Table 10 below, no form change was observed, and there was only a slight decrease in crystallinity as evidenced by broadening of peaks.

TABLE 10

| | |
|---|---|
| Pressure | XRPD |

| | | |
|---|---|---|
| 2 | MPa | Pattern A |
| 5 | MPa | Pattern A |
| 10 | MPa | Pattern A |

### Example 10D2. Dry Grinding

About 20 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A was ground manually with a mortar and a pestle for 3 min. Potential form change and degree of crystallinity were evaluated by XRPD. No form change was observed, and there was no obvious change in crystallinity.

### Example 10D3. Wet Granulation Simulation

Water or ethanol was added dropwise to about 20 mg of (S)-4-((2-methoxyethyl)(4-(5,6,7,8-tetrahydro-1,8-naphthyridin-2-yl)butyl)amino)-2-(quinazolin-4-ylamino)butanoic acid L-tartaric acid cocrystal Pattern A until the sample was wetted sufficiently. The wet sample was ground gently using a mortar and pestle. Post granulation sample was dried under ambient conditions for 30 min. The potential form change and degree of crystallinity were evaluated by XRPD. As summarized in Table 11 below, no form change was observed in either the water or ethanol granulation solvents. There was a slight decrease in crystallinity when using the water granulation solvent, and no obvious change in crystallinity when using the ethanol granulation solvent.

TABLE 11

| Granulation solvent | XRPD | Comments |
|---|---|---|
| Water | Pattern A | Slight decrease in crystallinity |
| Ethanol | Pattern A | No obvious change in |
| | | crystallinity |

### Example 11

Micro Electron Diffraction (Micro ED) experiments were carried out on the Pattern A crystalline form. For both (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid molecules, bond length normalization of C═O and C—O in the carboxylic acid functional groups was not observed in the crystal structure. Also, no proton transfer was observed from the carboxylic acid functional group of L-tartaric acid to the free form of (S)-4-((2-methoxyethyl)(4-(5,6,7,8-tetrahydro-1,8-naphthyridin-2-yl)butyl)amino)-2-(quinazolin-4-ylamino)butanoic acid. Therefore, the (S)-4-((2-methoxyethyl)(4-(5,6,7,8-tetrahydro-1,8-naphthyridin-2-yl)butyl)amino)-2-(quinazolin-4-ylamino)butanoic acid L-tartrate complex Pattern A was determined to be a cocrystal of (S)-4-((2-methoxyethyl)(4-(5,6,7,8-tetrahydro-1,8-naphthyridin-2-yl)butyl)amino)-2-(quinazolin-4-ylamino)butanoic acid with L-tartaric acid in a 1:2 molar ratio.

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A crystalline form of (S)-4-((2-methoxyethyl)(4-(5,6,7,8-tetrahydro-1,8-naphthyridin-2-yl)butyl)amino)-2-(quinazolin-4-ylamino)butanoic acid and L-tartaric acid, or a solvate thereof.

2. The crystalline form of claim 1, wherein the crystalline form is a cocrystal.

3. The cocrystal of claim 2, wherein the molar ratio of (S)-4-((2-methoxyethyl)(4-(5,6,7,8-tetrahydro-1,8-naphthyridin-2-yl)butyl)amino)-2-(quinazolin-4-ylamino)butanoic acid to L-tartaric acid is about 1:2.

4. The crystalline form of any one of claims 1-3, wherein the crystalline form is a solvate and the solvate is a hydrate.

5. The crystalline form of any one of claims 1-4, wherein the crystalline form is characterized by an XRPD pattern substantially as shown in FIG. 1.

6. The crystalline form of any one of claims 1-5, wherein the crystalline form is characterized by an XRPD pattern comprising:
a. peaks at angles 2-theta of 8.11±0.2 degrees and 10.93±0.2 degrees;
b. peaks at angles 2-theta of 8.11±0.2 degrees, 10.93±0.2 degrees, and 14.91±0.2 degrees; or
c. peaks at angles 2-theta of 8.11±0.2 degrees, 10.93±0.2 degrees, 14.91±0.2 degrees, and 22.46±0.2 degrees.

7. The crystalline form of any one of claims 1-6, wherein the crystalline form is characterized by an XRPD pattern comprising:
a. peaks at angles 2-theta of 6.94±0.2 degrees and 22.46±0.2 degrees;
b. peaks at angles 2-theta of 6.94±0.2 degrees, 10.93±0.2 degrees, and 22.46±0.2 degrees; or
c. peaks at angles 2-theta of 6.94±0.2 degrees, 8.47±0.2 degrees, 10.93±0.2 degrees, 22.46±0.2 degrees, and 24.78±0.2 degrees.

8. The crystalline form of any one of claims 1-7, wherein the crystalline form is characterized by an XRPD pattern comprising:
a. peaks at angles 2-theta of 6.94±0.2 degrees and 10.93±0.2 degrees;
b. peaks at angles 2-theta of 6.94±0.2 degrees, 8.47±0.2 degrees, and 10.93±0.2 degrees; or
c. peaks at angles 2-theta of 6.94±0.2 degrees, 8.47±0.2 degrees, 10.93±0.2 degrees, 14.45±0.2 degrees, and 14.91±0.2 degrees.

9. The crystalline form of any one of claims 1-8, wherein the crystalline form is characterized by a DSC graph substantially as shown in FIG. 2.

10. The crystalline form of any one of claims 1-9, wherein the crystalline form is characterized by endotherm peaks at about 74.7° C. and about 139.9° C., as determined by DSC.

11. The crystalline form of any one of claims 1-10, wherein the crystalline form is characterized by a TGA graph substantially as shown in FIG. 3.

12. The crystalline form of any one of claims 1-11, wherein the crystalline form is characterized by a weight loss of about 8% after heating from about 34° C. to about 120° C., as determined by TGA.

13. The crystalline form of any one of claims 1-12, wherein the crystalline form has a water content of about 9.2% by weight.

14. The crystalline form of any one of claims 1-13, wherein the crystalline form is characterized by an FTIR spectrum substantially as shown in FIG. 10.

15. The crystalline form of any one of claims 1-14, wherein the crystalline form is characterized by peaks at about 1600 cm⁻¹ and about 1578 cm⁻¹ as determined by FTIR.

16. The crystalline form of any one of claims 1-15, wherein the crystalline form is characterized by peaks at about 1600 cm⁻¹, about 1578 cm⁻¹, and about 1376 cm⁻¹ as determined by FTIR.

17. The crystalline form of any one of claims 1-16, wherein the crystalline form is characterized by DVS curve substantially as shown in FIG. 11.

18. The crystalline form of any one of claims 1-17, wherein the crystalline form is characterized by about 1.6% water update from about 40% RH to about 70% RH as determined by DVS.

19. The crystalline form of any one of claims 1-18, wherein the crystalline form is characterized by about 1.6% water update from about 40% RH to about 70% RH, and about 8.0% water uptake from about 80% RH to about 95% RH as determined by DVS.

20. The crystalline form of any one of claims 1-4, wherein the crystalline form is characterized by an XRPD pattern substantially as shown in FIG. 4.

21. The crystalline form of any one of claim 1-4 or 20, wherein the crystalline form is characterized by an XRPD pattern comprising:
a. peaks at angles 2-theta of 3.61±0.2 degrees and 7.34±0.2 degrees;
b. peaks at angles 2-theta of 3.61±0.2 degrees, 7.34±0.2 degrees, and 9.42±0.2 degrees; or
c. peaks at angles 2-theta of 3.61±0.2 degrees, 7.34±0.2 degrees, 9.42±0.2 degrees, and 15.76±0.2 degrees.

22. The crystalline form of any one of claim 1-4 or 20-21, wherein the crystalline form is characterized by an XRPD pattern comprising:
a. peaks at angles 2-theta of 19.28±0.2 degrees and 23.12±0.2 degrees;
b. peaks at angles 2-theta of 18.86±0.2 degrees, 19.28±0.2 degrees, and 23.12±0.2 degrees; or
c. peaks at angles 2-theta of 16.98±0.2 degrees, 18.86±0.2 degrees, 19.28±0.2 degrees, 23.12±0.2 degrees, and 25.53±0.2 degrees.

23. The crystalline form of any one of claim 1-4 or 20-22, wherein the crystalline form is characterized by an XRPD pattern comprising:
a. peaks at angles 2-theta of 9.42±0.2 degrees and 14.52±0.2 degrees;
b. peaks at angles 2-theta of 9.42±0.2 degrees, 13.42±0.2 degrees, and 14.52±0.2 degrees; or
c. peaks at angles 2-theta of 3.61±0.2 degrees, 9.42±0.2 degrees, 13.16±0.2 degrees, 13.42±0.2 degrees, and 14.52±0.2 degrees.

24. The crystalline form of any one of claim 1-4 or 20-23, wherein the crystalline form is characterized by a DSC graph substantially as shown in FIG. 5.

25. The crystalline form of any one of claim 1-4 or 20-24, wherein the crystalline form is characterized by endotherm peaks at about 60.5° C. and about 123.7° C., as determined by DSC.

26. The crystalline form of any one of claim 1-4 or 20-25, wherein the crystalline form is characterized by a TGA graph substantially as shown in FIG. 6.

27. The crystalline form of any one of claim 1-4 or 20-26, wherein the crystalline form is characterized by a weight loss of about 5.6% after heating from about 34° C. to about 80° C., as determined by TGA.

28. The crystalline form of claim 27, wherein the crystalline form is further characterized by a weight loss of about 1.7% after heating from about 80° C. to about 120° C., as determined by TGA.

29. The crystalline form of any one of claim 1-4 or 20-28, wherein the crystalline form has a water content of about 7.4% by weight.

30. The crystalline form of any of any one of claim 1-4 or 20-29, wherein the crystalline form is a mixed solvate of isopropyl alcohol and water.

31. The crystalline form of any one of claims 1-4, wherein the crystalline form is characterized by an XRPD pattern substantially as shown in FIG. 7.

32. The crystalline form of any one of claim 1-4 or 31, wherein the crystalline form is characterized by an XRPD pattern comprising peaks at angles 2-theta of 5.12±0.2 degrees and 6.21±0.2 degrees.

33. The crystalline form of any one of claim 1-4 or 31-32, wherein the crystalline form is characterized by an XRPD pattern comprising:
a. peaks at angles 2-theta of 20.45±0.2 degrees and 21.26±0.2 degrees;
b. peaks at angles 2-theta of 20.45±0.2 degrees, 21.26±0.2 degrees, and 24.97±0.2 degrees; or
c. peaks at angles 2-theta of 6.21±0.2 degrees, 20.45±0.2 degrees, 21.26±0.2 degrees, 24.97±0.2 degrees, and 26.43±0.2 degrees.

34. The crystalline form of any one of claim 1-4 or 31-33, wherein the crystalline form is characterized by an XRPD pattern comprising:
a. peaks at angles 2-theta of 6.21±0.2 degrees and 8.68±0.2 degrees;
b. peaks at angles 2-theta of 6.21±0.2 degrees, 8.68±0.2 degrees, and 10.14±0.2 degrees; or
c. peaks at angles 2-theta of 5.12±0.2 degrees, 6.21±0.2 degrees, 8.12±0.2 degrees, 8.68±0.2 degrees, and 10.14±0.2 degrees.

35. The crystalline form of any one of claim 1-4 or 31-34, wherein the crystalline form is characterized by a DSC graph substantially as shown in FIG. 8.

36. The crystalline form of any one of claim 1-4 or 31-35, wherein the crystalline form is characterized by an endotherm peak at about 58° C., as determined by DSC.

37. The crystalline form of any one of claim 1-4 or 31-36, wherein the crystalline form is characterized by a TGA graph substantially as shown in FIG. 9.

38. The crystalline form of any of any one of claim 1-4 or 31-37, wherein the crystalline form is characterized by a weight loss of about 8.3% after heating from about 30° C. to about 120° C., as determined by TGA.

39. A method of preparing a crystalline form of (S)-4-((2-methoxyethyl)(4-(5,6,7,8-tetrahydro-1,8-naphthyridin-2-yl)butyl)amino)-2-(quinazolin-4-ylamino)butanoic acid, or a hydrate thereof, comprising preparing a mixture of (S)-4-((2-methoxyethyl)(4-(5,6,7,8-tetrahydro-1,8-naphthyridin-2-yl)butyl)amino)-2-(quinazolin-4-ylamino)butanoic acid and tartaric acid in a solvent, thereby resulting in the crystal form of (S)-4-((2-methoxyethyl)(4-(5,6,7,8-tetrahydro-1,8-naphthyridin-2-yl)butyl)amino)-2-(quinazolin-4-ylamino)butanoic acid, or the hydrate thereof.

40. The method of claim 39, wherein the solvent comprises an alcohol.

41. The method of claim 40, wherein the alcohol comprises methanol.

42. The method of any one of claims 39-41, further comprising heating the mixture to about 50° C. for about 2 hours, and then cooling the mixture to about 25° C.

43. The method of claim 39, wherein the alcohol comprises isopropanol.

44. The method of claim 43, further comprising stirring the mixture at about 22-27° C. for about 2 days.

45. The method of any of claims 39-44, wherein the crystalline form is a cocrystal.

46. A pharmaceutical composition comprising the crystalline form of any one of claims 1-38, and a pharmaceutically acceptable carrier or excipient.

47. A kit comprising the crystalline form of any one of claims 1-38, or the pharmaceutical composition of claim 46, and instructions for the treatment of a fibrotic disease.

48. A method of treating a disease in an individual in need thereof comprising administering to the individual the crystalline form of any one of claims 1-38, or the pharmaceutical composition of claim 46, wherein the disease is selected from the group consisting of fibrosis, progressive pulmonary fibrosis, pulmonary fibrosis associated with rheumatoid arthritis, rheumatoid arthritis associated interstitial lung disease (RA-ILD), progressive familial intrahepatic cholestasis (PFIC), acute respiratory distress syndrome (ARDS), idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, fibrotic nonspecific interstitial pneumonia (NSIP), scleroderma, diabetic nephropathy, diabetic kidney disease, focal segmental glomerulosclerosis, chronic kidney disease, and Crohn's Disease.

49. The method of claim 48, wherein the disease comprises fibrosis.

50. The method of claim 49, wherein the fibrosis is pulmonary fibrosis, liver fibrosis, skin fibrosis, cardiac fibrosis, kidney fibrosis, gastrointestinal fibrosis, primary sclerosing cholangitis, or biliary fibrosis.

51. The method of claim 49, wherein the fibrosis is liver fibrosis, cardiac fibrosis, primary sclerosing cholangitis, or biliary fibrosis.

52. The method of claim 48 or 49, wherein the disease is progressive pulmonary fibrosis, pulmonary fibrosis associated with rheumatoid arthritis, idiopathic pulmonary fibrosis (IPF), radiation-induced pulmonary fibrosis, alcoholic liver disease induced fibrosis, or primary sclerosing cholangitis (PSC).

53. A method of treating a disease in an individual in need thereof comprising administering to the individual the crystalline form of any one of claims 1-38, or the pharmaceutical composition of claim 46, wherein the disease is liver fibrosis induced by pathogens, alcoholic steatosis induced liver fibrosis, cirrhosis, or biliary tract fibrosis.

54. A method of treating a disease in an individual in need thereof comprising administering to the individual the crystalline form of any one of claims 1-38, or the pharmaceutical composition of claim 46, wherein the disease is diabetic nephrosclerosis, hypertensive nephrosclerosis, or acute kidney injury from contrast induced nephropathy, glomerulonephritis, end-stage kidney disease, hearing loss, changes to the lens of the eye, hematuria, or proteinuria.

55. A method of treating a disease in an individual in need thereof comprising administering to the individual the crystalline form of any one of claims 1-38, or the pharmaceutical composition of claim 46, wherein the disease is scleroderma, systemic sclerosis, systemic and local sclerosis or scleroderma, keloids and hypertrophic scars, or post-surgical adhesions.

56. A method of treating a disease in an individual in need thereof comprising administering to the individual the crystalline form of any one of claims 1-38, or the pharmaceutical composition of claim 46, wherein the disease is atherosclerosis or restenosis, post myocardial infarction induced fibrosis, or inherited cardiomyopathy.

57. The method of any one of claims 48-52, wherein the disease is idiopathic pulmonary fibrosis.

58. The method of any one of claims 48-52, wherein the disease is progressive pulmonary fibrosis.

59. The method of any one of claims 48-52, wherein the disease is primary sclerosing cholangitis.

60. The method of claim 48, wherein the disease is interstitial lung disease.

61. The method of claim 48, wherein the disease is rheumatoid arthritis associated interstitial lung disease.

62. Use of the crystalline form of any one of claims 1-38 in the manufacture of a medicament for the treatment of a fibrotic disease.

63. Use of the crystalline form of any one of claims 1-38 in the manufacture of a medicament for the treatment of a disease mediated by at least one of αᵥβ₁ integrin and αᵥβ₆ integrin.

64. The crystalline form of any one of claims 1-38, or the pharmaceutical composition of claim 46, for use in treating a fibrotic disease.

65. The crystalline form of any one of claims 1-38, or the pharmaceutical composition of claim 46, for use in treating a disease mediated by at least one of αᵥβ₁ integrin and αᵥβ₆ integrin.
